(19) United States
(12) Patent Application Publication
(10) Pub. No.: US 2007/0269379 A1
(43) Pub. Date: Nov. 22, 2007

(54) Penetration Enhancer Combinations for Transdermal Delivery

(76) Inventors: Samir Mitragotri, Goleta, CA (US); Pankaj S. Karande, Somerville, MA (US); Amit K. Jain, Redwood City, CA (US)

Correspondence Address: Rober Berliner;Berliner & Associated, 555 W. Fifth Street, 31st Floor, Los Angeles, CA, 90013, US

(21) Appl. No.: 10/560,571

(22) PCT Filed: Jul. 21, 2004
(86) PCT No.: PCT/US04/23634

§ 371 Date: Feb. 2, 2007

Related U.S. Application Data
(60) Provisional application No. 60/560,717, filed on Jul. 23, 2003.

(51) Int. Cl. A61K 49/00; A61K 9/70

(52) U.S. Cl. 424/9.2; 424/449; 435/40.52; 436/149; 514/772; 514/777; 514/788; 514/789; 435/7.1

(57) ABSTRACT

A high throughput screening and isolation system identifies rare enhancer mixtures from a candidate pool of penetration enhancer combinations. The combinations are screened for high penetration but low irritation potential using a unique data mining method to find new potent and safe chemical penetration enhancer combinations. The members of a library of chemical penetration enhancer combinations are screened with a high throughput device to identify “hot spots”, particular combinations that show higher chemical penetration enhancement compared to neighboring compositions. The irritation potentials of the hot spot combinations are measured to identify combinations that also show low irritation potential. A active component, such as a drug, is then combined with the combination in a formulation which is tested for the ability of the drug to penetrate into or through skin. It is then assessed whether the formulation can deliver the quantity of drug required, and animal tests are conducted to confirm in vivo the ability of the chemical penetration enhancer combinations to facilitate transport of sufficient active molecules across the skin to achieve therapeutic levels of the active molecule in the animal's blood. The invention provides specific unique and rare mixtures of chemical penetration enhancers that enhance skin permeability to hydrophilic macromolecules by more than 50-fold without inducing skin irritation, such as combinations of sodium laurel ether sulfate and 1-phenyl piperazine, and combinations of N-lauryl sarcosine and Span 20/sorbitan monolaurate.

### CROSS REFERENCE TO RELATED PATENT APPLICATIONS

This application claims the benefit of Provisional Patent Application No. 60/560,717, filed Jul. 23, 2003.

### FIELD

The invention includes compositions for the delivery of active ingredients such as drugs into and through skin and other tissues and related screening methods.

### BACKGROUND

Skin is the largest organ of the human body and provides a painless and compliant interface for systemic drug administration. The transdermal route may provide advantages over injections and oral routes by increasing patient compliance and avoiding first pass metabolism, and may also provide sustained and controlled delivery over long times. However, after nearly four decades of extensive studies, the success of this technology remains stinted with only a limited number of transdermal products available in the market, all of which are based on low-molecular weight lipophilic drugs.

Development of transdermal products for macromolecules is primarily hindered by low skin permeability. Evolved to impede the flux of toxins into the body, skin naturally offers a very low permeability to the movement of foreign molecules across it. A unique hierarchical structure of lipid-rich matrix with embedded keratinocytes in the upper strata (15 μm) of skin, the stratum corneum (SC), is largely responsible for the barrier properties of skin. Several technological advances have been proposed in efforts to overcome this barrier. Examples include iontophoresis, sonophoresis, and use of chemical penetration enhancers (CPE). CPEs can provide advantages including design flexibility with formulation chemistry, possibility of patch application over a large area (>10 cm²) and ability to work without external physical delivery mechanisms. Several different classes of CPEs including surfactants, fatty acids and fatty esters have been studied in the literature and more than 250 chemicals have been identified as enhancers that can increase skin permeability. However, only a few induce a significant (therapeutic) enhancement of drug transport. Moreover, the permeability of skin to foreign molecules shows a trend to decrease rapidly with the molecular weight (MW) of the foreign molecule. Transdermal delivery of high molecular weight drugs is therefore especially difficult and all current drugs delivered with patch technologies have a molecular weight of less than 500 Daltons. Bos et al. (2000). The problem of development of transdermal approaches for drug delivery is further aggravated by the fact that potent enhancers are usually also potent irritants to skin and are thus physiologically incompatible.

Pushing the envelope on enhancement efficiencies with single enhancers inevitably leads to a compromise on safety issues. Potent CPEs usually enhance skin permeability by disrupting the SC lipid bilayers. Since the SC is comprised of non-viable, keratinized cells, disruption of its lipid bilayers is itself not sufficient to induce irritation. However, CPEs are usually not selective towards SC lipids and eventually disrupt viable epidermal cells thereby inducing irritation due to the interstitial release of cytokines and by triggering other inflammatory responses. Attempts have been made to engineer physico-chemical properties of CPE molecules to enhance potency without affecting irritancy, but without much success.

A number of approaches for improving the penetration of drugs using liposomes and related systems have been pursued over the years and have recently been reviewed by Hadgraft and further details may be found in the book of Williams, especially Chapter 5. Hadgraft (2003); Williams (2003). Mezei and Gulasekharam in 1980 performed early work to show the potential value of liposomally encapsulated drugs for topical therapy. Mezei et al. (1980). U.S. Pat. Nos. 5,540,943 and 5,716,638 are said to describe ethosomes which are characterized as “soft” vesicles formed from phospholipids in the presence of water and alcohol and sometimes glycols and contain claims directed towards liposomal compositions for medical or cosmetic use. U.S. Pat. No. 6,165,500 is said to describe the use of elastic, deformable mini-droplets called Transfersomes for transporting medical agents through the skin of a mammal. U.S. Pat. Nos. 5,853,755 and 5,993,851 are said to describe biphasic multilamellar lipid vesicles and contain claims directed towards liposomal compositions for topical administration of compounds and methods for preparing liposomes having a central core compartment containing an oil in water emulsion. However, liposomal formulations have yet to appear in FDA approved patch products for transdermal delivery of drugs, in spite of the fact that this area has been under development for more than 20 years. Williams (2003).

Overcoming the SC barrier safely and reversibly is a fundamental problem that persists in the field of transdermal delivery. In the absence of fundamental knowledge of these interactions, rapid methods to screen various enhancers are of value. Most drugs bind strongly and selectively to a target protein. Recent advances in biotechnology have allowed rapid screening of thousands of drugs for their ability to bind to such protein targets. Ng, et al. (1999); Verdine, et al. (1996). Through the development of combinatorial drug discovery, new drugs, including low-molecular weight analogs of proteins and peptides, are being continually developed. Zhang, et al., (1999). However, the ability to deliver these drugs is still evaluated by traditional experiments. In these experiments, the biological membrane under consideration, such as the skin for transdermal drug delivery or the intestine for oral drug delivery, is placed in a diffusion cell and transport across this membrane is measured over several hours or days. Bronaugh, et al., (1985). In many cases, additional experiments are performed to try to assess the effect of the formulation on membrane permeability. During this process, various formulations are utilized in an effort to optimize drug bioavailability. The objective of this optimization is the identification of a formulation that can deliver the required therapeutic dose into the body. This process is based on traditional experiments and is time-consuming as well as expensive. Availability of a rapid screening method to determine trans-membrane transport of drugs would greatly facilitate the development of drug delivery systems. An ability to discover formulations that can deliver a much wider range of drugs transdermally with low irritation would be very important, enabling the attractive benefits of transdermal delivery to be realized for a much wider range of therapeutics.

Transdermal patches are often used in the delivery of drugs through the skin. Patches can be categorized into several types depending on how the drug is incorporated into the device and include: (i) those in which the drug is in an adhesive; (ii) those in which the drug is in a matrix; and (iii) those in which the drug is in a reservoir. Williams (2003). The formulations utilized in patch and topically applied medications contain multiple components and a typical drug formulation may contain anywhere from 3-15 components, including the drug. In order to optimize the concentration of these components of a formulation containing, for example six components, an experimental design is required that may include, for example, five levels of concentration of each component. In order to determine the optimal concentration of these components, 5⁶ experiments are required; that is, about 15,000 experiments. Note that in a typical formulation development project, testing a system containing more than six components is not unusual. Thus, the number of experiments required for optimization may be extremely large. Although reducing the parameter space by either eliminating some of the components or by reducing the levels of each component in the experimental design may lessen the number of experiments needed to be done, it greatly increases the likelihood of missing other potentially important formulations. A typical transdermal transport experiment lasts for at least 24 hours and uses about a 2 cm² piece of skin. It is customary to run about 15-20 transport experiments at a time. At this rate, it would take hundreds of days to screen all 15,000 combinations.

Most molecules known as potent chemical penetration enhancers in the literature are also potent irritants. Very few molecules that show therapeutically significant enhancements enhancement of penetration are physiologically compatible. This is a limiting step in exploiting transdermal delivery as an efficient delivery mode. Combinations of two or more penetration enhancers may also be used, and may be more effective in increasing transdermal transport compared to each of them alone. Mollgaard (1993). Several amphiphilic molecules enhance skin permeability via temporary disruption of the lipid structure of the stratum corneum. However, they have found limited clinical acceptance as they almost invariably induce skin irritation, given that the plasma membranes of live cells in the epidermis have similar compositions to the lipid layers in the stratum corneum.

There is a need for new penetration enhancer compositions that can extend the range of drugs that are suitable for delivery in topical and transdermal formats as well as effective methods to identify mixtures of penetration enhancers that significantly enhance skin permeability for a much broader range of active ingredients without inducing skin irritation.

### SUMMARY

The inventions described and claimed herein have many attributes and embodiments including, but not limited to, those set forth or described or referenced in this Summary. The inventions described and claimed herein are not limited to or by the features or embodiments identified in this Summary, which is included for purposes of illustration only and not restriction.

The present invention employs, for example, a system known as “in vitro skin impedance guided high throughput” (INSIGHT) to perform high throughput experimentation (HTE), allowing the identification of rare penetration enhancer mixtures from a colossal candidate pool. INSIGHT uses high throughput skin impedance measurements (as described in Karande et al, (2002), and in International Publication Number WO 02/16941 A2). A library of CPE formulations are created by dissolving or dispersing CPEs in varying concentrations in one or more vehicles. The CPE formulations may be screened for high penetration enhancement but low irritation potential using a unique data mining method to identify or confirm new potent and safe penetration enhancers.

In one embodiment of the invention, hot spots may be identified, which are places in compositional space (defined by varying the concentrations of a set of CPEs) within which pronounced penetration enhancement is found as a consequence of a combination of constituent CPEs in the CPE formulation.

Hot spots are regions in composition space of a CPE formulation where enhanced permeability of a membrane is observed as the concentrations of at least two chemical penetration enhancers within the CPE formulation are varied. Hot spots are associated with relatively sharp permeability maxima and by relatively large values compared with the permeability produced by each of the individual CPEs in isolation. Mathematically, a so-called synergy value, S, may be calculated for a composition containing at least two CPEs, A and B, according to the following equation

$$S = \frac{{ER}_{A + B} ( X , Y )}{X \cdot {ER}_{A} ( Y ) + ( 1 - X ) \cdot {ER}_{B} ( Y )} ,$$

where ER_{A+B}(X,Y) is the enhancement ratio obtained with the formulation containing CPEs A and B, Y stands for the combined total concentration of A and B measured in wt/vol, X stands for the weight fraction of A calculated according to the amount of A in formulation (expressed in wt/vol) divided by Y and ER_{A}(Y) and ER_{B}(Y) are the enhancement ratios obtained when the CPEs A and B are replaced in the formulation with pure components A and B, respectively at concentration Y. Enhancement ratios and therefore synergy values vary as a function of the time that a formulation has been in contact with a membrane and it is often convenient to evaluate 24-hour synergy values computed by consideration of enhancement ratios observed after a formulation has been in contact with skin for 24 hours. Permeability maxima in the two-dimensional concentration space of two penetration enhancers A and B with 24 hour S values of 2 or more, or more preferably 4 or more, can be used to identify hot spots. It is to be understood that in a system with N permeation enhancers there are N(N−1)/2 ways of forming binary A-B pairs of permeation enhancers. S values may be calculated by the above equation for each pair of CPEs in the formulations and hot spots may be identified by varying the concentrations of different pairs of CPEs in the formulation.

Many hot spots represent rare mixtures of CPEs that exhibit potent ability to increase the permeability of the stratum corneum. It has been discovered that some hot spots also have low irritation potential. Without being bound by theory, a possible explanation of this phenomenon is that the hot spot formulations evolve into a relatively non-disruptive formulation in the epidermis, perhaps due to differential retention of the components in the CPE mixture in the stratum corneum versus the epidermis. The formulations that comprise a combination of CPEs associated with a hot spot and which induce no more than modest skin irritation are referred to as “synergistic” combinations of penetration enhancers (SCOPE). Synergism may be seen but is not a requirement of the invention. SCOPE formulations are conceptually distinct from empirically formulated enhancer combinations reported in the literature, which rarely have enhanced penetration without inducing irritation.

Another embodiment of the present invention provides a methodology for identifying and for discovering hot spots and SCOPE formulations.

A further embodiment of the present invention provides a methodology for discovering SCOPE formulations suitable for inter- or trans-dermal delivery of drugs.

The method of the present invention provides a procedure comprising, for example, all or some of the following steps:
(a) Obtaining a large diverse library of CPE combinations, which can be constructed, for example, by random selection of CPEs from known or other CPEs, selecting one or more vehicles and combining the selected CPEs and vehicle in different ratios to make the diverse library;
(b) Screening the elements of the library with a HTE device for their ability to increase skin penetration;
(c) Analyzing the skin penetration data for hot spots to select CPE combinations for further analysis;
(d) Measuring the irritation potential of the hot spot CPE combinations. This can be done by any known method. For example, hot spot CPE combinations can be placed, 24 at a time, on a culture of normal human derived epidermal keratinocytes and the viability of the cells measured at the end of the study period, e.g., 4 to 24 hours, using a MatTek device (MatTek Corporation, 200 Homer Avenue, Ashland, Mass. 01721, www.mattek.com).
(e) Identifying from (d) those formulations that are SCOPE formulations, that is, that show low irritation potential.
(f) Combining one or more identified formulations with a selected drug and testing for penetration through skin. This can be done by any known method. For example, the drug-formulation combination can be placed on porcine or human skin and penetration of the drug through the skin can be measured after a period of 24 to 96 hours using Franz diffusion cells.
(g) Determining whether the formulation can deliver the necessary drug amount, e.g., by comparison with published data.
(h) Conducting animal testing to confirm the ability of the enhancer combinations to deliver sufficient drug molecules across the skin to achieve therapeutic levels of the drug in the animal's blood. For example, in vivo experiments in hairless rats can be performed using leuprolide acetate as a model drug.

The concept of selective testing of hot spots for low irritation potential is a powerful tool for identifying combinations of penetration enhancers that have the ability to rapidly penetrate the SC but which have low irritation potential. In prior screening methods, where single formulations were tested, results were indicative only of the ability of the formulation to penetrate the SC. It has surprisingly been found that hot spots identified from the evaluation of formulations containing CPEs not only identify strong SC penetrators but sometimes also SC penetrator combinations with low irritation potential.

The invention also provides combinations that can be mixed with a selected drug or other active component to greatly facilitate its transport through the and into or through the epidermis.

The methods of the present invention apply generally to the inter- or trans-dermal delivery of compounds. Thus, by way of example but not limitation, the present invention applies to the inter- and transdermal delivery of small molecule, lipophilic drugs, but of lipophilic drugs of a broad range of molecular weights, up to several thousand Daltons and beyond, of non-lipophilic or hydrophilic drugs, also of a broad range of molecular weights, of molecular or other ingredients for cosmetic application, of diagnostic agents, of genetic material such as DNA, of nanoparticulate materials, and the like. The present invention relates to compounds to be delivered for the benefit of skin tissues, for example, such as for dermatological, antibiotic, antifungal or cosmetic application, as well as to compounds to be delivered, for example, for systemic application. The present invention is of particular benefit for routes for the delivery of compounds into and through skin, and the present invention is also of benefit for routes for the delivery of compounds into or through tissues or other types, such as mucosal tissue, as well as of synthetic membranes. As a consequence the present invention also provides methods for treating diseases. Further embodiments of the present invention are transdermal patches containing formulations with potent ability to permeabilize skin and low irritation potential that may be used to deliver drugs or other active components.

Another embodiment of the invention provides specific SCOPE formulations, certain mixtures of penetration enhancers that enhance skin permeability to hydrophilic macromolecules (MW˜1 kDa-5 kDa) by more than 50-fold without inducing skin irritation. These include combinations of sodium laurel ether sulfate and 1-phenyl piperazine, and combinations of N-lauryl sarcosine and Span 20/sorbitan monolaurate.

### BRIEF DESCRIPTION OF THE DRAWINGS

FIG. 1 is a flow chart showing a sequence of steps useful for the identification of low irritation penetration enhancers according to a preferred embodiment of the present invention;

FIG. 2 is a schematic of a device that may be used for high throughput screening of formulations.

FIG. 3 is a table of chemical penetration enhancers that have been used to construct one library of penetration enhancer combinations;

FIG. 4 is a table that classifies the chemical penetration enhancers listed in the table in FIG. 3 into 8 separate categories and shows how these chemical penetration enhancers were divided into four blocks to assist in the construction of a library;

FIG. 5 is a histogram showing the frequency with which enhancement ratios in different ranges were observed in a data set containing over 20,000 conductivity enhancement ratios;

FIG. 6 shows potency phase maps for six pairs of chemical penetration enhancers;

FIG. 7 is a table showing maximum conductivity enhancement ratios and maximum synergy ratios for 11 different combinations of chemical penetration enhancers;

FIG. 8 shows a graph of irritation potential versus conductivity enhancement ratio for a series of individual chemical penetration enhancers and a series of chemical penetration enhancer combinations;

FIG. 9 is a table showing a mapping between the numeral symbols used to label data points in FIG. 8 and the chemical penetration enhancer combinations;

FIG. 10 is a table showing a mapping between the letter symbols used to label data points in FIG. 8 and the individual chemical penetration enhancers;

FIG. 11 shows a potency phase map of the chemical penetration enhancer pair SLA PP;

FIG. 12 shows a potency phase map of the chemical penetration enhancer pair NLS S20;

FIG. 13 shows a permeability enhancement ratio for inulin measured with a Franz diffusion cell for the chemical penetration enhancer pair NLS S20 and tape stripped skin;

FIG. 14 shows the permeability enhancement ratio of inulin measured with a Franz diffusion cell for formulations containing SLA PP, NLS S20 and tape stripped skin. In contrast to the data presented in FIG. 13, these data have not been corrected for the amount of inulin that was trapped in the skin;

FIG. 15 shows irritation potential versus conductivity enhancement ratio for two SCOPE formulations based on SLA PP and NLS S20 compared to that of formulations containing the individual chemical penetration enhancers SLA, PP, NLS and S20;

FIG. 16 shows Franz diffusion cell data on the dependence on molecular weight of skin permeability for a range of molecules. Open circles show permeability of untreated skin reported in the literature to a variety of hydrophilic solutes. Open squares show skin permeability achieved for a range of test molecules utilizing formulations containing SLA and PP. Closed circles show skin permeability for the same test molecules in the SLA PP formulation that have been corrected to account for the amount of test molecule that was trapped in the skin;

FIG. 17 shows the plasma concentration of leuprolide as a function of time after placement of leuprolide patches containing SLA:PP and hyaluronic acid (closed symbols) and control patches containing leuprolide and hyaluronic acid (open symbols) on the skin of hairless rats;

FIG. 18 shows micrographs of hairless rat skin after application of patches containing a control formulation based on PBS (FIG. 18 (A)), a SCOPE containing SLA and PP (FIG. 18 (B)), and a formulation containing SLS (FIG. 18 (C)); and

FIG. 19 shows Franz diffusion cell measurement of the permeability of corticosterone across porcine skin utilizing a formulation based on NLS S20 compared to that achieved utilizing a formulation based on PBS.

### DETAILED DESCRIPTION

The following terms have the following meanings when used herein and in the appended claims. Terms not specifically defined herein have their art recognized meaning.

“Active component” means a substance or compound that imparts a primary utility to a composition or formulation when the composition or formulation is used for its intended purpose. Examples of active components include pharmaceuticals, vitamins, ultra violet (“UV”) radiation absorbers, cosmeceuticals, alternative medicines, skin care actives, and nutraceuticals. Active components can, by way of example but not limitation, be small molecules, proteins or peptides, genetic material, such as DNA or RNA, diagnostic or sensory compounds, agrochemicals, the active component of a consumer product formulation, or the active component of an industrial product formulation.

“Active component formulation” means a formulation which contains one or more active components.

“Array” or “sample array” means a plurality of samples associated under a common experiment, or the physical arrangement of a plurality of vessels used to contain samples in a given experiment.

“Automated” or “automatically” refers to the use of non-human means such as computer software and robotics to achieve one or more operations such as adding, mixing, dispensing or analyzing the samples, components, and specimens or diffusion products.

“Body surface” refers to skin or mucosal tissue.

“Carriers” or equivalently “vehicles” as used herein refer to carrier materials suitable for topical or transdermal drug administration or for formulating samples for use in high throughput experimentation. Carriers and vehicles useful herein include any such material known in the art that is generally nontoxic and does not interact with other components of the composition in a deleterious or unwanted manner. Vehicles may contain one or more excipients and may also contain one or more chemical penetration enhancers. Carriers and vehicles can be, for example, semi-solids, liquids, solvents, solutions, gels, foams, pastes, ointments, triturates, suspensions, or emulsions.

“Component” means any substance or compound. A component can be active or inactive.

“Enhancement ratio” or equivalently “skin conductivity enhancement ratio” means the ratio σₜ/σ₀ where σ₀ is an initial skin conductivity observed after a formulation has been brought into contact with skin and σₜ is skin conductivity observed after an incubation time t. The enhancement ratio is a function of time and the term “t-hour enhancement ratio” is understood to mean an enhancement ratio measured after an incubation time of t hours, where t hours may be any period of time over which enhancement ratios may be reasonably measured. As explained below enhancement ratios can be conveniently measured using high throughput devices or Franz diffusion cells by measuring ratios in currents that flow across a skin sample in response to an applied voltage. It is understood that it may be necessary to repeat skin conductivity measurements on a number of separate skin samples to obtain a statistically meaningful result, due to experimental errors that may be introduced in the measurement process, for example, as a consequence of variability in skin samples used in the experiment.

“Excipient” refers to inactive substances used to formulate pharmaceuticals as a result of processing or manufacture or used by those of skill in the art to formulate pharmaceuticals, alternative medicines, cosmeceuticals, cosmetics, personal care products, dietary supplements, and nutraceuticals for administration to animals or humans.

Preferably, excipients are approved for or considered to be safe for human and animal administration. Examples of suitable excipients include, but are not limited to, acidulents, such as lactic acid, hydrochloric acid, and tartaric acid; solubilizing components, such as non-ionic cationic, and anionic surfactants; absorbents, such as bentonite, cellulose, and kaolin; alkalizing components, such as diethanolamine, potassium citrate, and sodium bicarbonate; anticaking components, such as calcium phosphate tribasic, magnesium trisilicate, and talc; antimicrobial components, such as benzoic acid, sorbic acid, benzyl alcohol, benzethonium chloride, bronopol, alkyl parabens, cetrimide, phenol, phenylmercuric acetate, thimerosol, and phenoxyethanol; antioxidants, such as ascorbic acid, alpha tocopherol, propyl gallate, and sodium metabisulfite; binders, such as acacia, alginic acid, carboxymethyl cellulose, hydroxyethyl cellulose; dextrin, gelatin, guar gum, magnesium aluminum silicate, maltodextrin, povidone, starch, vegetable oil, and zein; buffering components, such as sodium phosphate, malic acid, and potassium citrate; chelating components, such as EDTA, malic acid, and maltol; coating components, such as adjunct sugar, cetyl alcohol, poly-vinyl alcohol, carnauba wax, lactose maltitol, titanium dioxide; controlled release vehicles, such as microcrystalline wax, white wax, and yellow wax; desiccants, such as calcium sulfate; detergents, such as sodium lauryl sulfate; diluents, such as calcium phosphate, sorbitol, starch, talc, lactitol, polymethacrylates, sodium chloride, and glyceryl palmitostearate; disintegrants, such as colloidal silicon dioxide, croscarmellose sodium, magnesium aluminum silicate, potassium polacrilin, and sodium starch glycolate; dispersing components, such as poloxamer 386, and polyoxyethylene fatty esters (polysorbates); emollients, such as cetearyl alcohol, lanolin, mineral oil, petrolatum, cholesterol, isopropyl myristate, and lecithin; emulsifying components, such as anionic emulsifying wax, monoethanolamine, and medium chain triglycerides; flavoring components, such as ethyl maltol, ethyl vanillin, fumaric acid, malic acid, maltol, and menthol; humectants, such as glycerin, propylene glycol, sorbitol, and triacetin; lubricants, such as calcium stearate, canola oil, glyceryl palmitostearate, magnesium oxide, poloxymer, sodium benzoate, stearic acid, and zinc stearate; solvents, such as alcohols, benzyl phenylformate, vegetable oils, diethyl phthalate, ethyl oleate, glycerol, glycofurol, polyethylene glycol, tartazine, triacetin; stabilizing components, such as cyclodextrins, albumin, xanthan gum; and tonicity components, such as glycerol, dextrose, potassium chloride, and sodium chloride; and mixtures thereof. Excipients include those that alter the rate of absorption, bioavailability, or other pharmacokinetic properties of pharmaceuticals, dietary supplements, alternative medicines, or nutraceuticals. Other examples of suitable excipients, such as binders and fillers are listed in Remington's Pharmaceutical Sciences, 18th Edition, Ed. Alfonso Gennaro, Mack Publishing Co. Easton, Pa., 1995 and Handbook of Pharmaceutical Excipients, 3rd Edition, Ed. Arthur H. Kibbe, American Pharmaceutical Association, Washington D.C. 2000, both of which are incorporated herein by reference. Excipients that are typically used in the formation of transdermal delivery devices, and therefore particularly useful for formulation of the samples of the present invention, are penetration enhancers, adhesives and solvents

“High throughput” refers to the number of samples generated or screened in a given time period as described herein, typically at least 10, more typically at least 50 to 100, and preferably more than 1000 samples. The high throughput methods of the present invention can be performed using various means and various forms of samples. Typically, the methods are performed either with liquid samples or with solid or semi-solid samples.

“Irritation antergy factor” between two CPEs in a formulation is calculated according to

$$A = \frac{X \cdot {IP}_{A} ( Y ) + ( 1 - X ) \cdot {IP}_{B} ( Y )}{{IP}_{A + B} ( X , Y )} ,$$

where IP_{A+B}(X,Y) is the irritation potential measured for the formulation containing CPEs A and B, Y stands for the amount of the combination of A and B expressed in wt/vol and X stands for the amount of A in formulation (expressed in wt/vol) divided by Y. IP_{A}(Y) and IP_{B}(Y) are measured by preparing formulations whose composition is the same as that containing the CPEs A and B except that CPEs A and B are replaced with either pure component A at a wt/vol of Y or pure component B at a wt/vol of Y. IP_{A}(Y) and IP_{B}(Y) are then the irritation potentials measured for the formulation in which A, but not B, is present and B, but not A, is present, respectively. Irritation potential can be measured according to a number of different methods and the calculated irritation antergy factor of a formulation will depend on which method for measuring irritation potential is employed. Preferably, irritation antergy factor is calculated using irritation potentials measured as an MTT 4-hour cell viability percentage.

“Irritation potential” means a numerical measure of irritation of a formulation, which tends to increase in value as the degree of irritancy of the formulation increases. Irritation potential may be measured in vivo using animals or humans. For example, in vivo irritation potential in humans may be measured by the 21-day cumulative irritation test. Berger (1982). Irritation potential may also be measured in vitro utilizing the methods discussed in more detail below. In one approach to measurement of irritation potential, reconstructed human epidermis equivalents may be employed such as EpiDerm™ or EPISKIN™. Faller (2002).

“MTT 4-hour cell viability percentage” means irritation potential of a formulation as measured as a percentage of cell viability after 4 hours of contact with the formulation on the EpiDerm™ skin model (Mattek Corporation, Ashland, Mass. www.mattek.com) assayed using a methyl thiazol tetrazolium (MTT) uptake assay according to the protocol provided in the paper of Faller et al. Faller (2002). MTT 4-hour cell viability percentages are generally expected to fall in the range of 0-100%.

“Mucosa” means a mucous membrane that covers the inside of a hollow organ such as the membranes covering the oral cavity, the nasal cavity, the rectum and the vagina.

“Library” means a plurality of samples.

“Permeation enhancer” or, equivalently, “penetration enhancer,” “chemical penetration enhancer” or “CPE” means a substance used to modify, usually to increase, the rate of permeation through skin or other tissue of one or more products in a formulation, and includes all such substances now known or later developed or discovered. See Santus et al. (1993) and Williams (2003). Various enhancers are listed below. These enhancers are compiled from over 350 references and have been classified into several categories and subcategories based on their structure or their effect on permeability:

Surfactants: These are amphiphilic molecules with a hydrophilic head and a hydrophobic tail group. The tail length and the chemistry of the head group play an important role in determining their effect on skin permeability. Surfactants can be categorized into four groups, cationic, anionic, non-ionic, and zwitterionic depending on the charge on the head group. Prominent examples of surfactants that have been used for transdermal delivery include: Brij (various chain lengths), HCO-60 surfactant, Hydroxypolyethoxydodecane, Lauryl sarcosine, Nonionic surface active agents, Nonoxynol, Octoxynol, Phenylsulfonate, Pluronic, Polyoleates (nonionic surfactants), Rewopal HV10, Sodium laurate, Sodium oleate, Sorbitan dilaurate, Sorbitan dioleate, Sorbitan monolaurate, Sorbitan monooleates, Sorbitan trilaurate, Sorbitan trioleate, Span 20, Span 40, Span 85, Synperonic NP, Triton X-100, Tweens, Sodium alkyl sulfates, and alkyl ammonium halides.

Azone and related compounds: These compounds are also amphiphilic and possess a nitrogen molecule in their head group (preferably in the ring). The presence of a nitrogen atom in a ring creates a bulky polar head group with the potential for strong disruption of stratum corneum. Examples of such compounds include N-Acyl-hexahydro-2-oxo-H-azepines, N-Alkyl-dihydro-1,4-oxazepine-5,7-diones, N-Alkylmorpholine-2,3-diones, N-Alkylmorpholine-3,5-diones, Azacycloalkane derivatives (-ketone, -thione), Azacycloalkenone derivatives, 1-[2-(Decylthio)ethyl]azacyclopentan-2-one (HPE-101), N-(2,2), Dihydroxyethyl dodecylamine, 1-Dodecanoylhexahydro-1-H-azepine, 1-Dodecyl azacycloheptan-2-one (azone or laurocapram), N-Dodecyl diethanolamine, N-Dodecyl-hexahydro-2-thio-1H-azepine, N-Dodecyl-N-(2-methoxyethyl)acetamide, N-Dodecyl-N-(2-methoxyethyl) isobutyramide, N-Dodecyl-piperidine-2-thione, N-Dodecyl-2-piperidinone, N-Dodecyl pyrrolidine-3,5-dione, N-Dodecyl pyrrolidine-2-thione, N-Dodecyl-2-pyrrolidone, 1-Farnesylazacycloheptan-2-one, 1-Farnesylazacyclopentan-2-one, 1-Geranyl azacycloheptan-2-one, 1, Geranylazacyclopentan-2-one, Hexahydro-2-oxo-azepine-1-acetic acid esters, N-(2, Hydroxyethyl)-2-pyrrolidone, 1-Laurylazacycloheptane, 2-(1-Nonyl)-1,3-dioxolane, 1-N-Octylazacyclopentan-2-one, N-(1-Oxododecyl)-hexahydro-1H-azepine, N-(1, Oxododecyl)-morpholines, 1-Oxohydrocarbyl-substituted azacyclohexanes, N-(1-Oxotetradecyl)-hexahydro-2-oxo-1H-azepine, and N-(1 Thiododecyl)-morpholines.

Solvents and related compounds: These molecules are solubility enhancers. Some of them also extract lipids, thereby increasing skin permeability. Examples of solvents include Acetamide and derivatives, Acetone, n-Alkanes (chain length between 7 and 16), Alkanols, diols, short-chain fatty acids, Cyclohexyl-1,1-dimethylethanol, Dimethyl acetamide, Dimethyl formamide, Ethanol, Ethanol/D-limonene combination, 2-Ethyl-1,3-hexanediol, Ethoxydiglycol (transcutol), Glycerol, Glycols, Lauryl chloride, Limonene, N-Methylformamide, 2-Phenylethanol, 3-Phenyl-1-propanol, 3-Phenyl-2-propen-1-ol, Polyethylene glycol, Polyoxyethylene sorbitan monoesters, Polypropylene glycol 425, Primary alcohols (tridecanol), Procter & Gamble system: small polar solvents (1,2-propane diol, butanediol, C3-6 triols or their mixtures and a polar lipid compound selected from C16 or C18 monounsaturated alcohol, C16 or C18 branched saturated alcohol and their mixtures), Span 20, Squalene, Triacetin, Trichloroethanol, Trifluoroethanol, Trimethylene glycol, Xylene, DMSO and related compounds.

Fatty alcohols, fatty acids, fatty esters, and related structures: These molecules are classic bilayer fluidizers. Examples of these enhancers include Aliphatic alcohols, Decanol, Lauryl alcohol (dodecanol), Linolenyl alcohol, Nerolidol, 1-Nonanol, n-Octanol, Oleyl alcohol, Butyl acetate, Cetyl lactate, Decyl N,N-dimethylamino acetate, Decyl N,N-dimethylamino isopropionate, Diethyleneglycol oleate, Diethyl sebacate, Diethyl succinate, Diisopropyl sebacate, Dodecyl N,N-dimethylamino acetate, Dodecyl (N,N-dimethylamino)-butyrate, Dodecyl N,N-dimethylamino isopropionate, Dodecyl 2-(dimethylamino)propionate, EO-5-oleyl ester, Ethyl acetate, Ethylaceto acetate, Ethyl propionate, Glycerol monoethers, Glycerol monolaurate, Glycerol monooleate, Glycerol monolinoleate, Isopropyl isostearate, Isopropyl linoleate, Isopropyl myristate, Isopropyl myristate/fatty acid monoglyceride combination, Isopropyl myristate/ethanol/L-lactic acid (87:10:3) combination, Isopropyl palmitate, Methyl acetate, Methyl caprate, Methyl laurate, Methyl propionate, Methyl valerate, 1-Monocaproyl glycerol, Monoglycerides (medium chain length), Nicotinic esters (benzyl), Octyl acetate, Octyl N,N-dimethylamino acetate, Oleyl oleate, n-Pentyl N-acetylprolinate, Propylene glycol monolaurate, Sorbitan dilaurate, Sorbitan dioleate, Sorbitan monolaurate, Sorbitan monooleates, Sorbitan trilaurate, Sorbitan trioleate, Sucrose coconut fatty ester mixtures, Sucrose monolaurate, Sucrose monooleate, Tetradecyl N,N-dimethylamino acetate, Alkanoic acids, Capric acid, Diacid, Ethyloctadecanoic acid, Hexanoic acid, Lactic acid, Lauric acid, Linoelaidic acid, Linoleic acid, Linolenic acid, Neodecanoic acid, Oleic acid, Palmitic acid, Pelargonic acid, Propionic acid, Vaccenic acid, α-Monoglyceryl ether, EO-2-oleyl ether, EO-5-oleyl ether, EO-10-oleyl ether, Ether derivatives of polyglycerols and alcohols (1-O-dodecyl-3-O-methyl-2-0-(29, 39-dihydroxypropyl)glycerol), L-α-amino-acids, Lecithin, Phospholipids, Saponin/phospholipids, Sodium deoxycholate, Sodium taurocholate, and Sodium tauroglycocholate.

Others: Aliphatic thiols, Alkyl N,N-dialkyl-substituted amino acetates, Anise oil, Anticholinergic agent pretreatment, Ascaridole, Biphasic group derivatives, Bisabolol, Cardamom oil, 1-Carvone, Chenopodium (70% ascaridole), Chenopodium oil, 1,8 Cineole (eucalyptol), Cod liver oil (fatty acid extract), 4-Decyloxazolidin-2-one, Dicyclohexylmethylamine oxide, Diethyl hexadecylphosphonate, Diethyl hexadecylphosphoramidate, N,N-Dimethyl dodecylamine-N-oxide, 4,4-Dimethyl-2-undecyl-2-oxazoline, N-Dodecanoyl-L-amino acid methyl esters, 1,3-Dioxacycloalkanes, (SEPAs), Dithiothreitol, Eucalyptol (cineole), Eucalyptus oil, Eugenol, Herbal extracts, Lactam N-acetic acid esters, N-Hydroxyethalaceamide, 2-Hydroxy-3-oleoyloxy-1-pyroglutamyloxypropane, Menthol, Menthone, Morpholine derivatives, N-Oxide, Nerolidol, Octyl-β-D-(thio)glucopyranosides, Oxazolidinones, piperazine derivatives, Polar lipids, Polydimethylsiloxanes, Poly [2-(methylsulfinyl)ethyl acrylate], Polyrotaxanes, Polyvinylbenzyldimethylalkylammonium chloride, Poly(N-vinyl-N-methyl acetamide), Prodrugs, Saline, Sodium pyroglutaminate, Terpenes and azacyclo ring compounds, Vitamin E (α-tocopherol), Ylang-ylang oil, N-Cyclohexyl-2-pyrrolidone, 1-Butyl-3-dodecyl-2-pyrrolidone, 1,3-Dimethyl-2-imidazolikinone, 1,5 Dimethyl-2-pyrrolidone, 4,4-Dimethyl-2-undecyl-2-oxazoline, 1-Ethyl-2-pyrrolidone, 1-Hexyl-4-methyloxycarbonyl-2-pyrrolidone, 1-Hexyl-2-pyrrolidone, 1-(2 Hydroxyethyl)pyrrolidinone, 3-Hydroxy-N-methyl-2-pyrrolidinone, 1-Isopropyl-2-undecyl-2-imidazoline, 1-Lauryl-4-methyloxycarbonyl-2-pyrrolidone, N-Methyl-2-pyrrolidone, Poly(N-vinylpyrrolidone), Pyroglutamic acid esters, Acid phosphatase, Calonase, Orgelase, Papain, Phospholipase A-2, Phospholipase C and Triacylglycerol hydrolase.

“Penetration enhancement” means a measure of the degree to which a formulation is successful in increasing the permeability of skin, mucosa or a test membrane.

“Pharmaceutical” or, used interchangeably, “drug” means any substance or compound that has a therapeutic, disease preventive, diagnostic, or prophylactic effect when administered to an animal or a human. The term pharmaceutical includes prescription drugs and over the counter drugs. The molecular structures of drugs can often be characterized as small molecules, peptides, proteins and antibodies although other structures also include, for example, oligonucleotides and polysaccharides. Pharmaceuticals suitable for use in the invention include those now known or later developed. Examples of pharmaceuticals for use with SCOPE formulations include, but are not limited to, drugs of the following types: adrenergic agent; adrenocortical steroid; adrenocortical suppressant; aldosterone antagonist; amino acid; anabolic; analeptic; analgesic; anesthetic; anorectic; anti-acne agent; anti-adrenergic; anti-allergic; anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; antidiarrheal; antidiuretic; anti-emetic; anti-epileptic; antifibrinolytic; antifungal; antihemorrhagic; antihistamine; antihyperlipidemic; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; antiulcerative; antiviral; appetite suppressant; blood glucose regulator; bone resorption inhibitor; bronchodilator; cardiovascular agent; cholinergic; depressant; diagnostic aid; diuretic; dopaminergic agent; estrogen receptor agonist; fibrinolytic; fluorescent agent; free oxygen radical scavenger; gastric acid suppressant; gastrointestinal motility effector; glucocorticoid; hair growth stimulant; hemostatic; histamine H2 receptor antagonists; hormone; hypocholesterolemic; hypoglycemic, hypolipidemic; hypotensive; imaging agent; immunizing agent; immunomodulator; immunoregulator; immunostimulant; immunosuppressant, keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; psychotropic; radioactive agent; scabicide; sclerosing agent; sedative; sedative-hypnotic; selective adenosine Al antagonist; serotonin antagonist; serotonin inhibitor; serotonin receptor antagonist; steroid; thyroid hormone; thyroid inhibitor; thyromimetic, tranquilizer; amyotrophic lateral sclerosis agent; cerebral ischemia agent; Paget's disease agent; unstable angina agent; vasoconstrictor; vasodilator; wound healing agent, xanthine oxidase inhibitor.

Specific examples of pharmaceuticals that may be included within formulations of the invention, both alone or in combination, include but are not limited to:

Adrenergic: Adrenaline; Amidephrine Mesylate; Apraclonidine Hydrochloride; Brimonidine Tartrate; Dapiprazole Hydrochloride; Deterenol Hydrochloride; Dipivefrin; Dopamine Hydrochloride; Ephedrine Sulfate; Epinephrine; Epinephrine Bitartrate; Epinephryl Borate; Esproquin Hydrochloride; Etafedrine Hydrochloride; Hydroxyamphetamine Hydrobromide; Levonordefrin; Mephentermine Sulfate; Metaraminol Bitartrate; Metizoline Hydrochloride; Naphazoline Hydrochloride; Norepinephrine Bitartrate; Oxidopamine; Oxymetazoline Hydrochloride; Phenylephrine Hydrochloride; Phenylpropanolamine Hydrochloride; Phenylpropanolamine Polistirex; Prenalterol Hydrochloride; Propylhexedrine; Pseudoephedrine Hydrochloride; Tetrahydrozoline Hydrochloride; Tramazoline Hydrochloride; Xylometazoline Hydrochloride.

Adrenocortical steroid: Ciprocinonide; Desoxycorticosterone Acetate; Desoxycorticosterone Pivalate; Dexamethasone Acetate; Fludrocortisone Acetate; Flumoxonide; Hydrocortisone Hemisuccinate; Methylprednisolone Hemisuccinate; Naflocort; Procinonide; Timobesone Acetate; Tipredane.

Adrenocortical suppressant: Aminoglutethimide; Trilostane.

Alcohol deterrent: Disulfuram.

Aldosterone antagonist: Canrenoate Potassium; Canrenone; Dicirenone; Mexrenoate Potassium; Prorenoate Potassium; Spironolactone.

Amino acid: Alanine; Arginine; Aspartic Acid; Carnitine; Cysteine Hydrochloride; Cystine; Glycine; Histidine; Isoleucine; Leucine; Lysine; Lysine Acetate; Lysine Hydrochloride; Methionine; Phenylalanine; Proline; Serine; Threonine; Tryptophan; Tyrosine; Valine.

Ammonia detoxicant: Arginine Glutamate; Arginine Hydrochloride.

Amyotrophic lateral sclerosis agents: Riluzole.

Anabolic: Bolandiol Dipropionate; Bolasterone; Boldenone Undecylenate; Bolenol; Bolmantalate; Ethylestrenol; Methenolone Acetate; Methenolone Enanthate; Mibolerone; Nandrolone Cyclotate; Norbolethone; Pizotyline; Quinbolone; Stenbolone Acetate; Tibolone; Zeranol.

Analeptic: Modafinil.

Analgesic: Acetaminophen; Alfentanil Hydrochloride; Aminobenzoate Potassium; Aminobenzoate Sodium; Amidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafenine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Lornoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propiram Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salicylamide; Salicylate Meglumine; Salsalate; Sodium Salicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zomepirac Sodium; Zucapsaicin.

Androgen: Fluoxymesterone; Mesterolone; Methyltestosterone; Nandrolone Decanoate; Nandrolone Phenpropionate; Nisterime Acetate; Oxandrolone; Oxymetholone; Silandrone; Stanozolol; Testosterone; Testosterone Cypionate; Testosterone Enanthate; Testosterone Ketolaurate; Testosterone Phenylacetate; Testosterone Propionate; Trestolone Acetate.

Anesthesia, adjunct to: Sodium Oxybate.

Anesthetic: Aliflurane; Benoxinate Hydrochloride; Benzocaine; Biphenamine Hydrochloride; Bupivacaine Hydrochloride; Butamben; Butamben Picrate; Chloroprocaine Hydrochloride; Cocaine; Cocaine Hydrochloride; Cyclopropane; Desflurane; Dexivacaine; Diamocaine Cyclamate; Dibucaine; Dibucaine Hydrochloride; Dyclonine Hydrochloride; Enflurane; Ether; Ethyl Chloride; Etidocaine; Etoxadrol Hydrochloride; Euprocin Hydrochloride; Fluoroxene; Halothane; Isobutamben; Isoflurane; Ketamine Hydrochloride; Levoxadrol Hydrochloride; Lidocaine; Lidocaine Hydrochloride; Mepivacaine Hydrochloride; Methohexital Sodium; Methoxyflurane; Midazolam Hydrochloride; Midazolam Maleate; Minaxolone; Norflurane; Octodrine; Oxethazaine; Phencyclidine Hydrochloride; Pramoxine Hydrochloride; Prilocalne Hydrochloride; Procaine Hydrochloride; Propanidid; Proparacaine Hydrochloride; Propofol; Propoxycaine Hydrochloride; Pyrrocaine; Risocaine; Rodocaine; Roflurane; Salicyl Alcohol; Sevoflurane; Teflurane; Tetracaine; Tetracaine Hydrochloride; Thiamylal; Thiamylal Sodium; Thiopental Sodium; Tiletamine Hydrochloride; Zolamine Hydrochloride.

Anorectic compounds including: Dexfenfluramine.

Anorexic agents: Aminorex; Amphecloral; Chlorphentermine Hydrochloride; Clominorex; Clortermine Hydrochloride; Diethylpropion Hydrochloride; Fenfluramine Hydrochloride; Fenisorex; Fludorex; Fluminorex; Levamfetamine Succinate; Mazindol; Mefenorex Hydrochloride; Phemnetrazine Hydrochloride; Phentermine; Sibutramine Hydrochloride.

Antagonist: Atipamezole; Atosiban; Bosentan; Cimetidine; Cimetidine Hydrochloride; Clentiazem Maleate; Detirelix Acetate; Devazepide; Donetidine; Etintidine Hydrochloride; Famotidine; Fenmetozole Hydrochloride; Flumazenil; Icatibant Acetate; Icotidine; Isradipine; Metiamide; Nadide; Nalmefene; Naloxone Hydrochloride; Naltrexone; Nilvadipine; Oxilorphan; Oxmetidine Hydrochloride; Oxmetidine Mesylate; Quadazocine Mesylate; Ranitidine; Ranitidine Bismuth Citrate; Ranitidine Hydrochloride; Sufotidine; Teludipine Hydrochloride; Tiapamil Hydrochloride; Tiotidine; Vapiprost Hydrochloride; Zaltidine Hydrochloride.

Anterior pituitary activator: Epimestrol.

Anterior pituitary suppressant: Danazol.

Anthelmintic: Albendazole; Anthelmycin; Bromoxanide; Bunamidine Hydrochloride; Butonate; Cambendazole; Carbantel Lauryl Sulfate; Clioxanide; Closantel; Cyclobendazole; Dichlorvos; Diethylcarbamazine Citrate; Dribendazole; Dymanthine Hydrochloride; Etibendazole; Fenbendazole; Furodazole; Hexylresorcinol; Mebendazole; Morantel Tartrate; Niclosamide; Nitramisole Hydrochloride; Nitrodan; Oxantel Pamoate; Oxfendazole; Oxibendazole; Parbendazole; Piperamide Maleate; piperazine; piperazine Citrate; piperazine Edetate Calcium; Proclonol; Pyrantel Pamoate; Pyrantel Tartrate; Pyrvinium Pamoate; Rafoxanide; Stilbazium Iodide; Tetramisole Hydrochloride; Thiabendazole; Ticarbodine; Tioxidazole; Triclofenol piperazine; Vincofos; Zilantel.

Anti-acne: Adapalene; Erythromycin SaInacedin; Inocoterone Acetate.

Anti-adrenergic: Acebutolol; Alprenolol Hydrochloride; Atenolol; Bretylium Tosylate; Bunolol Hydrochloride; Carteolol Hydrochloride; Celiprolol Hydrochloride; Cetamolol Hydrochloride; Cicloprolol Hydrochloride; Dexpropranolol Hydrochloride; Diacetolol Hydrochloride; Dihydroergotamine Mesylate; Dilevalol Hydrochloride; Esmolol Hydrochloride; Exaprolol Hydrochloride; Fenspiride Hydrochloride; Flestolol Sulfate; Labetalol Hydrochloride; Levobetaxolol Hydrochloride; Levobunolol Hydrochloride; Metalol Hydrochloride; Metoprolol; Metoprolol Tartrate; Nadolol; Pamatolol Sulfate; Penbutolol Sulfate; Phentolamine Mesylate; Practolol; Propranolol Hydrochloride; Proroxan Hydrochloride; Solypertine Tartrate; Sotalol Hydrochloride; Timolol; Timolol Maleate; Tiprenolol Hydrochloride; Tolamolol; Zolertine Hydrochloride.

Anti-allergic: Amlexanox; Astemizole; Azelastine Hydrochloride; Eclazolast; Minocromil Nedocromil Nedocromil Calcium; Nedocromil Sodium; Nivimedone Sodium; Pemirolast Potassium Pentigetide; Pirquinozol; Poisonoak Extract; Probicromil Calcium; Proxicromil; Repirinast; Tetrazolast Meglumine; Thiazinamium Chloride; Tiacrilast; Tiacrilast Sodium; Tiprinast Meglumine; Tixanox.

Anti-amebic: Berythromycin; Bialamicol Hydrochloride; Chloroquine; Chloroquine Hydrochloride; Chloroquine Phosphate; Clamoxyquin Hydrochloride; Clioquinol; Emetine Hydrochloride; Iodoquinol; Paromomycin Sulfate; Quinfamide; Symetine Hydrochloride; Teclozan; Tetracycline; Tetracycline Hydrochloride.

Anti-androgen: Benorterone; Cioteronel; Cyproterone Acetate; Delmadinone Acetate; Oxendolone; Topterone; Zanoterone.

Anti-anemic: Epoetin Alfa; Epoetin Beta; Ferrous Sulfate, Dried; Leucovorin Calcium.

Anti-anginal: Amlodipine Besylate; Amlodipine Maleate; Betaxolol Hydrochloride; Bevantolol Hydrochloride; Butoprozine Hydrochloride; Carvedilol; Cinepazet Maleate; Metoprolol Succinate; Molsidomine; Monatepil Maleate; Primidolol; Ranolazine Hydrochloride; Tosifen; Verapamil Hydrochloride.

Anti-anxiety agent: Adatanserin Hydrochloride; Alpidem; Binospirone Mesylate; Bretazenil; Glemanserin; Ipsapirone Hydrochloride; Mirisetron Maleate; Ocinaplon; Ondansetron Hydrochloride; Panadiplon; Pancopride; Pazinaclone; Serazapine Hydrochloride; Tandospirone Citrate; Zalospirone Hydrochloride.

Anti-arthritic: Lodelaben.

Anti-asthmatic: Ablukast; Ablukast Sodium; Bunaprolast; Cinalukast; Cromitrile Sodium; Cromolyn Sodium; Enofelast; Isamoxole; Ketotifen Fumarate; Levcromakalim; Lodoxamide Ethyl; Lodoxamide Tromethamine; Montelukast Sodium; Ontazolast; Oxarbazole; Oxatomide; Piriprost; Piriprost Potassium; Pirolate; Pobilukast Edamine; Quazolast; Ritolukast; Sulukast; Tiaramide Hydrochloride; Tibenelast Sodium; Tomelukast; Tranilast; Verlukast; Verofylline Zarirlukast.

Anti-atherosclerotic: Mifobate; Timefuronc.

Antibacterial: Acedapsone; Acetosulfone Sodium; Alamecin; Alexidine; Amdinocillin; Amdinocillin Pivoxil; Amicycline; Amifioxacin; Amifloxacin Mesylate; Amikacin; Amikacin Sulfate; Aminosalicylate sodium; Aminosalicylic acid; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Betamicin Sulfate; Biapenem; Biniramycin; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftiroxime Pivoxetil; Ceftizoxime Sodium; Ceftriaxone Sodium; Cefuroxime; Cefuroxime Axetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofingin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isepamicin; Isoconazole; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenanine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifarthiazole; Nifuradene; Nifuraldezone; Nifuratel; Nifuratrone; Nifuirdazil; Nifurimide; Nifurpirinol; Nifurquinazol; Nitrocycline; Nitrofurantoin; Nitromide; Norfloxacin; Novobiocin Sodium; Ofloxacin; Onnetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin.

Anti-cancer supplementary potentiating agents: Amitryptyline; Amoxapine; Amphotericin B; Antiarrhythmic drugs (e.g., Quinidine); Antihypertensive drugs (e.g., Reserpine); Ca++ antagonists (e.g., Verapamil; Calmodulin inhibitors (e.g., Prenylamine; Caroverine); Citalopram); Clomipramine; Clomipramine); Desipramine; Doxepin; Maprotiline); Nifedipine; Nitrendipine; Non-tricyclic anti-depressant drugs (e.g., Sertraline; Nortriptyline; Protriptyline; Sulfoximine) and Multiple Drug Resistance reducing agents such as Cremaphor EL; Thiol depleters (e.g., Buthionine; Trazodone; Tricyclic anti-depressant drugs (e.g., Imipramine; Trifluoroperazine; Trimipramine; Triparanol analogues (e.g., Tamoxifen).

Anticholelithic: Monoctanoin.

Anticholelithogenic: Chenodiol; Ursodiol.

Anticholinergic: Alverinc Citrate; Anisotropine Methylbromide; Atropine; Atropine Oxide Hydrochloride; Atropine Sulfate; Belladonna; Benapryzine Hydrochloride; Benzetimide Hydrochloride; Benzilonium Bromide; Biperiden; Biperiden Hydrochloride; Biperiden Lactate; Clidinium Bromide; Cyclopentolate Hydrochloride; Dexetimide; Dicyclomine Hydrochloride; Dihexyverine Hydrochloride; Domazoline Fumarate; Elantrine; Elucaine; Ethybenztropine; Eucatropine Hydrochloride; Glycopyrrolate; Heteronium Bromide; Homatropine Hydrobromide; Homatropine Methylbromide; Hyoscyamine; Hyoscyamine Hydrobromide; Hyoscyamine Sulfate; Isopropamide Iodide; Mepenzolate Bromide; Methylatropine Nitrate; Metoquizine; Oxybutynin Chloride; Parapenzolate Bromide; Pentapiperium Methylsulfate; Phencarbamide; Poldine Methylsulfate; Proglumide; Propantheline Bromide; Propenzolate Hydrochloride; Scopolamine Hydrobromide; Tematropium Methylsulfate; Tiquinamide Hydrochloride; Tofenacin Hydrochloride; Toquizine; Triampyzine Sulfate; Trihexyphenidyl Hydrochloride; Tropicamide.

Anticoagulant: Ancrod; Ardeparin Sodium; Bivalirudin; Bromindione; Dalteparin Sodium Desirudin; Dicumarol; Lyapolate Sodium; Nafamostat Mesylate; Phenprocoumon; Tinzaparin Sodium; Warfarin Sodium.

Anticoccidal: Maduramicin.

Anticonvulsant: Albutoin; Ameltolide; Atolide; Buramate; Cinromide; Citenamide; Clonazepam; Cyheptamide; Dezinamide; Dimethadione; Divalproex Sodium; Eterobarb; Ethosuximide; Ethotoin; Flurazepam Hydrochloride; Fluzinamide; Fosphenyloin Sodium; Gabapentin; Ilepcimide; Lamotrigine; Magnesium Sulfate; Mephenyloin; Mephobarbital; Methetoin; Methsuximide; Milacemide Hydrochloride; Nabazenil; Nafimidone Hydrochloride; Nitrazepam; Phenacemide; Phenobarbital; Phenobarbital Sodium; Phensuximide; Phenyloin; Phenyloin Sodium; Primidone; Progabide; Ralitoline; Remacemide Hydrochloride; Ropizine; Sabeluzole; Stiripentol; Sulthiame; Topiramate; Trimethadione; Valproate Sodium; Valproic Acid; Vigabatrin; Zoniclezole Hydrochloride; Zonisamide.

Antidepressant: Adinazolam; Adinazolam Mesylate; Alaproclate; Aletamine Hydrochloride; Amedalin Hydrochloride; Amitriptyline Hydrochloride; Aptazapine Maleate; Azaloxan Fumarate; Azepindole; Azipramine Hydrochloride; Bipenamol Hydrochloride; Bupropion Hydrochloride; Butriptyline Hydrochloride; Caroxazone; Cartazolate; Ciclazindol; Cidoxepin Hydrochloride; Cilobamine Mesylate; Clodazon Hydrochloride; Clomipramine Hydrochloride; Cotinine Fumarate; Cyclindole; Cypenamine Hydrochloride; Cyprolidol Hydrochloride; Cyproximide; Daledalin Tosylate; Dapoxetine Hydrochloride; Dazadrol Maleate; Dazepinil Hydrochloride; Desipramine Hydrochloride; Dexamisole; Deximafen; Dibenzepin Hydrochloride; Dioxadrol Hydrochloride; Dothiepin Hydrochloride; Doxepin Hydrochloride; Duloxetine Hydrochloride; Eclanamine Maleate; Encyprate; Etoperidone Hydrochloride; Fantridone Hydrochloride; Fenmetramide; Fezolamine Fumarate; Fluotracen Hydrochloride; Fluoxetine; Fluoxetine Hydrochloride; Fluparoxan Hydrochloride; Gamfexine; Guanoxyfen Sulfate; Imafen Hydrochloride; Imiloxan Hydrochloride; Imipramine Hydrochloride; Indeloxazine Hydrochloride; Intriptyline Hydrochloride; Iprindole; Isocarboxazid; Ketipramine Fumarate; Lofepramine Hydrochloride; Lortalamine; Maprotiline; Maprotiline Hydrochloride; Melitracen Hydrochloride; Minaprine Hydrochloride; Mirtazapine; Moclobemide; Modaline Sulfate; Napactadine Hydrochloride; Napamezole Hydrochloride; Nefazodone Hydrochloride; Nisoxetine; Nitrafudam Hydrochloride; Nomifensine Maleate; Nortriptyline Hydrochloride; Octriptyline Phosphate; Opipramol Hydrochloride; Oxaprotiline Hydrochloride; Oxypertine; Paroxetine; Phenelzine Sulfate; Pirandamine Hydrochloride; Pridefine Hydrochloride; Prolintane Hydrochloride; Protriptyline Hydrochloride; Quipazine Maleate; Rolicyprine; Seproxetine Hydrochloride; Sertraline Hydrochloride; Sulpiride; Suritozole; Tametraline Hydrochloride; Tampramine Fumarate; Tandamine Hydrochloride; Thiazesim Hydrochloride; Thozalinone; Tomoxetine Hydrochloride; Trazodone Hydrochloride; Trebenzomine Hydrochloride; Trimipramine Maleate; Venlafaxine Hydrochloride; Viloxazine Hydrochloride; Zimeldine Hydrochloride; Zometapine.

Antidiabetic: Acetohexamide; Buformin; Butoxamine Hydrochloride; Camighbose; Chlorpropamide; Ciglitazone; Englitazone Sodium; Etoformin Hydrochloride; Gliamilide; Glibomuride; Glicetanile Sodium; Gliflumide; Glipizide; Glucagon; Glyburide; Glyhexamide; Glymidine Sodium; Glyoctamide; Glyparamide; Insulin; Insulin Human; Insulin Human Zinc; Insulin Human Zinc, Extended; Insulin Human, Isophane; Insulin Lispro; Insulin Zinc; Insulin Zinc, Extended; Insulin Zinc, Prompt; Insulin, Dalanated; Insulin, Isophane; Insulin, Neutral; Linogliride; Linogliride Fumarate; Metformin; Methyl Palmoxirate; Palmoxirate Sodium; Pioglitazone Hydrochloride; Pirogliride Tartrate; Proinsulin Human; Seglitide Acetate; Tolazamide; Tolbutamide; Tolpyrramide; Troglitazone; Zopolrestat.

Antidiarrheal: Diphenoxylate Hydrochloride; Methylprednisolone; Metronidazole; Rolgamidine.

Antidiuretic: Argipressin Tannate; Desmopressin Acetate; Lypressin.

Antidote: Dimercaprol; Edrophonium Chloride; Fomepizole; Levoleucovorin Calcium; Methylene Blue; Protamine Sulfate.

Antidyskinetic: Selegiline Hydrochloride.

Anti-emetic: Alosetron Hydrochloride; Batanopride Hydrochloride; Bemesetron; Benzquinamide; Chlorpromazine; Chlorpromazine Hydrochloride; Clebopride; Cyclizine Hydrochloride; Dimenhydrinate; Diphenidol; Diphenidol Hydrochloride; Diphenidol Pamoate; Dolasetron Mesylate; Domperidone; Dronabinol; Flumeridone; Galdansetron Hydrochloride; Granisetron; Granisetron Hydrochloride; Lurosetron Mesylate; Meclizine Hydrochloride; Metoclopramide Hydrochloride; Metopimazine; Prochlorperazine; Prochlorperazine Edisylate; Prochlorperazine Maleate; Promethazine Hydrochloride; Thiethylperazine; Thiethylperazine Malate; Thiethylperazine Maleate; Trimethobenzamide Hydrochloride; Zacopride Hydrochloride.

Anti-epileptic: Felbanate; lamotrigine; Loreclezole; Tolgabide.

Anti-estrogen: Clometherone; Nafoxidine Hydrochloride; Nitromifene Citrate; Raloxifene Hydrochloride; Tamoxifen Citrate; Toremifene Citrate; Trioxifene Mesylate.

Antifibrinolytic: Nafamostat Mesylate.

Antifungal: Acrisorcin; Ambruticin; Azaconazole; Azaserine; Basifungin; Bifonazole; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Itraconazole; Kalafungin; Ketoconazole; Lomoftmgin; Lydimycin; Mepartricin; Miconazole; Miconazole Nitrate; Monensin; Monensin Sodium; Naftifine Hydrochloride; Nifuratel Nifurmerone; Nitralamine Hydrochloride; Nystatin; Octanoic Acid; Orconazole Nitrate; Oxiconazole Nitrate; Oxifingin Hydrochloride; Parconazole Hydrochloride; Partricin; Potassium Iodide; Pyrrolnitrin; Rutamycin; Sanguinarium Chloride; Saperconazole; Selenium Sulfide; Sinefingin; Sulconazole Nitrate; Terbinafine; Terconazole; Thiram; Tioconazole; Tolciclate; Tolindate; Tolnaftate; Triacetin; Triafungin; Undecylenic Acid; Viridofulvin; Zinc Undecylenate; Zinoconazole Hydrochloride.

Antiglaucoma agent: Alprenoxime Hydrochloride; Colforsin; Dipivefrin Hydrochloride; Naboctate Hydrochloride; Pilocarpine; Pimabine.

Antihemorrhagic: Poliglusam.

Antihemorrheologic: Phentoxifylline.

Antihistaminic: Acrivastine; Antazoline Phosphate; Azatadine Maleate; Barmastine; Bromodiphenhydramine Hydrochloride; Brompheniramine Maleate; Carbinoxamine Maleate; Cetirizine Hydrochloride; Chlorpheniramine Maleate; Chlorpheniramine Polistirex; Cirmarizine; Clemastine; Clemastine Fumarate; Closiramine Aceturate; Cycliramine Maleate; Cyclizine; Cyproheptadine Hydrochloride; Dexbrompheniramine Maleate; Dexchlorpheniramine Maleate; Dimethindene Maleate; Diphenhydramine Citrate; Diphenhydramine Hydrochloride; Dorastine Hydrochloride; Doxylamine Succinate; Ebastine; Fexofenadine HCl; Levocabastine Hydrochloride; Loratadine; Mianserin Hydrochloride; Noberastine; Orphenadrine Citrate; Pyrabrom; Pyrilamine Maleate; Pyroxamine Maleate; Rocastine Hydrochloride; Rotoxamine; Tazifylline Hydrochloride; Temelastine; Terfenadine; Tripelennamine Citrate; Tripelennamine Hydrochloride; Triprolidine Hydrochloride.

Antihyperlipidemic: Cholestyramine Resin; Clofibrate; Colestipol Hydrochloride; Crilvastatin; Dalvastatin; Dextrothyroxine Sodium; Fluvastatin Sodium; Gemfibrozil; Lecimibide; Lovastatin; Niacin; Pravastatin Sodium; Probucol; Simvastatin; Tiqueside; Xenbucin.

Antihyperlipoproteinemic: Acifran; Beloxamide; Bezafibrate; Boxidine; Cetaben Sodium; Ciprofibrate; Gemcadiol; Halofenate; Lifibrate; Meglutol; Nafenopin; Pimetine Hydrochloride; Theofibrate; Tibric Acid; Treloxinate.

Antihypertensive: Alfuzosin Hydrochloride; Alipamide; Althiazide; Amiquinsin Hydrochloride; Anaritide Acetate; Atiprosin Maleate; Belfosdil; Bemitradine; Bendacalol Mesylate; Bendroflumethiazide; Benzthiazide; Bethanidine Sulfate; Biclodil Hydrochloride; Bisoprolol; Bisoprolol Fumarate; Bucindolol Hydrochloride; Bupicomide; Buthiazide; Candoxat rilat; Candoxatril; Captopril; Ceronapril; Chlorothiazide Sodium; Cicletanine; Cilazapril; Clonidine; Clonidine Hydrochloride; Clopamide; Cyclopenthiazide; Cyclothiazide; Darodipine; Debrisoquin Sulfate; Delapril Hydrochloride; Diapamide; Diazoxide; Diltiazem Hydrochloride; Diltiazem Malate; Ditekiren; Doxazosin Mesylate; Ecadotril; Enalapril Maleate; Enalaprilat; Enalkiren; Endralazine Mesylate; Epithiazide; Eprosartan; Eprosartan Mesylate; Fenoldopam Mesylate; Flavodilol Maleate; Flordipine; Flosequinan; Fosinopril Sodium; Fosinoprilat; Guanabenz; Guanabenz Acetate; Guanacline Sulfate; Guanadrel Sulfate; Guancvdine; Guanethidine Monosulfate; Guanethidine Sulfate; Guanfacine Hydrochloride; Guanisoquin Sulfate; Guanoclor Sulfate; Guanoctine Hydrochloride; Guanoxabenz; Guanoxan Sulfate; Guanoxyfen Sulfate; Hydralazine Hydrochloride; Hydralazine Polistirex; Hydroflumethiazide; Indacrinone Indapamide; Indolapril Hydrochloride; Indoramin; Indoramin Hydrochloride; Indorenate Hydrochloride; Lacidipine; Leniquinsin; Lisinopril; Lofexidine Hydrochloride; Losartan Potassium; Losulazine Hydrochloride; Mebutamate; Mecamylamine Hydrochloride; Medroxalol; Medroxalol Hydrochloride; Methalthiazide Methyclothiazide Methyldopa; Methyldopate Hydrochloride; Metipranolol; Metolazone Metoprolol Fumarate; Metyrosine; Minoxidil; Muzolimine; Nebivolol; Nifidipine; Oformine; Pargyline Hydrochloride; Pazoxide; Pelanserin Hydrochloride; Perindopril Erbumine; Phenoxybenzamine Hydrochloride; Pinacidil; Pivopril; Polythiazide; Prazosin Hydrochloride; Prizidilol Hydrochloride; Quinapril Hydrochloride; Quinaprilat; Quinazosin Hydrochloride; Quinelorane Hydrochloride; Quinpirole Hydrochloride; Quinuclium Bromide; Ramipril; Rauwolfia Serpentina; Reserpine; Saprisartan Potassium; Saralasin Acetate; Sodium Nitroprusside; Sulfinalol Hydrochloride; Tasosartan; Temocapril Hydrochloride; Terazosin Hydrochloride; Terlakiren; Tiamenidine; Tiamenidine Hydrochloride; Ticrynafen; Tinabinol; Tiodazosin; Tipentosin Hydrochloride; Trichlormethiazide; Trimazosin Hydrochloride; Trimethaphan Camsylate; Trimoxamine Hydrochloride; Tripamide; Xipamide; Zankiren Hydrochloride; Zofenoprilat Arginine.

Antihypotensive: Ciclafrine Hydrochloride; Midodrine Hydrochloride.

Anti-infective: Acyclovir; Difloxacin Hydrochloride; Integrase Inhibitors of HIV and other retroviruses; Lauryl Isoquinolinium Bromide; Moxalactam Disodium; Ornidazole; Pentisomicin; Protease inhibitors of HIV and other retroviruses; Sarafloxacin Hydrochloride.

Anti-infective, topical: Alcohol; Aminacrine Hydrochloride; Benzethonium Chloride; Bithionolate Sodium; Bromchlorenone; Carbamide Peroxide; Cetalkonium Chloride; Cetylpyridinium Chloride; Chlorhexidine Hydrochloride; Domiphen Bromide; Fenticlor; Fludazonium Chloride; Fuchsin, Basic; Furazolidone; Gentian Violet; Halquinols; Hexachlorophene; Hydrogen Peroxide; Ichthammol; Imidecyl Iodine; Iodine; Isopropyl Alcohol; Mafenide Acetate; Meralcin Sodium; Mercufenol Chloride; Mercury, Ammoniated; Methylbenzethonium Chloride; Nitrofarazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal; Troclosene Potassium.

Anti-inflammatory: Alclofenac; Alclometasone Dipropionate; Algestone Acetonide; Alpha Amylase; Amcinafal; Amcinafide; Amfenac Sodium; Amiprilose Hydrochloride; Anakinra; Anitrazafen; Apazone; Balsalazide Disodium; Bendazac; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Difluprednate; Diftalone; Dimethyl Sulfoxide; Drocinonide; Endrysone; Enlimomab; Enolicam Sodium; Etodolac; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Fluocortin Butyl; Fluorometholone Acetate; Fluquazone; Fluretofen; Fluticasone Propionate; Furaprofen; Furobufen; Halcinonide; Halobetasol Propionate; Halopredone Acetate; Ibuprofen; Ibuprofen Aluminum; Ibuprofen Piconol; Ilonidap; indomethacin Sodium; Indomethacin; Indoprofen Indoxole; Intrazole; Isoflupredone Acetate; Isoxepac; Isoxicam; Ketoprofen; Lomoxicam; Loteprednol Etabonate; Meclofenamate Sodium; Meclofenamic Acid; Meclorisonc Dibutyrate; Mesalamine; Meseclazone; Methylprednisolone Suleptanate; Morniflumate; Nabumetone; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Piroxicam; Piroxicam Cinnamate; Pirprofen; Prednazate; Prednisolone Sodium Phosphate; Prifelone; Prodolic Acid; Proquazone; Rimexolone; Romazarit; Salnacedin; Seclazone; Sermetacin; Sudoxicam; Sulindac; Suprofen; Talniflumate; Tenidap; Tenidap Sodium; Tenoxicam; Tesicam; Tesimide; Tixocortol Pivalate; Tolmetin; Tolmetin Sodium; Triclonide; Triflumidate; Zidometacin.

Antikeratinizing agent: Doretinel; Linarotene; Pelretin.

Antimalarial: Amodiaquine Hydrochloride; Amquinate; Artefiene; Chloroquine; Chloroquine Hydrochloride; Cycloguanil Pamoate; Enpiroline Phosphate; Halofantrine Hydrochloride; Hydroxychloroquine Sulfate; Mefloquine Hydrochloride; Menoctone; Primaquine Phosphate; Pyrimethamine; Quinine Sulfate; Tebuquine.

Antimicrobial: Aztreonam; Chlorhexidine Gluconate; Imidurea; Lycetamine; Nibroxane; Pirazmonam Sodium; Propionic Acid; Pyrithione Sodium; Tigemonam Dicholine.

Antimigraine: Naratriptan Hydrochloride; Sergolexole Maleate; Sumatriptan Succinate; Zatosetron Maleate.

Antimitotic: Podofilox.

Antimycotic: Amorolfine.

Antinauseant: Buclizine Hydrochloride; Cyclizine Lactate.

Antineoplastic: Acivicin; Aclarubicin; Acodazole Hydrochloride; Acronine; Adozelesin; Aldesleukin; Altretamine; Ambomycin; Ametantrone Acetate; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cisplatin; Cladribine; Crisnatol Mesylate; Cyclophosphamide; Cytarabine; Dacarbazine; Dactinomycin; Daunorubicin Hydrochloride; Decitabine; Dexorinaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n3; Interferon Alfa-n1; Interferon Beta-I a; Interferon Gamma-I b; Iproplatin; Irinotecan Hydrochloride; Isotretinoin; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamvcin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Prednimustine; Procarbazine Hydrochloride; Puromycin; Puromycin Hydrochloride; Pyrazoftrin; Riboprine; Rogletimide; Safingol Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofarin; Tirapazamine; Topotecan Hydrochloride; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Anti-neoplastic compounds, additional: 20-epi-1,25 Dihydroxyvitamin D3; 5-Ethynyluracil; Abiraterone; Acylfulvene; Adecypenol; ALL-TK Antagonists; Ambamustine; Amidox; Amifostine; Aminolevulinic Acid; Amrubicin; Anagrelide; Andrographolide; Angiogenesis Inhibitors; Antagonist D; Antagonist G; Antarelix; Antiandrogen, Prostatic Carcinoma; Anti-Dorsalizing Morphogenetic Protein-1; Antiestrogen; Antineoplaston; Antisense Oligonucleotides; Aphidicolin Glycinate; Apoptosis Gene Modulators; Apoptosis Regulators; Apurinic Acid; Ara-CDP-DL-PTBA; Arginine Deaminase; Asulacrine; Atamestane; Atrimustine; Axinastatin 1; Axinastatin 2; Axinastatin 3; Azasetron; Azatoxin; Azatyrosine; Baccatin III Derivatives; Balanol; BCR/ABL Antagonists; Benzochlorins; Benzoylstaurosporine; Beta Lactam Derivatives; Beta-Alethine; Betaclamycin B; Betulinic Acid; bFGF Inhibitor; Bisantrene; Bisaziridinylspermine; Bisnafide; Bistratene A; Breflate; Budotitane; Buthionine Sulfoximine; Calcipotriol; Calphostin C; Camptothecin Derivatives; Canarypox IL-2; Capecitabine; Carboxamide-Amino-Triazole; Carboxyamidotriazole; CaRest MI; CARN 700, Cartilage Derived Inhibitor; Casein Kinase Inhibitors (ICOS); Castanospermine; Cecropin B; Cetrorelix; Chlorins; Chloroquinoxaline Sulfonamide; Cicaprost; Cis-Porphyrin; Clomifene analogues; Collismycin A; Collismycin B; Combretastatin A4; Combretastatin Analogue; Conagenin; Crambescidin 816; Crisnatol; Cryptophycin 8; Cryptophycin A Derivatives; Curacin A; Cyclopentanthraquinones; Cycloplatam; Cypemycin; Cytarabine Ocfosfate; Cytolytic Factor; Cytostatin; Dacliximab; Dehydrodidenmin B; Dexifosfamide; Dexverapamil; Didemnin B; Didox; Diethylnorspennine; Dihydro Azacytidine; Dihydrotaxol, 9-; Dioxamycin; Diphenyl Spiromustine; Docosanol; Dolasetron; Doxifluridine; Duocarmycin SA; Ebselen; Ecomustine; Edelfosine; Edrecolomab; Eflomithine; Elemene; Emitefur; Epirubicin; Estramustine Analogue; Estrogen Agonists; Estrogen Antagonists; Exemestane; Fadrozole; Fiezelastine; Flavopiridol; Fluasterone; Fludarabine; Fluorodaunorunicin Hydrochloride; Forfenimex; Formestane; Fostriecin; Fotemustine; Gadolinium Texaphyrin; Gallium Nitrate; Galocitabine; Ganirelix; Gelatinase Inhibitors; Glutathione Inhibitors; Hepsulfam; Heregulin; Hexamethylene Bisacetamide; Hypericin; Ibandronic acid; Idarubicin; Idoxifene; Idramantone; Ilomastat; Imidazoacridones; Immunostimulant Peptides; Insulin-Like Growth Factor-1 Receptor Inhibitor; Interferon Agonists; Interferons; Interleukins; Iobenguane; Iododoxorubicin; Ipomeanol, 4-; Irinotecan; Iroplact; Irsogladine; Isobengazole; Isohomohalicondrin B; Itasetron; Jasplakinolide; Kahalalide F; Lamellarin-N Triacetate; Lanreotide; Leinamycin; Lentinan Sulfate; Leptolstatin; Leukemia Inhibiting Factor; Leukocyte Alpha Interferon; Leuprolide+Estrogen+Progesterone; Leuprorelin; Levamisole; Liarozole; Linear Polyamine Analogue; Lipophilic Disaccharide Peptide; Lipophilic Platinum Compounds; Lissoclinamide 7; Lobaplatin; Lombricine; Lometrexol; Lonidamine; Losoxantrone; Lurtotecan; Lutetium Texaphyrin; Lysofylline; Lytic Peptides; Maitansine; Mannostatin A; Marimastat; Maspin; Matrilysin Inhibitors; Matrix Metalloproteinase Inhibitors; Merbarone; Meterelin; Methioninase; Metoclopramide; MIF Inhibitor; Mifepristone; Miltefosine; Mirimostim; Mismatched Double Stranded RNA; Mitoguazone; Mitolactol; Mitomycin analogues; Mitonafide; Mitotoxin Fibroblast Growth Factor-Saporin; Mitoxantrone; Mofarotene; Monoclonal Antibody, Human Chorionic Gonadotrophin; Monophosphoryl Lipid A+Myobacterium Cell Wall Sk; Mopidamol; Multiple Drug Resistance Gene Inhibitor; Multiple Tumor Suppressor 1-Based Therapy; Mustard Anticancer Agent; Mycaperoxide B; Mycobacterial Cell Wall Extract; Myriaporone; N-Acetyldinaline; Nafarelin; Nagrestip; Naloxone+Pentazocine; Napavin; Naphterpin; Nartograstim; Nedaplatin; Nemorubicin; Neridronic Acid; Neutral Endopeptidase; Nilutamide; Nisamycin; Nitric Oxide Modulators; Nitroxide Antioxidant; Nitrullyn; N-Substituted Benzamides; O6-Benzylguanine; Okicenone; Oligonucleotides; Onapristone; Ondansetron; Oracin; Oral Cytokine Inducer; Osaterone; Oxaliplatin; Oxaunomycin; Paclitaxel Analogues; Paclitaxel Derivatives; Palauamine; Palmitoylrhizoxin; Pamidronic Acid; Panaxytriol; Panomifene; Parabactin; Pazelliptine; Peldesine; Pentostatin; Pentrozole; Perflubron; Perillyl Alcohol; Phenazinomycin; Phenylacetate; Phosphatase Inhibitors; Picibanil; Pilocarpine Hydrochloride; Pirarubicin; Piritrexim; Placetin A; Placetin B; Plasminogen Activator Inhibitor; Platinum Complex; Platinum Compounds; Platinum-Triamine Complex; Propyl Bis-Acridone; Prostaglandin J2; Proteasome Inhibitors; Protein A-Based Immune Modulator; Protein Kinase C Inhibitor; Protein Kinase C Inhibitors, Microalgal; Protein Tyrosine Phosphatase Inhibitors; Purine Nucleoside Phosphorylase Inhibitors; Purpurins; Pyrazoloacridine; Pyridoxylated Hemoglobin Polyoxyethylene Conjugate; Raf Antagonists; Raltitrexed; Ramosetron; Ras Farnesyl Protein Transferase Inhibitors; Ras Inhibitors; Ras-GAP Inhibitor; Retelliptine Demethylated; Rhenium, Re 186 Etidronate; Rhizoxin; Ribozymes; R1H Retinamide; Rohitukine; Romurtide; Roquinimex; Rubiginone B1; Ruboxyl; Safingol; Saintopin; SarCNU; Sarcophytol A; Sdi 1 Mimetics; Senescence Derived Inhibitor 1; Sense Oligonucleotides; Signal Transduction Inhibitors; Signal Transduction Modulators; Single Chain Antigen Binding Protein; Sizofuran; Sobuzoxane; Sodium Borocaptate; Sodium Phenylacetate; Solverol; Somatomedin Binding Protein; Sonermin; Sparfosic Acid; Spicamycin D; Splenopentin; Spongistatin 1; Squalamine; Stem Cell Inhibitor; Stem-Cell Division Inhibitors; Stipiamide; Stromelysin Inhibitors; Sulfinosine; Superactive Vasoactive Intestinal Peptide Antagonist; Suradista; Suramin; Swainsonine; Synthetic Glycosaminoglycans; Tallimustine; Tamoxifen Methiodide; Tauromustine; Tellurapyrylium; Telomerase Inhibitors; Temozolomide; Tetrachlorodecaoxide; Tetrazomine; Thaliblastine; Thalidomide; Thiocoraline; Thrombopoietin; Thrombopoietin Mimetic; Thymalfasin; Thymopoietin Receptor Agonist; Thymotrinan; Thyroid Stimulating Hormone; Tin Ethyl Etiopurpurin; Titanocene Dichloride; Topotecan; Topsentin; Toremifene; Totipotent Stem Cell Factor; Translation Inhibitors; Triacetyluridine; Triciribine; Tropisetron; Turosteride; Tyrosine Kinase Inhibitors; Tyrphostins; UBC Inhibitors; Ubenimex; Urogenital Sinus-Derived Growth Inhibitory Factor; Urokinase Receptor Antagonists; Variolin B; Vector system, Erythrocyte Gene Therapy; Velaresol; Veramine; Verdins; Vinorelbine; Vinxaltine; Vitaxin; Zilascorb; Zinostatin Stimalamer.

Antineutropenic: Filgrastim; Lenograstim; Molgramostim; Regramostim; Sargramostim.

Antiobsessional agent: Fluvoxamine Maleate.

Antiparasitic: Abamectin; Clorsulon; Ivermectin.

Antiparkinsonian: Benztropine Mesylate; Biperiden; Biperiden Hydrochloride; Biperiden Lactate; Carbidopa-Levodopa; Carmantadine; Ciladopa Hydrochloride; Dopamantine; Ethopropazine Hydrochloride; Lazabemide; Levodopa; Lometraline Hydrochloride; Mofegiline Hydrochloride; Naxagolide Hydrochloride; Pareptide Sulfate; Procyclidine Hydrochloride; Ropinirole Hydrochloride; Tolcapone.

Antiperistaltic: Difenoximide Hydrochloride; Difenoxin; Fluperamide; Lidamidine Hydrochloride; Loperamide Hydrochloride; Malethamer; Nufenoxole; Paregoric.

Antipneumocystic: Atovaquone.

Antiproliferative agent: Piritrexim Isethionate.

Antiprostatic hypertrophy: Sitogluside.

Antiprotozoal: Amodiaquine; Azanidazole; Banmidazole; Camidazole; Chlortetracycline Bisulfate Chlortetracycline Hydrochloride; Flubendazole; Flunidazole; Halofuginone Hydrobromide; Imidocarb Hydrochloride; Ipronidazole; Misonidazole; Moxnidazole; Nitarsone; Ronidazole; Sulnidazole; Tinidazole.

Antipruritic: Methdilazine; Methdilazine Hydrochloride; Trimeprazine Tartrate.

Antipsoriatic: Acitretin; Anthralin; Azaribine; Calcipotriene; Cycloheximide; Enazadrem Phosphate; Etretinate; Liarozole Fumarate; Lonapalene; Tepoxalin.

Antipsychotic: Acetophenazine Maleate; Alentemol Hydrobromide; Alpertine; Azaperone; Batelapine Maleate; Benperidol; Benzindopyrine Hydrochloride; Brofoxine; Bromperidol; Bromperidol Decanoate; Butaclamol Hydrochloride; Butaperazine; Butaperazine Maleate; Carphenazine Maleate; Carvotroline Hydrochloride; Chlorprothixene; Cinperene; Cintriamide; Clomacran Phosphate; Clopenthixol; Clopimozide; Clopipazan Mesylate; Cloroperone Hydrochloride; Clothiapine; Clothixamide Maleate; Clozapine; Cyclophenazine Hydrochloride; properidol; Etazolate Hydrochloride; Fenimide; Flucindole; Flumezapine; Fluphenazine Decanoate; Fluphenazine Enanthate; Fluphenazine Hydrochloride; Fluspiperone; Fluspirilene; Flutroline; Gevotroline Hydrochloride; Halopemide; Haloperidol; Haloperidol Decanoate; Iloperidone; Imidoline Hydrochloride; Lenperone; Mazapertine Succinate; Mesoridazine; Mesoridazine Besylate; Metiapine; Milenperone; Milipertine; Molindone Hydrochloride; Naranol Hydrochloride; Neflumozide Hydrochloride; Ocaperidone; Olanzapine; Oxiperomide; Penfluridol; Pentiapine Maleate; Perphenazine; Pimozide; Pinoxepin Hydrochloride; Pipamperone; Piperacetazine; Pipotiazine Palmitate; Piquindone Hydrochloride; Promazine Hydrochloride; Remoxipride; Remoxipride Hydrochloride; Rimcazole Hydrochloride; Seperidol Hydrochloride; Sertindole; Setoperone; Spiperone; Thioridazine; Thioridazine Hydrochloride; Thiothixene; Thiothixene Hydrochloride; Tioperidone Hydrochloride; Tiospirone Hydrochloride; Trifluoperazine Hydrochloride; Trifluperidol; Triflupromazine; Triflupromazine Hydrochloride; Ziprasidone Hydrochloride.

Antirheumatic: Auranofin; Aurothioglucose; Bindarit; Lobenzarit Sodium; Phenylbutazone; Pirazolac; Prinomide Tromethamine; Seprilose.

Antischistosomal: Becanthone Hydrochloride; Hycanthone; Lucanthone Hydrochloride; Niridazole; Oxamniquine; Pararosaniline Pamoate; Teroxalene Hydrochloride.

Antiseborrheic: Chloroxine; Piroctone; Piroctone Olamine; Resorcinol Monoacetate.

Antisecretory: Arbaprostil; Deprostil; Fenoctimine Sulfate; Octreotide; Octreotide Acetate; Omeprazole Sodium; Rioprostil; Trimoprostil.

Antispasmodic: Stilonium Iodide; Tizanidine Hydrochloride.

Antithrombotic: Anagrelide Hydrochloride; Dalteparin Sodium; Danaparoid Sodium; Dazoxiben Hydrochloride; Efegatran Sulfate; Enoxaparin Sodium; Ifetroban; Ifetroban Sodium; Trifenagrel.

Antitussive: Benzonatate; Butamirate Citrate; Chlophedianol Hydrochloride; Codeine Polistirex; Codoxime; Dextromethorphan; Dextromethorphan Hydrobromide; Dextromethorphan Polistirex; Ethyl Dibunate; Guaiapate; Hydrocodone Bitartrate; Hydrocodone Polistirex; Levopropoxyphene Napsylate; Noscapine; Pemerid Nitrate; Pipazethate; Suxemerid Sulfate.

Anti-ulcerative: Aceglutamide Aluminum; Cadexomer Iodine; Cetraxate Hydrochloride; Enisoprost; Isotiquimide; Lansoprazole; Lavoltidine Succinate; Misoprostol; Nizatidine; Nolinium Bromide; Pantoprazole; Pifamine; Pirenzepine Hydrochloride; Rabeprazole Sodium; Remiprostol; Roxatidine Acetate Hydrochloride; Sucralfate; Sucrosofate Potassium; Tolimidone.

Anti-urolithic: Cysteamine; Cysteamine Hydrochloride; Tricitrates.

Antiviral: Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime.

Appetite suppressant: Dexfenfluramine Hydrochloride; Phendimetrazine Tartrate; Phentermine Hydrochloride.

Benign prostatic hyperplasia therapy agent: Tamsulosin Hydrochloride.

Blood glucose regulators: Acetohexamide and Glipizide; Chloropropamide; Human insulin.

Bone resorption inhibitor: Alendronate Sodium; Etidronate Disodium; Pamidronate Disodium.

Bronchodilator: Albuterol; Albuterol Sulfate; Azanator Maleate; Bamifylline Hydrochloride; Bitolterol Mesylate; Butaprost; Carbuterol Hydrochloride; Clorprenaline Hydrochloride; Colterol Mesylate; Doxaprost; Doxofylline; Dyphylline; Enprofylline; Ephedrine; Ephedrine Hydrochloride; Fenoterol; Fenprinast Hydrochloride; Guaithylline; Hexoprenaline Sulfate; Hoquizil Hydrochloride; Ipratropium Bromide; Isoetharine; Isoetharine Hydrochloride; Isoetharine Mesylate; Isoproterenol Hydrochloride; Isoproterenol Sulfate; Metaproterenol Polistirex; Metaproterenol Sulfate; Nisbuterol Mesylate; Oxtriphylline; Picumeterol Fumarate; Piquizil Hydrochloride; Pirbuterol Acetate; Pirbuterol Hydrochloride; Procaterol Hydrochloride; Pseudoephedrine Sulfate; Quazodine; Quinterenol Sulfate; Racepinephrine; Racepinephrine Hydrochloride; Reproterol Hydrochloride; Rimiterol Hydrobromide; Salmeterol; Salmeterol Xinafoate; Soterenol Hydrochloride; Sulfonterol Hydrochloride; Suloxifen Oxalate; Terbutaline Sulfate; Theophylline; Xanoxate Sodium; Zindotrine; Zinterol Hydrochloride.

Carbonic anhydrase inhibitor: Acetazolamide; Acetazolamide Sodium; Dichlorphenamide; Dorzolamide Hydrochloride; Methazolamide; Sezolamide Hydrochloride.

Cardiac depressant: Acecamide Hydrochloride; Acetylcholine Chloride; Actisomide; Adenosine; Amiodarone; Aprindine; Aprindine Hydrochloride; Artilide Fumarate; Azimilide Dihydrochloride; Bidisomide; Bucamide Maleate; Bucromarone; Capobenate Sodium; Capobenic Acid; Cifenline; Cifenline Succinate; Clofilium Phosphate; Disobutamide; Disopyramide; Disopyramide Phosphate; Dofetilide; Drobuline; Edifolone Acetate; Emilium Tosylate; Encamide Hydrochloride; Flecamide Acetate; Ibutilide Fumarate; Indecamide Hydrochloride; Ipazilide Fumarate; Lorajmine Hydrochloride; Lorcamide Hydrochloride; Meobentine Sulfate; Mexiletine Hydrochloride; Modecamide; Moricizine; Oxiramide; Pirmenol Hydrochloride; Pirolazamide; Pranolium Chloride; Procainamide Hydrochloride; Propafenone Hydrochloride; Pyrinoline; Quindonium Bromide; Quinidine Gluconate; Quinidine Sulfate; Recainam Hydrochloride; Recainam Tosylate; Risotilide Hydrochloride; Ropitoin Hydrochloride; Sematilide Hydrochloride; Suricamide Maleate; Tocamide; Tocamide Hydrochloride; Transcamide.

Cardioprotectant: Dexrazoxane; Draflazine.

Cardiotonic: Actodigin; Aminone; Bemoradan; Butopamine; Carbazeran; Carsatrin Succinate; Deslanoside; Digitalis; Digitoxin; Digoxin; Dobutanine; Dobutanine Hydrochloride; Dobutamine Lactobionate; Dobutamine Tartrate; Enoximone; Imazodan Hydrochloride; Indolidan; Isomazole Hydrochloride; Levdobutamine Lactobionate; Lixazinone Sulfate; Medorinone; Milrinone; Pelrinone Hydrochloride; Pimobendan; Piroximone; Prinoxodan; Proscillaridin; Quazinone; Tazolol Hydrochloride; Vesnarinone.

Cardiovascular agent: Dopexamine; Dopexamine Hydrochloride.

Cerebral ischemia agents: Dextrorphan Hydrochloride.

Choleretic: Dehydrocholic Acid; Fencibutirol; Hymecromone; Piprozolin; Sincalide; Tocamphyl.

Cholinergic: Aceclidine; Bethanechol Chloride; Carbachol; Demecarium Bromide; Dexpanthenol; Echothiophate Iodide; Isofluorophate; Methacholine Chloride; Neostiamine Methylsulfate; Neostigmine Bromide; Physostigmine; Physostigmine Salicylate; Physostigmine Sulfate; Pilocarpine Nitrate; Pyridostigmine Bromide.

Cholinergic agonist: Xanomeline; Xanomeline Tartrate.

Cholinesterase Deactivator: Obidoxime Chloride; Pralidoxime Chloride; Pralidoxime Iodide; Pralidoxime Mesylate.

Coccidiostat: Arprinocid; Narasin; Semduramicin; Semduramicin Sodium.

Cognition adjuvant: Ergoloid Mesylates; Piracetam; Pramiracetam Hydrochloride; Pramiracetam Sulfate; Tacrine Hydrochloride.

Cognition enhancer: Besipirdine Hydrochloride; Linopirdine; Sibopirdine.

Contrast Media: Barium Sulfate; Diatrizoate Sodium; Erythrosine Sodium; lopanoic Acid; Ipodate Calcium; Metyrapone; Tyropanoate Sodium.

Diagnostic aid: Aminohippurate Sodium; Anazolene Sodium; Arclofenin; Bentiromide; Benzylpenicilloyl Polylysine; Butedronate Tetrasodium; Butilfenin; Coccidioidin; Corticorelin Ovine Triflutate; Corticotropin Zinc Hydroxide; Corticotropin, Repository; Diatrizoate Meglumine; Diatrizoic Acid; Diphtheria Toxin for Schick Test; Disofenin; Ethiodized Oil; Etifenin; Exametazime; Ferristenc; Ferumoxides; Ferumoxsil; Fluorescein; Fluorescein Sodium; Gadobenate Dimeglumine; Gadodiamide; Gadopentetate Dimegiumine; Gadoteridol; Gadoversetamide; Histoplasmin; Impromidine Hydrochloride; Indigotindisulfonate Sodium; Indocyanine Green; Iobenguane Sulfate I 123; Iobenzamic Acid; Iocarmate Meglumine; Iocarmic Acid; Iocetamic Acid; Iodamide; lodamide Megiumine; Iodipamide Meglumine; Iodixanol; Iodoxamate Meglumine; Iodoxamic Acid; loglicic Acid; loglucol; Ioglucomide; Ioglycamic Acid; Iogulamide; lohexyl; lomeprol; Iopamidol; Iopentol; Iophendylate; Ioprocemic Acid; Iopronic Acid; Iopydol; lopydone; Iosefamic Acid; Ioseric Acid; losulamide Meglumine; Iosumetic Acid; lotasul; lotetric Acid; lothalamate Meglumine; Iothalamate Sodium; Iothalamic Acid; Iotrolan; lotroxic Acid; Ioversol; Ioxagiate Sodium; Ioxaglate Meglumine; Ioxaglic Acid; Ioxilan; Ioxotrizoic Acid; Ipodate Sodium; Iprofenin; Isosulfan Blue; Leukocyte Typing Serum; Lidofenin; Mebrofenin; Meglumine; Metrizamide; Metrizoate Sodium; Metyrapone Tartrate; Mumps Skin Test Antigen; Pentetic Acid; Propyliodone; Quinaldine Blue; Schick Test Control; Sermorelin Acetate; Sodium Iodide I 123; Sprodiamide; Stannous Pyrophosphate; Stannous Sulfur Colloid; Succimer; Teriparatide Acetate; Tetrofosmin; Tolbutamide Sodium; Tuberculin; Xylose.

Diuretic: Ambuphylline; Ambuside; Amiloride Hydrochloride; Azolimine; Azosemide; Brocrinat; Bumetamide; Chlorothiazide; Chlorthalidone; Clazolimine; Clorexolone; Ethacrynate Sodium; Ethacrynic Acid; Etozolin; Fenquizone; Furosemide; Hydrochlorothiazide; Isosorbide; Mannitol Mefruside; Ozolinone; Piretamide; Spiroxasone; Torsemide; Triamterene; Triflocin; Urea.

Dopaminergic agent: Ibopamine.

Ectoparasiticide: Nifluridide; Permethrin.

Emetic: Apomorphine Hydrochloride.

Enzyme inhibitor: 30 Polignate Sodium; Acetohydroxamic Acid; Alrestatin Sodium; Aprotinin; Benazepril Hydrochloride; Benazeprilat; Benurestat; Bromocriptine; Bromocriptine Mesylate; Cilastatin Sodium; Fluorofamide; Lergotrile; Lergotrile Mesylate; Levcycloserine; Libenzapril; Pentopril; Pepstatin; Perindopril; Sodium Amylosulfate; Sorbinil; Spirapril Hydrochloride; Spiraprilat; Taleranol; Teprotide; Tolfamide; Zofenopril Calcium.

Estrogen: Chlorotrianisene; Dienestrol; Diethylstilbestrol; Diethylstilbestrol Diphosphate; Equilin; Estradiol; Estradiol Cypionate; Estradiol Enanthate; Estradiol Undecylate; Estradiol Valerate; Estrazinol Hydrobromide; Estriol; Estrofurate; Estrogens, Conjugated; Estrogens, Esterified; Estrone; Estropipate; Ethinyl Estradiol; Fenestrel; Mestranol; Nylestriol; Quinestrol.

Fibrinolytic: Anistreplase; Bisobrin Lactate; Brinolase.

Free oxygen radical scavenger: Pegorgotein.

Gastric Acid Suppressant: Omeprazole.

Gastrointestinal Motility agents: Cisapride.

Glucocorticoid: Amcinonide; Beclomethasone Dipropionate; Betamethasone; Betamethasone Acetate; Betamethasone Benzoate; Betamethasone Dipropionate; Betamethasone Sodium Phosphate; Betamethasone Valerate; Carbenoxolone Sodium; Clocortolone Acetate; Clocortolone Pivalate; Cloprednol; Corticotropin; Cortisone Acetate; Cortivazol; Descinolone Acetonide; Dexamethasone; Dexamethasone Sodium Phosphate; Diflucortolone; Diflucortolone Pivalate; Flucloronide; Flumethasone; Flumethasone Pivalate; Flunisolide; Fluocinolone Acetonide; Fluocinonide; Fluocortolone; Fluocortolone Caproate; Fluorometholone; Fluperolone Acetate; Fluprednisolone; Fluprednisolone Valerate; Flurandrenolide; Formocortal; Hydrocortisone; Hydrocortisone Acetate; Hydrocortisone Buteprate; Hydrocortisone Butyrate; Hydrocortisone Sodium Phosphate; Hydrocortisone Sodium Succinate; Hydrocortisone Valerate; Medrysone; Methylprednisolone Acetate; Methylprednisolone Sodium Phosphate; Methylprednisolone Sodium Succinate; Nivazol; Paramethasone Acetate; Prednicarbate; Prednisolone; Prednisolone Acetate; Prednisolone Hemisuccinate; Prednisolone Sodium Succinate; Prednisolone Tebutate; Prednisone; Prednival; Ticabesone Propionate; Tralonide; Triamcinolone; Triamcinolone Acetonide; Triamcinolone Acetonide Sodium; Triamcinolone Diacetate; Triamcinolone Hexacetonide.

Gonad-stimulating principle: Buserelin Acetate; Clomiphene Citrate; Ganirelix Acetate; Gonadorelin Acetate; Gonadorelin Hydrochloride; Gonadotropin, Chorionic; Menotropins.

Hair growth stimulant: Aminocaproic Acid; Minoxidil Hemostatic; Oxamarin Hydrochloride; Sulmarin; Thrombin; Tranexamic Acid.

Hormone: 17 Alpha Dihydroequilenin; 17 Alpha Dihydroequilin; 17 Alpha Estradiol; 17 Beta Estradiol; 17 Hydroxy Progesterone; Androstenedione; Clomiphene; Cosyntropin; Dehydroepiandrosterone; Dihydroestosterone; Equilenin; Ethyndiol; Follicle Regulatory Protein; Follicle Stimulating Hormone; Folliculostatin; Gonadoctrinins; Gonadorelin; Gonadotropins; Han Memopausal Gonadotropins; Human Chorionic Gonadotropin; Insulin Growth Factor; Leuprolide; Levonorgestrel; Luteinizing hormone; Luteinizing Hormone Releasing Hormone and Analogs; Medroxyprogesterone; Megestrol; Metogest; Norethindrone; Norethynodrel; Norgestrel; Oocyte Maturation Inhibitor; Oxytocin; Pituitary, Posterior; Progesterone; Relaxin; Seractide Acetate; Somalapor; Somatrem; Somatropin; Somenopor; Somidobove; Tamoxifen; Urofollitropin; Vasopressin.

Hypocholesterolemic: Lifibrol.

Hypoglycemic: Darglitazone Sodium; Glimepiride.

Hypolipidemic: Azalanstat Dihydrochloride; Colestolone; Surfomer; Xenalipin.

Hypotensive: Viprostol.

Immunizing agent: Antirabies Serum; Antivenin; Antivenin (Crotalidae) Polyvalent; BCG Vaccine; Botulism Antitoxin; Cholera Vaccine; Diphtheria Antitoxin; Diphtheria Toxoid; Diphtheria Toxoid Adsorbed; Globulin, Immune; Hepatitis B Immune Globulin; Hepatitis B Virus Vaccine Inactivated; Influenza Virus Vaccine; Measles Virus Vaccine Live; Meningococcal Polysaccharide Vaccine Group A; Meningococcal Polysaccharide Vaccine Group C; Mumps Virus Vaccine Live; Pertussis Immune Globulin; Pertussis Vaccine; Pertussis Vaccine Adsorbed; Plague Vaccine; Poliovirus Vaccine Inactivated; Poliovirus Vaccine Live Oral; Rabies Immune Globulin; Rabies Vaccine; Rho(D) Immune Globulin; Rubella Virus Vaccine Live; Smallpox Vaccine; Tetanus Antitoxin; Tetanus Immune Globulin; Tetanus Toxoid; Tetanus Toxoid Adsorbed; Typhoid Vaccine; Vaccinia Immune Globulin; Varicella-Zoster Immune Globulin; Yellow Fever vaccine.

Immunomodulator: Dimepranol Acedoben; Imiquimod; Interferon Beta-1b; Lisofylline; Mycophenolate Mofetil; Prozatide Copper Acetate.

Immunoregulator: Azarole; Fanetizole Mesylate; Frentizole; Oxamisole Hydrochloride; Ristianol Phosphate; Thymopentin; Tilomisole.

Immunostimulant: Loxoribine; Teceleukin.

Immunosuppressant: Azathioprine; Azathioprine Sodium; Cyclosporine; Daltroban; Gusperimus Trihydrochloride; Sirolimus; Tacrolimus.

Impotence therapy adjunct: Delequamine Hydrochloride.

Inhibitor: Acarbose; Atorvastatin Calcium; Benserazide; Brocresine; Carbidopa; Clavulanate Potassium; Dazmegrel; Docebenone; Epoprostenol; Epoprostenol Sodium; Epristeride; Finasteride; Flurbiprofen Sodium; Furegrelate Sodium; Lufironil; Miglitol; Orlistat; Pimagedine Hydrochloride; Pirmagrel; Ponalrestat; Ridogrel; Sulbactam Benzathine; Sulbactam Pivoxil; Sulbactam Sodium; Suronacrine Maleate; Tazobactam; Tazobactam Sodium; Ticlopidine Hydrochloride; Tirilazad Mesylate; Tolrestat; Velnacrine Maleate; Zifrosilone; Zileuton.

Keratolytic: Alcloxa; Aldioxa; Dibenzothiophene; Etarotene; Motretinide-I Picotrin Diolamine; Salicylic Acid; Sumarotene; Tazarotene; Tetroquinone; Tretinoin.

LHRH agonist: Deslorelin; Goserelin; Histrelin; Lutrelin Acetate; Nafarelin Acetate.

Liver disorder treatment: Malotilate.

Luteolysin: Fenprostalene.

Memory adjuvant: Dimoxamine Hydrochloride; Ribaminol.

Mental performance enhancer: Aniracetam.

Mood regulator: Fengabine.

Mucolytic: Acetylcysteine; Carbocysteine; Domiodol.

Mucosal Protective agents: Misoprostol (Cytotec).

Mydriatic: Berefrine.

Nasal decongestant: Nemazoline Hydrochloride; Pseudoephedrine Polistirex.

Neuroleptic: Duoperone Fumarate; Risperidone.

Neuromuscular blocking agent: Atracurium Besylate; Cisatracurium Besylate; Doxacurium Chloride; Gallamine Triethiodide; Metocurine Iodide; Mivacurium Chloride; Pancuronium Bromide; Pipecuronium Bromide; Rocuronium Bromide; Succinylcholine Chloride; Tubocurarine Chloride; Vecuronium Bromide.

Neuroprotective: Dizocilpine Maleate.

NMDA antagonist: Selfotel.

Non-hormonal sterol derivative: Pregnenolone Succinate.

Oxytocic: Carboprost; Carboprost Methyl; Carboprost Tromethamine; Dinoprost; Dinoprost Tromethamine; Dinoprostone; Ergonovine Maleate; Meteneprost; Methylergonovine Maleate; Sparteine Sulfate.

Paget's disease agents: Tiludronate Disodium.

Progestin: Algestone Acetophenide; Amadinone Acetate; Anagestone Acetate; Chlormadinone Acetate; Cingestol; Clogestone Acetate; Clomegestone Acetate; Desogestrel; Dimethisterone; Dydrogesterone; Ethynerone; Ethynodiol Diacetate; Etonogestrel; Fluorogestone Acetate; Gestaclone; Gestodene; Gestonorone Caproate; Gestrinone; Haloprogesterone; Hydroxyprogesterone Caproate; Lynestrenol; Medrogestone; Medroxyprogesterone Acetate; Methynodiol Diacetate; Norethindrone Acetate; Norgestimate; Norgestomet; Oxogestone Phenpropionate; Quingestanol Acetate; Quingestrone; Tigestol.

Prostaglandin: Cloprostenol Sodium; Fluprostenol Sodium; Gemeprost; Prostalene; Sulprostone.

Prostate growth inhibitor: Pentomone.

Prothyrotropin: Protirelin.

Psychotropic: Minaprine.

Radioactive agent: Fibrinogen I 125; Fludeoxyglucose F 18; Fluorodopa F 18; Insulin I 125; Insulin I 131; Iobenguane I 123; lodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; lothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; Triolein I 131.

Regulator: Calcifediol; Calcitonin; Calcitriol; Clodronic Acid; Dihydrotachysterol; Etidronic Acid; Oxidronic Acid; Piridronate Sodium; Risedronate Sodium; Secalciferol.

Relaxant: Adiphenine Hydrochloride; Alcuronium Chloride; Aminophylline; Azumolene Sodium; Baclofen; Benzoctamine Hydrochloride; Carisoprodol; Chlorphenesin Carbamate; Chlorzoxazone; Cinflumide; Cinnamedrine; Clodanolene; Cyclobenzaprine Hydrochloride; Dantrolene; Dantrolene Sodium; Fenalamide; Fenyripol Hydrochloride; Fetoxylate Hydrochloride; Flavoxate Hydrochloride; Fletazepam; Flumetramide; Hexafluorenium Bromide; Isomylamine Hydrochloride; Lorbamate; Mebeverine Hydrochloride; Mesuprine Hydrochloride; Metaxalone; Methixene Hydrochloride; Methocarbamol; Nafomine Malate; Nelezaprine Maleate; Papaverine Hydrochloride; Pipoxolan Hydrochloride; Quinctolate; Ritodrine; Ritodrine Hydrochloride; Rolodine; Theophylline Sodium Glycinate; Thiphenamil Hydrochloride; Xilobam.

Repartitioning agent: Cimaterol.

Scabicide: Amitraz; Crotamiton.

Sclerosing agent: Ethanolamine Oleate; Morrhuate Sodium; Tribenoside.

Sedative: Propiomazine.

Sedative-hypnotic: Allobarbital; Alonimid; Alprazolam; Amobarbital Sodium; Bentazepam; Brotizolam; Butabarbital; Butabarbital Sodium; Butalbital; Capuride; Carbocloral; Chloral Betaine; Chloral Hvdrate; Chlordiazepoxide Hydrochloride; Cloperidone Hydrochloride; Clorethate; Cyprazepam; Dexclamol Hydrochloride; Diazepam; Dichloralphenazone; Estazolam Ethchlorvynol; Etomidate; Fenobam; Flunitrazepam; Fosazepam; Glutethimide; Halazepam; Lon-netazepam; Mecloqualone; Meprobamate; Methaqualone; Midaflur; Paraldehyde; Pentobarbital; Pentobarbital Sodium; Perlapine; Prazepam; Quazepam; Reclazepam; Roletamide; Secobarbital; Secobarbital Sodium; Suproclone; Tracazolate; Trepipam Maleate; Triazolam; Tricetamide; Triclofos Sodium; Trimetozine; Uldazepam; Zaleplon; Zolazepam Hydrochloride; Zolpidem Tartrate.

Selective adenosine Al antagonist: Apaxifylline.

Serotonin antagonist: Altanserin Tartrate; Amesergide; Ketanserin; Ritanserin.

Serotonin inhibitor: Cinanserin Hydrochloride; Fenclonine; Fonazine Mesylate; Xylamidine Tosylate.

Serotonin receptor antagonist: Tropanserin Hydrochloride.

Steroid: Dexamethasone Acefurate; Mometasone Furoate.

Stimulant: Amfonelic Acid; Amphetamine Sulfate; Ampyzine Sulfate; Arbutamine Hydrochloride; Azabon; Caffeine; Ceruletide; Ceruletide Diethylamine; Dazopride Fumarate; Dextroamphetamine; Dextroamphetamine Sulfate; Difluanine Hydrochloride; Dimefline Hydrochloride; Doxapram Hydrochloride; Ethamivan; Etryptamine Acetate; Fenethylline Hydrochloride; Flubanilate Hydrochloride; Fluorothyl; Histamine Phosphate; Indriline Hydrochloride; Mefexamide; Methamphetamine Hydrochloride; Methylphenidate Hydrochloride; Pemoline; Pyrovalerone Hydrochloride; Xamoterol; Xamoterol Fumarate.

Suppressant: Amflutizole; Colchicine; Tazofelone.

Symptomatic multiple sclerosis: Fampridine.

Synergist: Proadifen Hydrochloride.

Thyroid hormone: Levothyroxine Sodium; Liothyronine Sodium; Liotrix.

Thyroid inhibitor: Methimazole; Propylthiouracil.

Thyromimetic: Thyromedan Hydrochloride.

Tranquilizer: Bromazepam; Buspirone Hydrochloride; Chlordiazepoxide; Clazolam; Clobazam; Clorazepate Dipotassium; Clorazepate Monopotassium; Demoxepam; Dexmedetomidine; Enciprazine Hydrochloride; Gepirone Hydrochloride; Hydroxyphenamate; Hydroxyzine Hydrochloride; Hydroxyzine Pamoate; Ketazolam; Lorazepam; Lorzafone; Loxapine; Loxapine Succinate; Medazepam Hydrochloride; Nabilone; Nisobamate; Oxazepam; Pentabamate; Pirenperone; Ripazepam; Rolipram; Sulazepam; Taciamine Hydrochloride; Temazepam; Triflubazam; Tybamate; Valnoctamide.

Unstable angina agents: Tirofiban Hydrochloride.

Uricosuric: Benzbromarone; Irtemazole; Probenecid; Sulfinpyrazone.

Vasoconstrictor: Angiotensin Amide; Felypressin; Methysergide; Methysergide Maleate.

Vasodilator: Alprostadil; Azaclorzine Hydrochloride; Bamethan Sulfate; Bepridil Hydrochloride; Buterizine; Cetiedil Citrate; Chromonar Hydrochloride; Clonitrate; Dipyridamole; proprenilamine; Erythrityl Tetranitrate; Felodipine; Flunarizine Hydrochloride; Fostedil; Hexobendine; Inositol Niacinate; Iproxamine Hydrochloride; Isosorbide Dinitrate; Isosorbide Mononitrate; Isoxsuprine Hydrochloride; Lidoflazine; Mefenidil; Mefenidil Fumarate; Mibefradil Dihydrochloride; Mioflazine Hydrochloride; Mixidine; Nafronyl Oxalate; Nicardipine Hydrochloride; Nicergoline; Nicorandil; Nicotinyl Alcohol; Nimodipine; Nisoldipine; Oxfenicine; Oxprenolol Hydrochloride; Pentaerythritol Tetranitrate; Pentoxifylline; Pentrinitrol; Perhexyline Maleate; Pindolol; Pirsidomine; Prenylamine; Propatyl Nitrate; Suloctidil; Terodiline Hydrochloride; Tipropidil Hydrochloride; Tolazoline Hydrochloride; Xanthinol Niacinate.

Vulnerary: Allantoin.

Wound healing agent: Ersofermin.

Xanthine oxidase inhibitor: Allopurinol; Oxypurinol.

Other pharmaceuticals include: 16-Alpha Fluoroestradiol; 16Alpha-Gitoxin; 16-Eplestriol; 17Alpha Estradiol; 17Beta Estradiol; 1Alpha-Hydroxyvitamin D2; 1-Decpyrrolidinone; 1-Dodecpyrrolidinone; 22-Oxacalcitriol; 2CVV; 2′-Nor-cGMP; 3-Isobutyl GABA; 6-FUDCA; 7-Methoxytacrine; Abacavir Sulfate; Abanoquil; Abecarnil; Acadesine; Acamprosate; Acebutolol Hydrochloride; Aceclofenac; Acetomepregenol; Acetrizoate Sodium; Acetylcysteine, N-; Acetyldigitoxin; Acetyl-L-carnitine; Acetylmethadol; Acipimox; Acitemate; Aclatonium; Aconiazide; Acrivastinet; Adafenoxate; Adatanserin; Adefovir Dipivoxil; Adelmidrol; Ademetionine; Adiposin; Adrafinil; Alacepril; Aladapcin; Alaptide; Alatrofloxacin Mesylate; Albolabrin; Albumin Chromated Cr-51 Serum; Albumin Human; Albumin Iodinated I-125 Serum; Albumin Iodinated I-131 Serum; Aldecalmycin; Alendronic Acid; Alentemol; Alfacalcidol; Alfuzosin; Alglucerase; Alinastine; Alitretinoin; Alkavervir; Allopurinol Sodium; Almotriptan Malate; Alosetron; Alpha Idosone; Alpha-Tocopherol; Alpha-Tocopherol Acetate; Alseroxylon; Altromycin B; Amantadine-HCl; Ambenonium Chloride; Amelometasone; Amezinium Metilsulfate; Amfebutamone; Amifloxacin; Aminolevulinic Acid Hydrochloride; Aminosalicylic Acid Resin Complex; Amiodarone Hydrochloride; Amisulpride; Amlodipine; Ammonium Lactate; Amphetamine Adipate; Amphetamine Aspartate; Amphetamine Resin Complex; Ampiroxicam; Amprenavir; Amylin; Amythiamicin; Ananain; Anaritide; Anileridine Phosphate; Anisindione; Anordrin; Apadoline; Apafant; Apraclonidine; Aprepitant; Aprosulate Sodium; Aprotinin Bovine; Aptiganel; Aranidipine; Arbekacin; Arbidol; Arbutamine; Arecatannin B1; Argatroban; Aripiprazol; Aripiprazole; Arotinolol; Articaine Hydrochloride; Ascorbic Acid; Asimadoline; Aspalatone; Asperfuran; Aspoxicillin; Atazanavir Sulfate; Atenolol, S-; Atevirdine; Atomoxetine Hydrochloride; Atpenin B; Atrinositol; Aureobasidin A; Avobenzone; Azadirachtine; Azelaic Acid; Azelastine; Azelnidipine; Azimilide; Azithromycin Dihydrate; Aztreonwn; Baccatin III; Bacoside A; Bacoside B; Bactobolamine; Balazipone; Balhimycin; Balofloxacin; Balsalazide; Bambuterol; Baohuoside 1; Barnidipine; Batebulast; Beauvericin; Becaplermin; Becliconazole; Beclomethasone Dipropionate Monohydrate; Befloxatone; Bellenamine; Benflumetol; Benidipine; Bentoquatam; Benzisoxazole; Benzoidazoxan; Benzoyl Peroxide; Benzphetamine Hydrochloride; Benzquinamide Hydrochloride; Benztropine; Benzyl Benzoate; Benzyl Penicilloyl-Polylysine; Bepridil; Beractant; Beraprost; Berlafenone; Bertosamil; Besipirdine; Beta-Carotene; Betaine, Anhydrous; Betamipron; Betaxolol; Betazole Hydrochloride; Bevantolol; Bexarotene; Bifemelane; Bimakalim; Bimatoprost; Bimithil; Binospirone; Biotin; Bioxalomycin Alpha2; Biriperone; Bisaramil; Bisaziridinylspermine; Bis-Benzimidazole A; Bis-Benzimidazole B; Bismuth Subsalicylate; Bistramide D; Bistramide K; Boldine; Bopindolol; Bortezomib; Brefeldin; Brimonidine; Brinzolamide; Bromfenac; Bucindolol; Budipine; Bunazosin; Butenafine; Butenafine Hydrochloride; Butixocort Propionate; Cabergoline; Caffeine Citrate; Calanolide A; Calcitonin Human; Calcitonin, Salmon; Calcium; Calcium Acetate; Calcium Gluceptate; Calcium Metrizoate; Calfactant; Camonagrel; Candesartan; Candesartan Cilexetil; Candoxatrilat; Capromab; Capsaicin; Carbamazepine; Carbazomycin C; Carbetocin; Carbidopa/Levodopa; Carbovir; Carboxymethylated Beta-1,3-Glucan; Carperitide; Carteolol; Carumonam; Carvotroline; Caspofungin Acetate; Cebaracetam; Cefadroxil/Cefadroxil Hemihydrate; Cefcapene Pivoxil; Cefdaloxime Pentexil Tosilate; Cefditoren Pivoxil; Cefepime Hydrochloride (Arginine Formulation); Cefetamet; Cefetamet Pivoxil; Cefffietazole; Cefluprenam; Cefininox; Cefodizime; Cefoselis; Cefotiam; Cefotiam Hexetil; Cefozopran; Cefpirome; Cefsulodin; Ceftazidime (Arginine Formulation); Ceftazidime Sodium; Cefteram; Ceftibuten Dihydrate; Ceftriaxone; Celastrol; Celecoxib; Celikalim; Celiprolol; Cellulose Sodium Phosphate; Cepacidine A; Cericlamine; Cerivastatin; Cerivastatin Sodium; Certoparin Sodium; Cetiedil; Cetirizine; Cetyl Alcohol; Cevimeline Hydrochloride; Chlormerodrin, Hg-197; Chlormezanone; Chloroorienticin A; Chloroorienticin B; Cholecalciferol; Cholestyramine; Choriogonadotropin Alfa; Chromic Phosphate, P-32; Chymopapain; Chymotrypsin; Cibenzoline; Ciclesonide; Cicloprolol; Cilansetron; Cilnidipine; Cilobradine; Cilostazol; Cimetropiurn Bromide; Cinitapride; Cinolazepam; Ciprostene; Cisapride Monohydrate; Cisatracurium, Besilate; Cistinexine; Citalopram; Citalopram Hydrobromide; Citicoline; Citreamicin Alpha; Clausenamide; Clidinium Bromide; Clinafloxacin; Clomethiazole; Clopidogrel; Clopidogrel Bisulfate; Cobalt Chloride, Co-57; Cobalt Chloride, Co-60; Colesevelam Hydrochloride; Colestimide; Colfosceril Palmitate; Complestatin; Contignasterol; Contortrostatin; Corticotropin-Zinc Hydroxide; Cosalane; Costatolide; Cotinine; Cournermycin AI; Cryptenamine Acetates; Cryptenamine Tannates; Cucumariosid; Curdlan Sulfate; Curiosin; Cyanocobalamin; Cyanocobalamin, Co-57; Cyanocobalamin, Co-58; Cyanocobalamin, Co-60; Cyclazosin; Cyclic HPMPC; Cyclobenzaprine; Cyclobut A; Cyclobut G; Cyclocapron; Cyclosin; Cyclothialidine; Cyclothiazomycin; Cycrimine Hydrochloride; Cyproterone; Cysteamine Bitartrate; Cytochalasin B; Dactimicin; Daidzein; Daidzin; Danaparoid; Daphnodorin A; Dapiprazole; Dapitant; Darifenacin; Darlucin A; Darsidomine; Daunorubicin Citrate; DdUTP; Decamethonium Bromide; Deferiprone; Deferoxamine Mesylate; Dehydrodidemnin B; Delapril; Delequamine; Delfaprazine; Delmopinol; Delphinidin; Deoxypyridinoline; Deprodone; Depsidomycinderamciclane; Dermatan Sulfate; Deserpidine; Desirudin; Desloratadine; Desmopressin; Desoxoamiodarone; Desoxyribonuclease; Detajrniurn Bitartrate; Dexketoprofen; Dexioxiglumide; Dexmethylphenidate Hydrochloride; Dexrazoxane Hydrochloride; Dexsotalol; Dextrin 2-Sulphate; Dextroamphetamine Adipate; Dextroamphetamine Resin Complex; Dextroamphetamine Saccharate; Dextrose; Diclofenac Digolil; Dicranin; Dienogest; Diethylhomospennine; Diethylnorspermine; Difenoxin Hydrochloride; Dihydrexidine; Diltiazeim; Dimethyl Prostaglandin Al; Dimethylhomospermine; Dimiracetam; Dimyristoyl Lecithin; Diphemanil Methylsulfate; Diphencyprone; Diphenylpyraline Hydrochloride; Diprafenone; Dipropylnorspermine; Discodermolide; Divalproex; Docarpamine; Docosanol, 1-; Dolasetron Mesylate Monohydrate; Domitroban; Donepezil Hydrochloride; Dorzolamide; Dosmalfate; Dotarizine; Doxazosin; Doxercalciferol; Draculin; Drosperidone; Drospirenone; Drotaverine Acephyllinate; Droxicam; Dutasteride; Ebiratide; Ebrotidine; Ecabapide; Ecabet; Ecdisteron; Echicetin; Echistatin; Ecteinascidin 722; Ecteinascidin 729; Ecteinascidin 743; Edaravone; Edetate Calcium Disodium; Edetate Disodium; Edobacomab; Edrecolomab; Efavirenz; Efegatran; Efonidipine; Egualen; Elcatonin; Eletriptan; Eletriptan Hydrobromide; Elgodipine; Eliprodil; Eltenac; Emakalim; Emedastine; Emedastine Difumarate; Emiglitate; Emoctakin; Emtricitabine; Enalapril; Enazadrem; Enfuvirtide; Englitazone; Entacapone; Enterostatin; Eplerenone; Epoxymexrenone; Eptastigmine; Eptifibatide; Erdosteine; Ergocalciferol; Ersentilide; Ertapenem Sodium; Erythritol; Escitalopram Oxalate; Esomeprazole Magnesium; Estazolam; Estradiol Acetate; Esuprone; Etanterol; Ethacizin; Ethchlorvynol; Ethinamate; Ethinylestradiol; Ethoxzolamide; Etidocaine Hydrochloride; Etizolam; Etrabamine; Eveminomicin; Examorelin; Ezetimibe; Faerieftmgin; Fantofarone; Farnciclovir; Faropenem; Fasidotril; Fasudil; Fedotozine; Felbamate; Fenofibrate; Fenoldopam; Fenspiride; Fentanyl; Fenticonazole; Fepradinol; Ferpifosate Sodium; Ferristene; Ferrixan; Ferrous Citrate, Fe-59; Fexofenadine Hydrochloride; Fibrinogen, I-125; Fibrinolysin; Flecamide; Flerobuterol; Flesinoxan; Flezelastine; Flobufen; Flomoxef; Florfenicol; Florifenine; Flornastat; Flosatidil; Fludeoxyglucose, F-18; Flumecinol; Flunarizine; Fluocalcitriol; Fluoxetine, R-; Fluoxetine, S-; Fluparoxan; Flupirtine; Flurbiprofen Axetil; Flurithromycin; Flutamide; Flutrimazole; Fluvastatin; Fluvoxamine; Folic Acid; Follitropin Alfa; Follitropin Alfa/Beta; Fomivirsen Sodium; Fondaparinux Sodium; Forasartan; Formoterol; Formoterol Fumarate; Formoterol, R,R-; Fosinopril; Fosphenyloin; Frovatriptan Succinate; Fulvestrant; Furosernide; Gadobenic Acid; Gadobutrol; Gadodiamide-EOB-DTPA; Gadopentetate Dimeglumine; Gadoteric Acid; Galantamine; Galantamine Hydrobromide; Galdansetron; Gallopamil; Gamolenic Acid; Gatifloxacin; Gefitinib; Gemifloxacin Mesylate; Gemtuzumab Ozogamicin; Gepirone; Girisopam; Glaspimod; Glatiramer Acetate; Glaucocalyxin A; Glucagon Hydrochloride; Glucagon Hydrochloride Recombinant; Glucagon Recombinant; Gluconolactone; Glutapyrone; Glutathione Disulfide; Glycopine; Glycopril; Goserelin Acetate; Grepafloxacin; Grepafloxacin Hydrochloride; Guaifenesin; Guanidine Hydrochloride; Halichondrin B; Halofantrine; Halomon; Haloperidol Lactate; Halopredone; Hatomarubigin C; Hatomambigin D; Hatomamicin; Hatornarubigin A; Hatornarubigin B; Heparin Calcium; Heparin Sodium; Hexocyclium Methylsulfate; Hexylcaine Hydrochloride; Histrelin Acetate; Hyaluronidase; Hydrocortamate Hydrochloride; Hydrocortisone Cypionate; Hydrocortisone Probutate; Hydroquinone; Hydroxocobalamin; Hydroxypropyl Cellulose; Hydroxystilbamidine Isethionate; Ibandronate Sodium; Ibogaine; Ibudilast; Ibuprofen Potassium; Icodextrin; Illimaquinone; Iloprost; Imatinib Mesylate; Imidapril; Imidazenil; Imiglucerase; Imipramine Pamoate; Inaminone Lactate; Indapamide; Indinavir; Indinavir Sulfate; Indium In-111 Oxyquinoline; Indium In-111 Pentetate Disodium; Indium In-111 Pentetreotide Kit; Indometacin; Indometacin Farnesil; Indomethacin Sodium; Inocoterone; Inogatran; Inolimomab; Insulin Aspart; Insulin Aspart Protamine; Insulin Glargine; Insulin Lispro Protamine; Interferon Alfa; Interferon Alfa-NI; Interferon Beta; Interferon Beta-lal; Interferon Gamma-I A; Interferon Gamma-I B; Interferon Omega; Interferon, Consensus; Interleukin-3; Interleukin-1; Interleukin-I Beta; Interleukin-10; Interleukin-1; Interleukin-12; Interleukin-15; Interleukin-2; Interleukin-4; Interleukin-5; Interleukin-7; Interleukin-8; InterleukinI Alpha; Intrinsic Factor; Inulin; Invert Sugar; lobenguane Sulfate I 131; lobitridol; Iodamide Meglumine; lodipamide Sodium; Iodoamiloride; Iodohippurate Sodium, I-123; Iodohippurate Sodium, I-131; Iofetamine Hydrochloride I-123; Iofratol; Iopromide; lopyrol; Iomeprol; lothalamate Sodium, I-125; Iotriside; Ioxaglate Sodium; Ipazilide; Ipenoxazone; Ipidacrine; Ipomeanol, 4; Ipriflavone; Ipsapirone; Irbesartan; Irloxacin; Iron Dextran; Iron Sucrose; Irtemazole; Isalsteine; Isbogrel; Isepamicin; Isofloxythepin; Isopropyl Unoprostone; Itameline; Itopride; Ketoprofen, R-; Ketoprofen, S-; Ketorolac; Lactitol; Lactivicin; Lactulose; Laennec; Lafutidine; Lanoconazole; Lanperisone; Larnifiban; Larnotrigine; Latanoprost; Lateritin; Laurocaprarn; Leflunomide; Lemefloxacin; Leminoprazole; Lenercept; Lepirudin; Leptin; Lercanidipine; Lerisetron; Lemildipine; Lesopitron; Letrazuril; Leucomyzin; Levalbuterol Hydrochloride; Levallorphan Tartrate; Levamisole Hydrochloride; Levetiracetam; Levobetaxolol; Levobunolol; Levobupivacaine; Levobupivacaine Hydrochloride; Levocabastine; Levocarnitine; Levodropropizine; Levofloxacin; Levopropoxyphene Napsylate, Anhydrous; Levormeloxifene; Levornoprolol; Levosimendan; Levosulpiride; Lindane; Linezolid; Linotroban; Linsidornine; Lintitript; Lintopride; Lipase; Lirexapride; Lithium Carbonate; Lithium Citrate; Lodoxamide; Lomerizine; Lonazolac; Lopinavir; Lorglumide; Losartan; Losigamone; Loteprednol; Loviride; Loxapine Hydrochloride; LpdR; Lubeluzole; Lutetium; Luzindole; Lydicamycin; Lysostaphin; Magainin 2 Amide; Magnesium Acetate; Magnesium Acetate Tetrahydrate; Magnolol; Malathion; Mallotochromene; Mallotojaponin; Mangafodipir; Mangafodipir Trisodium; Manidipine; Maniwamycin A; Mannitol; Manumycin E; Manumycin F; Mapinastine; Martek 8708; Martek 92211; Massetolide; Meglumine Metrizoate; Meloxicam; Melphalan Hydrochloride; Menadiol Sodium Diphosphate; Menadione; Meprednisone; Mequinol; Mersalyl Sodium; Mesna; Metformin Hydrochloride; Methantheline Bromide; Metharbital; Methoxamine Hydrochloride; Methoxatone; Methoxsalen; Methscopolamine Bromide; Methyclothiazide; Methyldopa; Methylhistamine, R-alpha; Methylinosine Monophosphate; Methylprednisolone Aceponate; Methyprylon; Metipamide; Metipranolol Hydrochloride; Metolazone; Metoprolol Fumarate; Metoprolol, S-; Metoprotol Tartrate; Metrifonate; Metrizoate Magnesium; Metrizoic Acid; Mezlocillin Sodium Monohydrate; Michellamine B; Microcolin A; Midodrine; Miglustat; Milacemide; Milarneline; Mildronate; Milnacipran; Milrinone Lactate; Miokarnycin; Mipragoside; Mirfentanil; Mivazerol; Mixanpril; Mizolastine; Mizoribine; Moexipril; Moexipril Hydrochloride; Mofezolac; Mometasone; Mometasone Furoate Monohydrate; Monobenzone; Montirelin; Moracizine; Moricizine Hydrochloride; Mosapramine; Mosapride; Motilide; Moxifloxacin Hydrochloride; Moxiraprine; Moxonidine; Mupirocin; Mupirocin Calcium; Mycophenolate Mofetil Hydrochloride; Nadifloxacin; Nadroparin Calcium; Nafadotride; Nafamostat; Naftopidil; Naglivan; Nalmefene Hydrochloride; Naltrexone Hydrochloride; Napadisilate; Napsagatran; Naratriptan; Nasaruplase; Nateglinide; Nateplasel; Nelfinavir Mesylate; Nesiritide; Niacinamide; Nicotine; Nicotine Polacrilex; Niperotidine; Niravoline; Nisin; Nitazoxanide; Nitecapone; Nitisinone; Nitrendipine, S-; Nitrofurantoin Monohydrate; Nitrofurantoin Sodium; Nitrofurantoin, Macrocrystalline; Nitrofurazone; Nitroglycerin; Nonoxynol-9; Norelgestromin; Octyl Methoxycinnamate; Olmesartan Medoxomil; Olopatadine; Olopatadine Hydrochloride; Olprinone; Olsalazine; Omeprazole Magnesium; Ondansetron, R-; Oral Hypoglyceremics; Orphenadrine Hydrochloride; Oseltamivir Phosphate; Otenzepad; Oxamisole; Oxaprozin Potassium; Oxcarbazepine; Oxiconazole; Oxiracetam; Oxodipine; Oxybenzone; Oxybutynin; Oxyphencyclimine Hydrochloride; Oxyphenonium Bromide; Ozagrel; Palauamine; Palinavir; Palonosetron Hydrochloride; Pamaparin Sodium; Panamesine; Pancrelipase; Panipenem; Panipenum; Pannorin; Panornifene; Pantethine; Pantoprazole Sodium; Pantothenic Acid; Paramethadione; Paricalcitol; Pamaqueside; Pamicogrel; Paroxetine Hydrochloride; Paroxetine Mesylate; Parthenolide; Pazufloxacin; Pegademase Bovine; Pegvisomant; Pemirolast; Pemirolast Potassium; Penciclovir Sodium; Penicillamine; Pentafuside; Pentagastrin; Pentamidine; Pentamidine Isethionate; Pentetate Calcium Trisodium Yb-169; Pentigetide; Pentolinium Tartrate; Pentosan; Perflexane; Perfluoropolymethylisopropyl Ether; Perflutren; Pergolide; Pergolide Mesylate; Perindoprilat; Pemedolac; Perospirone; Phenaridine; Phenindione; Pheniramine Maleate; Phenmetrazine Hydrochloride; Phenotoxifvline; Phenserine; Phensuccinal; Phentermine Resin Complex; Phentolamine Mesilate; Phenylalanyl Ketoconazole; Phenylephrine Bitartrate; Phenyloin Sodium, Extended; Phenyloin Sodium, Prompt; Phosphoric Acid; Phytonadione; Picenadol; Picroliv; Picumeterol; Pidotimod; Pilsicamide; Pimagedine; Pimecrolimus; Pimilprost; Pinocebrin; Pioglitazone; Piperonyl Butoxide; Pirlindole; Pirmenol; Pirodomast; Polyestradiol Phosphate; Polyethylene Glycol 3350; Polytetrafluoroethylene; Poractant Alfa; Potassium Chloride; Pramipexole Dihydrochloride; Praziquantel; Prazosin; Prilocalne; Procaine Merethoxylline; Proguanil Hydrochloride; Propagermanium; Propentofylline; Propiolactone; Propiomazine Hydrochloride; Propionylcamitine, L-; Propiram; Propiram+Paracetamol; Propiverine; Prostratin; Protegrin; Protein Hydrolysate; Protokylol Hydrochloride; Protosufloxacin; Prulifloxacin; Pyrethrins; Pyridoxine; Pyridoxine Hydrochloride; Quazepam; Quetiapine; Quetiapine Fumarate; Quiflapon; Quinagolide; Quinapril; Quinethazone; Quinidine Polygalacturonate; Raloxifene; Ramatroban; Ranelic Acid; Ranolazine; Rapacuronium Bromide; Recainarn; Regavirumab; Repaglinide; Rescinnamine; Resinferatoxin; Reticulon; Reviparin Sodium; Revizinone; Riboflavin; Riboflavin Phosphate Sodium; Ricasetron; Rilopirox; Rimantadine; Rimexolone; Rimoprogin; Riodipine; Ripisartan; Risedronic Acid; Rispenzepine; Ritipenem Acoxil; Ritipenern; Ritonavir; Rivastigmine Tartrate; Rizatriptan Benzoate; Rnibefradil; Rnivacurium Chloride; Rofecoxib; Rokitamycin; Ropinirole; Ropivacaine; Ropivacaine Hydrochloride Monohydrate; Roquinimex; Rose Bengal Sodium, I-131; Rosiglitazone Maleate; Roxatidine; Roxindole; Rubidium Chloride Rb-82; Rufloxacin; Rupatidine; Ruzadolane; Sacrosidase; Safflower Oil; Safironil; Salbutamol, R-; Salnacedin, R-; Samarium Sm 153 Lexidronam Pentasodium; Sanfetrinem; Saprisartan; Sapropterin; Saquinavir; Sarcophytol A Sargramostim; Sameridine; Sampatrilat; Sarpogrelate; Saruplase; Saterinone; Satigrel; Satumomab Pendetide; Scopolamine; Secretin; Selenomethionine, Se-75; Sematilide; Sermorelin; Semotiadil; Sertaconazole; Sertraline; Sertraline-HCl; Setiptiline; Sevelamer Hydrochloride; Sevirurnab; Sezolamide; Sildenafil Citrate; Silipide; Silteplase; Silver Sulfadiazine; Simendan; Simethicone; Simethicone-Cellulose; Sinitrodil; Sinnabidol; Sipatrigine; Sirnvastatin; Somatomedin C; Somatropin Recombinant; Sorbitol; Somatomedin B; Sornatrem; Sornatropin; Sotalol; Staurosporine; Stepronin; Stobadine; Strontium Chloride, Sr-89; Succibun; Sulfanilamide; Sulfaphenazole; Sulfapyridine; Sulfoxamine; Sulfoxone Sodium; Sulfur; Sultamicillin; Sultopride; Sumatriptan; Sutilains; Symakalim; Talbutal; Tandospirone; Tannic Acid; Tapgen; Taprostene; Tartaric Acid; Tazanolast; Tegaserod Maleate; Telenzepine; Telmesteine; Telmisartan; Temocapril; Tenofovir Disoproxil Fumarate; Tenosal; Tepirindole; Terazosin; Terbinafine Hydrochloride; Terflavoxate; Terguride; Terlipressin; Terodiline; Tertatolol; Testosterone Buciclate; Thallous Chloride, TI-201; Thiamine; Thiamine Hydrochloride; Thiofedrine; Thiomarinol; Thioperamide; Thiosemicarbazone; Thonzonium Bromide; Thyroglobulin; Thyrotropin; Thyrotropin Alfa; Tiagabine; Tiagabine Hydrochloride; Tianeptine; Tiapafant; Ticlopidine; Tienoxolol; Tilisolol; Tilnoprofen Arbamel; Tiludronic Acid; Tiopronin; Tiotropium Bromide; Tirandalydigin; Tirilazad; Tirofiban; Tiropramide; Tocopherol Acetate; Tolterodine Tartrate; Torasemide; Trafennin; Trandolapril; Tranylcypromine Sulfate; Travoprost; Traxanox; Trazodone-HCl; Treprostinil Sodium; Tretinoin Tocoferil; Triarntevene; Tricaprilin; Trichohyalin; Trichosanthin, Alpha; Triclosan; Tridihexethyl Chloride; Trientine; Trientine Hydrochloride; Triflavin; Trimegestone; Trimethoprim Hydrochloride; Trioxsalen; Triptorelin Pamoate; Trolamine Polypeptide Oleate Condensate; Trombodipine; Trometarnol; Tromethamine; Tropine Ester; Trospectomycin; Trovafloxacin; Trovafloxacin Mesylate; Trovirdine; Tucaresol; Tulobuterol; Tylogenin; Tyloxapol; Undecoylium Chloride; Undecoylium Chloride Iodine Complex; Unoprostone Isopropyl; Urapidil; Urea, C-13; Urea, C-14; Uridine Triphosphate; Valaciclovir; Valdecoxib; Valganciclovir Hydrochloride; Valproate Magnesium; Valproate Semisodium; Valrubicin; Valsartan; Vamicamide; Vanadeine; Vaninolol; Vasopressin Tannate; Venlafaxine; Verapamil, (S); Veratrum Viride; Veroxan; Vexibinol; Vinbumine Citrate; Vinbumine Resinate; Vinconate; Vinpocetine; Vinpocetine Citrate; Vintoperol; Viomycin Sulfate; Vitamin A; Vitamin A Palmitate; Vitamin E; Vitamin K; Voriconazole; Voxergolide; Warfarin Potassium; Xemilofiban; Ximoprofen; Yangambin; Zabicipril; Zacopride; Zacopride, R-; Zafirlukast; Zalospirone; Zaltoprofen; Zanamivir; Zanamivir; Zankiren; Zatebradine; Zatosetron; Zenarestat; Zinostatin Stimalamer; Ziprasidone; Ziprasidone Mesylate; Zoledronic Acid; Zolmitriptan; Zolpidern; Zopiclone; Zopiclone, S-; Zopolrestat; Zotepine.

Still other examples of pharmaceuticals are listed in 2000 MedAd News 19:56-60 and The Physicians Desk Reference, 53rd. Edition, pages 792-796, Medical Economics Company (1999), both of which are incorporated herein by reference.

Examples of suitable veterinary pharmaceuticals for used with SCOPE formulations include, but are not limited to, vaccines, antibiotics, growth enhancing components, and dewormers. Other examples of suitable veterinary pharmaceuticals are listed in The Merck Veterinary Manual, 8th Edition, Merck and Co., Inc., Rahway, N.J., 1998; (1997); The Kirk-Othmer Encyclopedia of Chemical Technology, Volume 24 Kirk-Othmer (4th Edition at page 826); and Veterinary Drugs by A. L. Shore and R. J. Magee, American Cyanamid Co. in The Encyclopedia of Chemical Technology 2nd. Edition, Volume 21, all of which are incorporated herein by reference.

“Potency phase map” means a plot of the magnitude of penetration enhancement as a function of two or more compositional variables.

“Sample” or equivalently “formulation” means a component or a mixture of a plurality of components. A sample typically contains at least one active component and at least one inactive component, although this is not a requirement. For example, approximate measurements of penetration enhancement may be made on samples containing a chemical penetration enhancer or a combination of chemical penetration enhancers, usually with a solvent, but without an active component. Samples and formulations can take many forms, which include, without limitation, solids, semisolids, liquids, solutions, emulsions, suspensions, triturates, gels, films, foams, pastes, ointments, adhesives, highly viscoelastic liquids and any of the foregoing having solid particulates dispersed therein.

When performing high throughput experimentation on samples it is preferred that the samples are placed in an array format. Samples in a sample array may each comprise a different composition, or the sample array may contain replicate samples, standards and/or blanks. A sample can be present in any container or holder or in or on any material or surface. Preferably, the samples are located at separate sites. Preferably, where samples are in an array format, samples are contained an array of sample wells, for example, a 24, 36, 48, 96, 384 or 1,536 well plate array. The sample can comprise less than about 100 milligrams of an active component, preferably, less than about 1 milligram, more preferably, less than about 100 micrograms, and even more preferably, less than 100 nanograms. Preferably, the sample has a total volume of about 1-200 μl, more preferably about 5-150 μl, and most preferably about 10-100 μl.

“Skin” means the tissue layer forming the external covering of the body of a human or animal, which is in turn characterized by a number of sub-layers such as the dermis, the epidermis and the stratum corneum.

“Skin care actives” means all compounds or substances now known or later demonstrated to provide benefit when applied to the skin of patients or consumers and all compounds now claimed or in the future claimed to provide benefit when applied to the skin of patients or consumers. Skin care actives may provide benefits, or claimed benefits, in areas such as wrinkle removal or wrinkle reduction, firming of skin, exfoliation of skin, skin lightening, treatment of dandruff, treatment of acne, skin conditioning, development of tans and artificial tans, improvement of skin moisture content, improvement of skin barrier properties, control of sweat, anti-ageing, reduction or avoidance of irritation and reduction or avoidance of inflammation. Skin care actives can be molecules such as protease and/or other enzyme inhibitors, anti-coenzymes, chelating agents, antibodies, antimicrobials, humectants, vitamins, skin protectants and/or skin soothing agents, plant extracts and the like. Examples of skin care actives include but are not limited to vitamin C, vitamin E (alpha tocopherol), retinoids, soy derivatives (e.g. isoflavones), green tea polyphenols, alpha hydroxy acids (e.g. glycolic and lactic acid), beta hydroxy acids (e.g. salicylic acid), poly hydroxy acids, alpha lipoic acid, hemp oil (glycerides), niacinamide, dimethyl aminoethanol, coenzyme Q10, kinetin (plant growth hormone), dimethyl sulfone and botulinum toxin.

“Solvent” means a fluid in which a component such as an active component, carrier, or adhesive will dissolve. Solvents are selected based on the solubility of the component to be dissolved, chemical compatibility, biocompatibility and other factors. Aqueous solvents can be used to make matrices formed of water soluble polymers. Organic solvents will typically be used to dissolve hydrophobic and some hydrophilic components. Preferred organic solvents are volatile or have a relatively low boiling point or can be removed under vacuum and which are acceptable for administration to humans in trace amounts, such as methylene chloride. Other solvents, such as ethyl acetate, ethanol, methanol, dimethyl formamide (DMF), acetone, acetonitrile, tetrahydrofuran (THF), acetic acid, dimethyl sulfoxide (DMSO) and chloroform, and combinations thereof, also may be utilized. Preferred solvents are those rated as class 3 residual solvents by the Food and Drug Administration, as published in the Federal Register vol. 62, number 85, pp. 24301-24309 (May 1997), incorporated herein by reference. Solvents for drugs will typically be distilled water, phosphate buffered saline (“PBS”), Lactated Ringer's or some other pharmaceutically acceptable carrier.

“Synergy value” between two CPEs, A and B, in a formulation is calculated using the following equation:

$$S = \frac{{ER}_{A + B} ( X , Y )}{X \cdot {ER}_{A} ( Y ) + ( 1 - X ) \cdot {ER}_{B} ( Y )} ,$$

where ER_{A+B}(X,Y) is the enhancement ratio obtained with the formulation containing CPEs A and B, Y stands for the combined amount of A and B expressed in wt/vol and X stands for the weight fraction of A computed as the amount of A in formulation (expressed in wt/vol) divided by Y. ER_{A}(Y) and ER_{B}(Y) are measured by preparing formulations whose composition is the same as that containing the CPEs A and B except that CPEs A and B are replaced with either pure component A at a wt/vol of Y or pure component B at a wt/vol of Y. ER_{A}(Y) and ER_{B}(Y) are then the enhancement ratios measured for the formulation in which A, but not B, is present and B, but not A, is present, respectively. Enhancement ratios, and as a consequence synergy values, are a function of time and it is understood that the enhancement ratios in the above equation should be measured at equal times. The term “t-hour synergy value” is understood to mean the synergy value calculated using t-hour enhancement ratios, where t hours may be any period of time over which enhancement ratios may be reasonably measured.

“Test membrane” means a membrane that is suitable for use in a diffusion cell experiment. A test membrane may be natural or synthetic skin or related tissue such as mucosal tissue, preferably stratum corneum or skin tissue, such as hairless mouse skin, porcine skin, guinea pig skin, or human skin. If human cadaver skin is to be used, one known method of preparing the test membrane entails heat stripping by keeping it in water at 60° C. for two minutes followed by the removal of the epidermis, and storage at 4° C. in a humidified chamber; a piece of epidermis is taken out from the humidified chamber prior to the experiments and optionally supported by a porous support such as Nylon mesh (available from Sefar America Inc. (Tetko Inc.) of Depew, N.Y.; www.sefaramerica.com, or Fisher Scientific of Pittsburgh, Pa.; www.fishersci.com) to avoid damage and to mimic the fact that the skin in vivo is supported by mechanically strong dermis. Other types of tissues may also be used, including living tissue explants, any of a number of endothelial or epithelial cell culture barriers, such as those described in Audus, et al., animal tissue (e.g. rodent, bovine or swine) or engineered tissue-equivalents. Audus et al. (1990). Examples of a suitable engineered tissues include DERMAGRAFT®, a human fibroblast-derived dermal substitute (available from Smith & Nephew, Inc. of Largo Fla.; www.dermagraft.com) and those taught in U.S. Pat. No. 5,266,480, which is incorporated herein by reference. A synthetic membrane, such as an elastomeric membrane, may also be used. The nature of the test membrane is membrane is preferably chosen based in the desired application. Screening of formulations for transdermal delivery is preferably conducted using pigskin; whereas to screen formulations for buccal, vaginal, nasal drug delivery and the like, mucosal membrane might be used, and so forth.

“Therapeutically effective amount” means a sufficient amount of a drug to provide the desired therapeutic effect or other desired effect, for example, a prophylactic effect.

“Transdermal drug delivery” or “transdermal drug administration” refers to administration of a drug to the skin surface of an individual so that the drug passes through the skin tissue and into the individual's blood stream. The term “transdermal” is intended to include “transmucosal” drug administration, i.e., administration of a drug to the mucosal (e.g., sublingual, buccal, vaginal, rectal) surface of an individual so that the drug passes through the mucosal tissue and into the individual's blood stream.

“Topical drug delivery” or “topical drug administration” is used in its conventional sense to mean delivery of a topical drug of a pharmacologically active agent to the skin or mucosa, as in, for example, the treatment of various skin disorders. Topical drug administration, in contrast to transdermal administration, is often used to provide a local rather than a systemic effect.

“21-day cumulative irritation test” refers to the 21-day patch test described by Berger and Bowman (1982) entitled “A reappraisal of the 21-day cumulative irritation test in man,” and acceptable variations and modifications thereof.

“21-day cumulative irritation test score” means the score achieved by a formulation on the 630 point scale of the 21-day cumulative irritation test described by Berger and Bowman (1982). The 21-day cumulative irritation test score is a measure of irritation potential and acceptable variations and modifications thereof.

For a rapid assessment of combinations of penetration enhancers, a high throughput experimentation system has been developed. Karande et al. (2002), and International Application Number PCT/US01/26473 entitled “A Combinatorial Method For Rapid Screening Of Drug Delivery Formulations”, published under International Publication Number WO 02/16941 A2. The HTE system provides an efficient method to monitor the depletion of a test substance from a donor well, the migration of the test substance into a test membrane, and/or the migration of the test substance through a test membrane into a receptor well. A test membrane is secured to a donor plate having a plurality of through holes forming donor wells. Formulations are introduced into donor wells and a characteristic of the test substance that remains in the donor well or migrates into the test membrane is evaluated. A receptor plate can be provided that is formed with receptor wells that correspond to the donor wells, the test membrane being secured between the donor plate and the receptor plate. The device can further include electrodes to measure current across the test membrane.

Transdermal and Topical Drug Delivery: Transdermal drug delivery can be used to circumvent first pass metabolism and provide a sustained drug release for a prolonged period of time. Topical drug delivery allows a drug to be applied directly to the surface of area to be treated, which can be useful to localize the treatment and minimize side effects. Evolved to impede the flux of toxins into the body, skin however offers a very low permeability to the movement of foreign molecules across it. The stratum corneum is responsible for this barrier. It possesses a unique hierarchical structure of lipid rich matrix with embedded keratinocytes in the upper strata (15 μm) of skin. Bouwstra (1997). Overcoming this barrier safely and reversibly is a fundamental problem that persists today in the field of transdermal delivery. Although more than two hundred and fifty chemical enhancers including surfactants, azone and related chemicals, fatty acids, fatty alcohols, fatty esters, and organic solvents have been tested to increase transdermal drug transport, only a handful are actually used in practice. Berti et al. (1995). This discrepancy results from the fact that among all the enhancers that have been used, only a few induce a significant (therapeutic) enhancement of drug transport, Walters (1989); Finnin (1999). Furthermore skin irritation and safety issues limit the applications of several enhancers. These limitations are overcome by the invention and the use of special combinations of chemical penetration enhancers.

Most molecules known as potent enhancers in the literature are also potent irritants. Very few molecules that show therapeutically significant enhancements are physiologically compatible. This remains a limiting step in exploiting transdennal delivery as an efficient delivery mode. By combining two or more penetration enhancers, the concentration of each enhancer required to achieve desired enhancements may be lower than that required if any one of the enhancers was used individually. There is limited literature data available on combinations of chemical enhancers. Mollgaard (1993); Funke et al. (2002); Karande et al. (2000).

In screening as contemplated herein a large diverse library of component combinations, for example, is selected from the above categories of enhancers, either randomly or based on knowledge about the mechanism of action of the enhancers. If individual enhancers increase transdermal transport via different mechanisms, their combination can be more effective than either of them alone. Chemical penetration enhancers increase skin permeability by reversibly disrupting or by altering the physiochemical nature of the stratum corneum to reduce its diffusional resistance. Typically a penetration may enhancer increase SC penetrability by any of the following mechanisms, for example (Shah, et al. (1993)):
fluidizing the crystalline structure of SC by incorporating itself in the lipid bilayer;
dissolving skin lipids by forming mixed aggregates with lipid molecules;
acting as a co-solvent for the drug, thereby driving more drug into the solvent phase;
increasing the partition coefficient of drug in skin and thus increasing its distribution in the lipid matrix; and/or
altering polar or non-polar pathways in the multilaminate lipid matrix.

A combination of two molecules chosen from two independent categories above may better than either of them alone. An enhancer A that fluidizes the bilayer and an enhancer B that forms mixed aggregates with the skin lipids may work, for example, in either of the following ways:

component A fluidizes the bilayer, facilitating dissolution of lipid molecules in the bilayer by component B; and/or component B dissolves the bilayer thus facilitating incorporation of component A into the bilayer.

In either case, or for other reasons, the combination of A and B may work better than A or B by itself. An example of this possibility is provided by the work of Karande et al., where high throughput screening experiments revealed that mixtures of sodium lauryl sulfate and dodecyl pyridinium chloride are significantly more effective in enhancing transdermal transport compared to each of them alone. Karande et al. (2000). It might be thought that the enhancement of penetration by formulations containing A and B would vary in a gradual fashion as the concentration of A and B are varied and that the irritation potential of highly penetrating formulations will be tend to be higher than the irritation potential of less penetrating formulations. Surprisingly, however it has been discovered that a combination of penetration enhancers can yield sharp maxima about which the penetration rates varies rapidly with the concentration of constituent CPEs. A further surprise is that compositions in the vicinity of these maxima in composition space showing exceptional penetration enhancement can also on occasion have exceptionally low irritation potential.

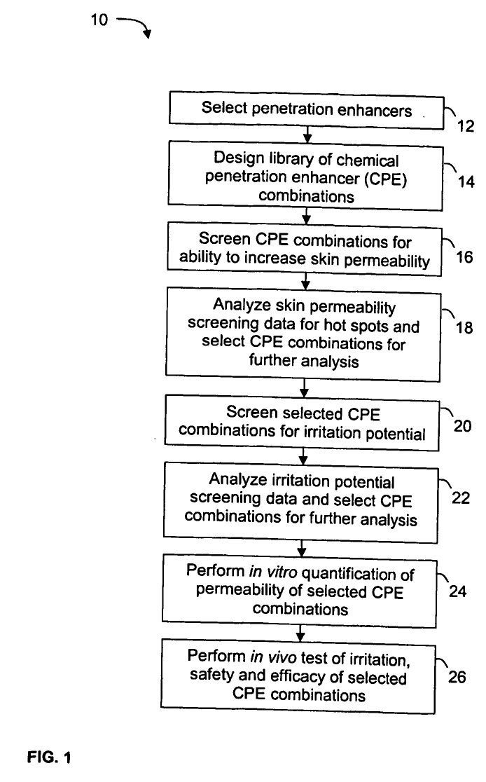

FIG. 1 is a flow chart 10 showing in general terms, a sequence of steps that may be applied to identify SCOPE compositions according to one embodiment of the invention. The first step, 12, is to select individual penetration enhancers. Then, if desired, a library of CPE combinations is designed, at 14. One or more combinations or a library may be screened, at 16, for the ability of the CPE combinations to increase skin permeability. The screening data may be analyzed, at 18, for hot spots and selected CPE combinations may be selected for further analysis and measurement of irritation potential, at 20. The irritation potential data may be analyzed, at 22, and a refined list of CPE combinations developed for further analysis. The selected CPE combinations may be combined with a selected drug and in vitro quantification performed, at 24. Finally, candidate combinations may be selected from, for example, the in vitro quantification data and in vivo tests are performed, at 26, for irritation, safety and efficacy.

It can be seen that the sequence of steps provided in FIG. 1 provides a procedure whereby a pool of candidate formulations may be progressively narrowed to smaller subsets of the initial pool. The formulations remaining under investigation may tend to become, on average, better suited to the task of delivering active components in topical or transdermal products with each narrowing step of the process. It is understood that if any narrowing step in the work flow causes all the formulations in the pool to be removed, the procedure may be restarted by returning to 14 and generating a new library of formulations containing compositions that have not been previously studied or, alternatively, returning to step 12 and selecting a different set of chemical penetration enhancers with which to work.

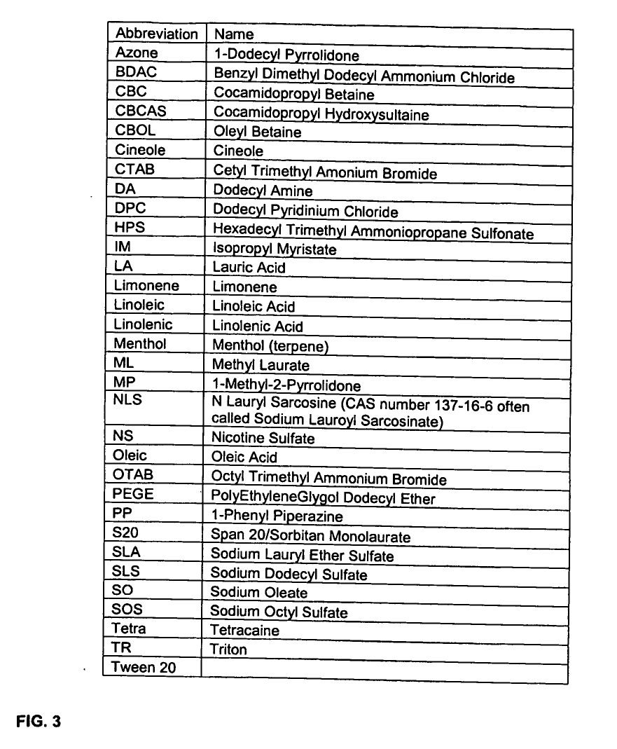

More particularly, a set of CPEs may be chosen, for example by selecting compounds from the list of enhancers introduced previously. CPEs may also be selected from compounds that are analogs of the previously introduced enhancers, or that may be generally classified as, for example, surfactants, azones, solvents, fatty alcohols, fatty acids or fatty esters or selected from compounds that are related to compounds in these classes. The CPEs may also be selected from other compounds that have previously been found to impact skin penetration, or that are related to such compounds. Referring to FIG. 3 a list of CPEs from Example 1 is provided along with their abbreviated names (as used in this specification).

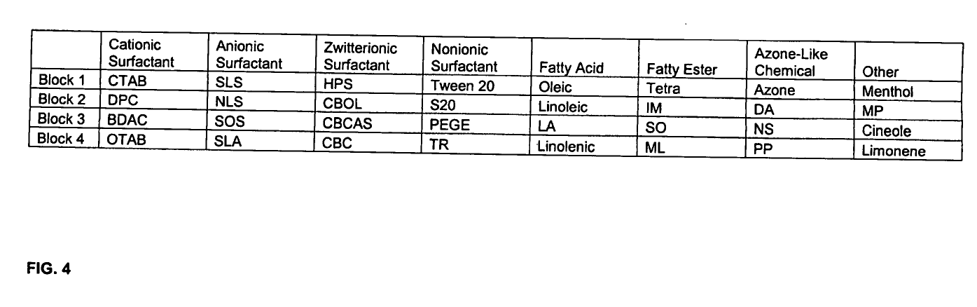

In another embodiment of the present invention a library of CPE formulations is designed. In practical applications CPEs may comprise only a fraction of the composition that is used in a product and it is therefore advantageous to select one or more vehicles to which the CPEs are added to create the CPE formulation as part of a formulation preparation or library design process. The vehicles may be single substances such as, for example, water, an alcohol or other single substance solvent, or may include several substances such as, for example, phosphate buffered saline (PBS) and mixtures of PBS with solvents such as EtOH. The vehicle may also include complex materials designed to mimic the actual use of the CPEs in commercial products such as the matrices used in patch devices, formulations used for cosmetics products and the like. In a preferred embodiment, to assist in later detection of hot spots, the library is designed to include scans over a grid of compositions where the relative concentration of pairs of CPEs are varied, while other compositional variables are held constant. The library may include members that contain, for example, 0, 1, 2, 3, 4, 5 or more different CPEs. Active components that it is desired to deliver topically or transdermally may be either present or absent from members of the library. A practical example of library design is provided in Example 1 below where the set of CPEs introduced in FIG. 3 is divided into subsets, according to their chemical character. Referring to FIG. 4, the CPEs listed in the table in FIG. 3 are classified into 8 separate categories, each category being divided into four blocks to construct the library. The categories are cationic surfactants, anionic surfactants, zwitterionic surfactants, nonionic surfactants, fatty acids, fatty esters, azone-like chemicals, and other.

The fabrication of formulations or libraries may be accomplished entirely manually or with the assistance of automated fluid dispensing systems which are available from a wide range of suppliers (e.g. MultiPROBE® II and MultiPROBE® EX, available from PerkinElmer Life and Analytical Sciences, Inc. of Boston, Mass. (las.Derkinelmer.com), the Multiple Probe 215 and Constellation™ 1200 available from Gilson, Inc. of Middleton, Wis. (www.gilson.com), the Microlab STAR available from Hamilton Company of Reno, Nev. (www.hamiltoncomp.com), the synQUAD available from Genomic Solutions (Cartesian Technologies) of Irvine Calif. (www.cartesiantech.com), the Tango™ available from Matrix Technologies Corp. (Robbins Scientific) of Sunnyvale Calif. (www.robsci.com), and the Genesis and Genesis NPS, available from Tecan, headquartered in Mannedorf near Zurich, Switzerland (www.tecan.com)). In a preferred embodiment of the invention the CPE formulations are fabricated in a sequenced fashion to support screening of the CPE formulations.

The CPE combinations are subjected to screening, for example, HTE screening for a rapid assay of their enhancement potentials. Traditional methods of formulation testing (Franz diffusion cells) rely on steady-state measurements of drug transport across the skin. Bronaugh, 1989. These methods, though useful for quantifying the drug dose delivered across the skin, are not suitable for HTE screening due to: a) inefficient utilization of skin area, b) low time efficiency due to elaborate sample collection and handling, and c) long time periods required to obtain steady state. The HTE method and allied high throughput devices address these challenges. Karande et al. (2002). Other high throughput devices and methods for screening of formulations against skin are set forth in U.S. Pat. No. 5,490,415 and International Application Number PCT/US01/22167 published under International Publication Number WO 02/06518 A1.

In a preferred embodiment of the invention HTE or other screening is accomplished with a high throughput device comprising a donor plate, a receiver plate between which is sandwiched a test membrane which mimics the penetration properties of skin in a living subject. The test membrane may, for example, be human cadaver skin or porcine skin. It may also be a skin model such as the EpiDerm™ skin model available from MatTek Corporation, Ashland, Mass. (www.mattek.com). The donor plate and receptor plate have a series of holes that form donor and receiver compartments for performing measurements of skin penetration.

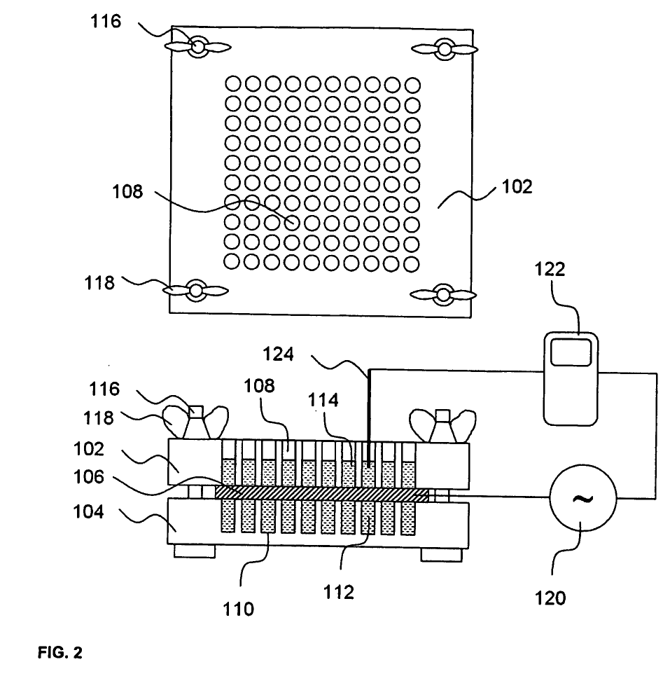

FIG. 2 provides plan and crossectional views an example of a device that may be utilized for high throughput screening. In the plan view at the top of FIG. 2, a donor plate 102 in this example contains 100 donor holes 108. When the device is assembled, one end of the donor holes is sealed by a test membrane 106 to form a series of donor wells also called donor compartments. In typical operation a plurality of samples 114 to be screened is introduced into the donor holes. In the example device presented in FIG. 2 the test membrane 106 is supported by a receptor plate 104 which may also be called a receiver plate. The receptor plate 104 in turn contains a plurality of receptor wells 110 which may also be called receptor compartments, receiver wells or receiver compartments. In normal operation the receiver compartments 110 are filled with a fluid 112. While the use of a receiver plate is generally preferred in such high throughput screening devices the receiver plate is not necessary and other geometries without receiver plates may be utilized, as explained in International Publication Number WO 02/16941 A2. The donor plate 102, receiver plate 104 and test membrane in the device shown in FIG. 2 are secured by means of bolts 116 and wing nuts 118. Where skin conductivity is utilized to monitor changes in the permeability of the test membrane the device may be further provided with one or more electrodes (shown as a single electrode 124 in FIG. 2) for contacting with the samples 114 together with a signal generator 120) and a device 122 for measuring electrical signals, such as for example a digital multimeter.

In a particularly preferred embodiment of the present invention porcine skin may be used as a model for the screening or HTE studies. The donor and receiver plates may be conveniently constructed from materials such as polycarbonate or Teflon and may be approximately one half inch thick. A device, suitable for use in the present invention, may be constructed by drilling 100 holes (each of diameter 3 mm) in the donor and receiver plates to act as the donor and receptor compartments, respectively. Phosphate buffered saline (PBS) may be utilized to fill the receptor compartments and the skin may be clamped between the two plates with the stratum corneum facing the donor plate. Care should be taken to ensure that there are no bubbles between the donor plate and the skin sample, so as to avoid experimental error in later measurements of penetration rates. The donor chambers are used to contain the CPE formulations to be tested.

There are a number of measurements that can be utilized to determine the effect of chemical enhancers on skin permeability. These measurements generally involve contacting the formulation to be tested with skin or other suitable test membrane for a suitable incubation time. The incubation time is preferably in the range of 2-96 hours and more preferably in the range of 4-24 hours. Measurements that may be taken include, for example:
(i) Measurement of solute penetration into the skin: In this approach the ability of a solute or test substance to penetrate into the skin is monitored. Solute diffusion in the SC may be described by Fick's law. The solute concentration in the SC measured at short times is a function of its steady-state permeability. Accordingly, the amount of test substance delivered into the skin can be measured at short times, for example, to screen the efficacy of the enhancers or putative enhancers and formulations containing combinations thereof. The amount of test substance delivered across the skin can also be measured to directly determine the effectiveness of enhancers or putative enhancers and formulations containing combinations thereof. The test substance may take many forms, the only requirement being the availability of a method to measure the amount of the test substance that penetrates into the skin or test membrane. For example, the test substance may be a dye in which case colorimetric measurements can be used to assess the amount of the test molecule penetrating the skin. Alternatively, if test substance concentration can be assessed by HPLC the skin may be solubulized after the incubation period and the resulting solution subjected to HPLC analysis. In yet another embodiment the HTE method follows the transport of a radiolabeled molecule, for example, mannitol, into the skin.
(ii) Skin conductivity: In a preferred embodiment of the invention, electrical conductance may be used to determine skin permeability. Transepidermal current is mediated by the movement of charge carrying ions and is thus related to the permeability of these ions. The ion flux across the skin can be treated in the same way as the flux of solute molecules across the skin. Formal relationships relating ionic conductivity to permeability can be developed using Nemst-Planck flux equations and the Nemst-Einstein relations for ideal solutions. Dugard et al. (1973); Srinivasan et al. (1965). Such relations become significant if one were to precisely estimate skin permeability based on conductivity. However, for screening purposes it is sufficient to know that skin possessing higher electrical conductivity exhibits higher permeability to polar solutes. Accordingly, the electrical conductivity of skin exposed to various compositions is monitored to identify the ones most efficient in increasing skin permeability, specifically to determine the “hot spots” as described herein.
(iii) Concentration changes: The concentrations of compounds in either the donor and/or receptor wells may be monitored as function of time, by periodically sampling of materials from the wells. Changes in concentration as a function of time may be related to the permeability of the sample.

In a preferred embodiment of the invention, skin conductivity is used as endpoints to determine the effect of formulations on skin permeability: Current may, for example, be measured periodically over 24 hrs across the skin at 143 mV peak to peak and 100 Hz frequency. The conductivity enhancement ratio (ER) at time ‘t’ is calculated as ER=Iₜ/I₀, where Iₜ is the current measured at time ‘t’ and I₀ is the current measured at time zero (0). Skin samples occasionally contain defects. It is preferred that precautions are taken to avoid including ER values from wells with defective skin. A simple precaution that may be applied with the setup described here, when porcine skin is used as the test membrane, is to eliminate all ER values for which I₀>3 μA.

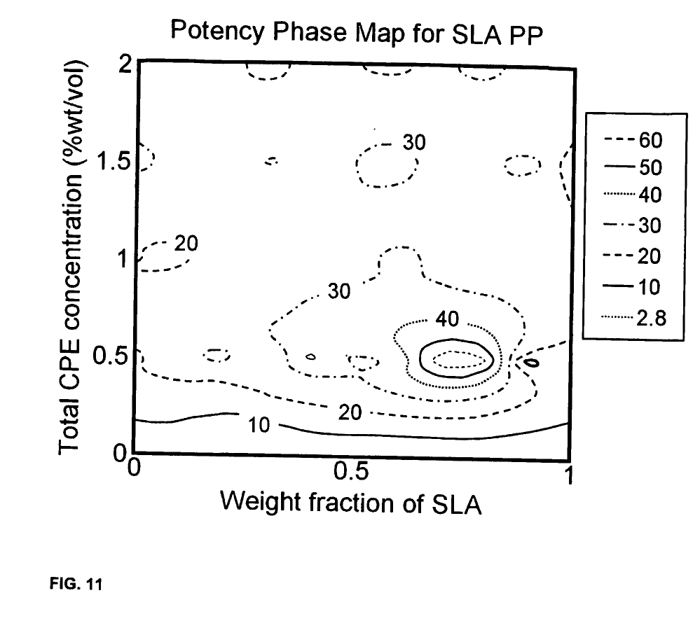

In a preferred embodiment of the invention the data collected from screening or high throughput screening experiments is analyzed for the presence of hot spots. This can be accomplished, for example, by generating potency phase maps, showing skin permeability as the concentrations of two CPEs are varied and looking for sharp maxima with high synergy values in the potency phase maps (concentration of other components being held approximately constant). An example of a potency phase map is provided in FIG. 11 (for further discussion see Example 1).

Measure the irritation potential: A further step is measurement of irritation potential of the hot spot CPE combinations, which can be done by any known method. A variety of in vitro skin corrosion test methods have been developed and several have successfully passed initial international validation. Robinson et al. (2000). These have included skin or epidermal equivalent assays that have been shown to distinguish corrosive from noncorrosive chemicals. These skin/epidermal equivalent assays have also been modified and used to assess skin irritation potential relative to existing human exposure test data. The data show good correlation between the in vitro assay data so developed and different types of human skin irritation data for both chemicals and consumer products. The effort to eliminate animal tests has also led to the development of a novel human patch test for assessment of acute skin irritation potential. A case study shows the benefits of in vitro and human skin irritation tests compared to the animal tests they seek to replace, and strategies now exist to adequately assess human skin irritation potential without the need to rely on animal test methods.

Formulations represented by a hot spot can be placed, 24 at a time, on a culture of human skin cells and the viability of the cells measured at the end of the study period, e.g., 4 to 24 hours, using a MatTek device (MatTek Corporation, 200 Homer Avenue, Ashland, Mass. 01721, www.mattek.com). Human skin constitutes the first immune defense barrier and serves as the interface between the internal milieu and the external environment. Any attempt of using this interface to deliver a formulation is a naturally undesirable perturbation. Cutaneous irritation and corrosion are the main adverse reactions encountered during exposure of skin to a xenobiotic or other external physical agent. Acute irritation can be defined as “a non-immunological, inflammatory, reversible reaction following the applications of a chemical substance to an identical cutaneous site”. Manifestations include inflammation, redness, swelling and pain among other physiological responses. Marzuli et al. (1975); Judge et al. (1996) Wilhelm et al. (2001). Cumulative irritation results from repeated or continued exposure to materials that do not themselves cause acute irritation. Corrosion on the other hand may be defined as “a direct chemical action on skin that results in its disintegration and irreversible alteration at the site of contact”. Manifestations include ulceration, necrosis, and, in time, the formation of scar tissue. Roguet (1999). While the types of cells involved and the clinical aspects of these two reactions are similar, the underlying biological mechanisms are different. Schmitt (1999); Schroder (1995).

Other tools are also available and may be used for determining the irritation potential of the hot spot including, for example, irritation measurements using laser Doppler perfusion imaging, laser Doppler flowmetry, transepidermal water loss, visual scoring, colorimetric measurements, mexameter Hb scale and capacitance measurements. Fluhr et al (2001); Zuang et al. (1999); Ollmar et al (1995). In vitro skin irritation screens and computational approaches have been used an in vitro testing models have been developed using human or animal skin, 3-D skin “equivalent” culture systems derived from human skin cells, or non-cellular “biobarrier” systems. Medina et al. (2000) Lee (2000); Jung et al. (1999); Augustin et al. (1997). A principal computational approach to predicting skin irritation has been “Quantitative Structure Activity Relationship” (QSAR) methodology. Kodithala et al. (2002); Smith et al. (2000); Hayashi et al. (1999). QSAR is based on the evaluation of physicochemical properties of chemical compounds and an attempt to relate these properties to their biological activities. These methods however have been plagued with limitations. Animal and human skin can be difficult to obtain. The equivalent cultures are more permeable to chemicals in absence of the natural pre-epidermal barrier. Bronaugh et al. (1985); Fraissinette et al. (1999). On the other hand QSAR methods are effectively limited to dealing with analog structure-activity training sets while most structure activity data sets consist of structurally diverse compounds. Kodithala et al. (2002). In light of this knowledge and given the low throughput and high expense of irritation potential measurement, methods that make efficient use of irritation potential measurement data are highly desired.

In a preferred embodiment of the invention, in vitro quantification of permeability is performed with respect to formulations that show high penetration ability and low irritation potential. Each identified formulation may be combined with a selected drug or active (if actives are not already present in the library) and each combination may be tested for penetration through skin. This can be done by any known method. For example, a drug-formulation combination can be placed on porcine or human skin and penetration of the drug through the skin can be measured after a period of 24 to 96 hours using Franz diffusion cells (FDC). These results may then compared with published or otherwise available data to determine whether the drug-enhancer formulation can deliver the necessary drug amount.

In vitro quantification of permeability may, for example, be accomplished by means of a vertical Franz diffusion cell with a receptor volume of approximately 12 ml and an area of about 1.7 cm². In such an embodiment 10 μCi/mL radiolabeled mannitol may be used as an exemplary tracer solute in transport experiments. The skin is incubated with the formulations in the FDC assembly. At the end of the incubation period the skin is removed and rinsed gently and the concentration of radiolabelled mannitol is measured using a liquid scintillation counter. Enhancement of transdermal mannitol transport may be calculated as E_{T}=Mₜ/Mₒ, where Mₜ is the amount of mannitol transported after a suitable incubation time in the formulation that showed high penetration ability and M₀ is the amount of mannitol transported in the same incubation time in a control formulation such as PBS.

Animal testing may also be conducted to confirm the ability of the enhancer combinations to deliver sufficient drug or other active across the skin to achieve therapeutic levels of the drug in the animal's blood. For example, in vivo experiments in hairless rats can be performed using leuprolide acetate as a model drug.

Products embodying a SCOPE composition will normally be subject to testing in humans, including irritation and sensitization testing, before being brought to market. One procedure that may be followed for irritation testing is provided by the standardized system described in detail in the paper of Berger and Bowman, based on the earlier work of Lanman et al. Berger et al. (1982); Lanman et al. (1968). In this system test formulations are applied to the skin of the backs of a panel of human volunteers over a 21-day period and 21-day cumulative irritation test score computed by grading reactions to test materials and effects on superficial layers of the skin on a daily basis. The 21-day cumulative irritation test score measured according to Berger and Bowman's system can have a value from 0-630. Test scores can be interpreted as follows:

0-49 indicates a mild material (no experimental irritation);

50-199 indicates a material is probably mild in normal use;

200-449 indicates a material that is possibly mild in normal use;

450-580 indicates a material is an experimental cumulative irritant;

581-630 indicates a material is an experimental primary irritant.

Tests with very similar structure have been provided as guidance to industry by the United States Food and Drug Administration for the irritation testing of generic transdermal drug products. (“Guidance for industry: Skin irritation and sensitization testing of generic transdermal drug products,” U.S. Department of Health and Human Services, Food and Drug Administration, Center for Drug Evaluation and Research, December 1999, available from http://www.fda.gov/cder/guidance/index.htm)

Use of SCOPE Formulations. SCOPE compositions can be utilized in a variety of ways. A SCOPE composition containing the active component of interest may be applied directly to the body surface. Alternatively, two or even more compositions can be applied to the body surface and allowed to mix either passively by diffusion or by means of mechanical agitation to create a SCOPE formulation in situ on the body surface. SCOPE formulations may be applied to a predetermined area of the skin or other tissue for a period of time sufficient to provide the desired local or systemic effect. The method may involve direct application of the SCOPE formulation(s) as an ointment, gel, cream, or the like, or may involve use of a drug delivery device such as a “patch.” Example 3, below, provides one illustration of how a SCOPE formulation can be developed into a gel and utilized in a patch type of device. SCOPE formations may also be used in combination with other approaches for permeabilizing skin including, for example, techniques such as sonophoresis, iontophoresis and electroporation. Mitragotri, 2000, “Synergistic effect . . . ”

Suitable SCOPE formulations for delivery of active components include ointments, creams, gels, lotions, pastes, and the like. Ointments, as is well known in the art of pharmaceutical formulation, are semisolid preparations that typically may be based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should preferably be inert, stable, nonirritating and nonsensitizing. As explained in Remington: The Science and Practice of Pharmacy, 19th Edition ointment bases may be grouped in four classes: oleaginous bases; emulsifiable bases; emulsion bases; and water-soluble bases. Gennaro (1995). Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid.

Creams, also well known in the art, are generally viscous liquids or semisolid emulsions, usually either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels are generally semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions, which are typically preferred for delivery of cosmetic agents, are preparations to be applied to the skin surface with low friction, and are typically liquid or semiliquid preparations in which solid particles, including the active agent, are present in a water or alcohol base. Lotions are usually suspensions of solids. Lotions are preferred formulations for treating large body areas, because of the ease of applying a more fluid composition. In general the insoluble matter in a lotion is finely divided. Lotions will typically contain suspending agents to produce better dispersions as well as compounds useful for localizing and holding the active agent in contact with the skin, e.g., methylcellulose, sodium carboxymethylcellulose, or the like.

Pastes are generally semisolid dosage forms in which the active agent is suspended in a suitable base. Depending on the nature of the base, pastes are often divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base.

Various additives, known to those skilled in the art, may be included in topical formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, e.g., to inhibit growth of microbes such as yeasts and molds. Suitable antimicrobial agents are typically selected from the group consisting of the methyl and propyl esters of p-hydroxybenzoic acid (e.g., methyl and propyl paraben), sodium benzoate, sorbic acid, imidurea, and combinations thereof.

The concentration of the drug or other active component in the formulation can vary a great deal, and will depend on a variety of factors, including the disease or condition to be treated, the nature and activity of the active agent, the desired effect, possible adverse reactions, the ability and speed of the active agent to reach its intended target, and other factors within the particular knowledge of the patient and physician. Preferred formulations will typically contain on the order of about 0.001 wt. % to 50 wt. %, often about 0.01 wt. % to 1.0 wt. %, active component.

An alternative and preferred method of utilizing SCOPE compositions involves the use of a drug delivery system, e.g., a topical or transdermal “patch,” wherein the active agent is contained within a laminated structure that is to be affixed to the skin. Williams (2003). In such a structure, the drug composition is contained in a layer, or “reservoir,” underlying an upper backing layer. The laminated structure may contain a single reservoir, or it may contain multiple reservoirs.

In one embodiment, the reservoir comprises a polymeric matrix of a pharmaceutically acceptable adhesive material that serves to affix the system to the skin during drug delivery; typically, the adhesive material is a pressure-sensitive adhesive (PSA) that is suitable for long-term skin contact, and which should be physically and chemically compatible with the drug or other active agent, chemical penetration enhancers, and any carriers, vehicles or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene(polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing the drug or other active component or other components of the SCOPE formulation contained within the device. The backing is preferably comprised of a flexible elastomeric material that serves as a protective covering to prevent loss of the active component and/or vehicle via transmission through the upper surface of the patch, and will preferably impart a degree of occlusivity to the system, such that the area of the body surface covered by the patch becomes hydrated during use. The material used for the backing layer is typically constructed to permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. Examples of materials useful for the backing layer are polyesters, polyethylene, polypropylene, polyurethanes and polyether amides.

During storage and prior to use, the laminated structure includes a release liner. Immediately prior to use, this layer is removed from the device so that the system may be affixed to the skin. The release liner should be made from a drug/vehicle impermeable material, and is a disposable element that serves to protect the device prior to application. Typically, the release liner is formed from a material impermeable to the active component and other components of the SCOPE formulation, and which is easily stripped from the transdermal patch prior to use.

In another embodiment, the drug and SCOPE-containing reservoir and skin contact adhesive are present as separate and distinct layers, with the adhesive underlying the reservoir. In such a case, the reservoir may be a polymeric matrix as described above. Alternatively, the reservoir may be comprised of a liquid or semisolid formulation contained in a closed compartment or “pouch,” or it may be a hydrogel reservoir, or may take some other form. Hydrogel reservoirs are particularly preferred. As will be appreciated by those skilled in the art, hydrogels are macromolecular networks that absorb water and thus swell but do not dissolve in water. That is, hydrogels contain hydrophilic functional groups that provide for water absorption, but the hydrogels are comprised of crosslinked polymers that give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof. Particularly preferred hydrophilic polymers are copolymers of HEMA and polyvinylpyrrolidone.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which one or more components permeates out of the device. The one or more components may be a drug, a SCOPE formulation, one or more components of a SCOPE formulation, one or more penetration enhancers, or some other component(s) contained in the drug delivery system.

A rate-controlling membrane, if present, will be included in the system on the skin side of one or more of the drug reservoirs. The materials used to form such a membrane are selected to limit the flux of one or more components contained in the drug formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal device, i.e., the skin contact area, has an area in the range of about 5 cm² to 200 cm², preferably 5 cm² to 100 cm², more preferably 20 cm² to 60 Cm². That area will vary, of course, with the amount of drug to be delivered and the flux of the drug through the body surface. Larger patches will be necessary to accommodate larger quantities of drug, while smaller patches can be used for smaller quantities of drug and/or drugs with SCOPE compositions that exhibit a relatively high permeation rate.

Such drug delivery systems may be fabricated using conventional coating and laminating techniques known in the art. For example, adhesive matrix systems can be prepared by casting a fluid admixture of adhesive, the active component, chemical penetration enhancers and a suitable vehicle onto the backing layer in order to form a SCOPE formulation, followed by lamination of the release liner. Similarly, the adhesive mixture may be cast onto the release liner, followed by lamination of the backing layer. Alternatively, the drug reservoir may be prepared in the absence of drug or excipient, and then loaded by “soaking” in a drug/SCOPE formulation mixture. In general, transdermal systems of the invention are fabricated by solvent evaporation, film casting, melt extrusion, thin film lamination, die cutting, or the like.

As with the topically applied formulations of the invention, the SCOPE composition containing the active agent within the drug reservoir(s) of these laminated systems may contain a number of components and generally the drug or other active component will be dissolved, dispersed or suspended together with the synergistic combination of chemical penetration enhancers in a suitable pharmaceutically acceptable vehicle, typically a solvent or gel. Other components which may be present include preservatives, stabilizers, and the like.

### EXAMPLE 1

A library of CPE combinations was developed using the thirty-two individual CPEs listed in the right hand column of the table shown in FIG. 3. The thirty-two CPEs listed in FIG. 3 are referred to as “library CPEs” in Example 1, Example 2, Example 3 and Example 4. Each of the library CPEs was assigned an abbreviated name as shown in the left hand column of the table in FIG. 3, to facilitate tracking and analysis of data. The library CPEs were assigned to one of eight general categories, with four of the CPEs in each. The eight categories and their CPEs were: (i) cationic surfactants (cetyl trimethyl ammonium bromide, dodecyl pyridinium chloride, benzyl dimethyl dodecyl ammonium chloride, octyl trimethyl ammonium bromide); (ii) anionic surfactants (sodium dodecyl sulfate, n-lauryl sarcosine (CAS number 137-16-6 also called sodium lauroyl sarcosinate), sodium octyl sulfate, sodium lauryl ether sulfate), (iii) zwitterionic surfactants (hexadecyl trimethyl ammoniopropane sulfonate, cocamidopropyl betaine, cocamidopropyl hydroxysultaine, oleyl betaine); (iv) nonionic surfactants (Tween 20, Span 20/sorbitan monolaurate, polyethyleneglycol dodecyl ether, Triton); (v) fatty acids (oleic acid, linoleic acid, lauric acid, linolenic acid); (vi) fatty esters (tetracaine, isopropyl myristate, sodium oleate, methyl laurate); (vii) azone-like chemicals (1-dodecyl pyrrolidone, dodecyl amine, nicotine sulfate, 1-phenyl piperazine); and (viii) other (menthol, 1-methyl-2-pyrrolidone, cineole, limonene). The classification of the library CPEs into the eight categories is shown in the table in FIG. 4, using the abbreviated names for the CPEs introduced in FIG. 3.

A library of CPE combinations was constructed from the thirty-two individual CPEs as follows. The CPEs were first divided into four blocks (labeled Block 1, Block 2, Block 3 and Block 4) such that each block had one representative from each of eight categories. The assignment of individual CPEs into the blocks is shown in FIG. 4. The CPEs within each block were then paired to generate all possible distinct binary combinations (yielding 28 binary combinations per block and providing a total of 4×28=112 combinations in the entire library). A compositional grid was then constructed for each pair of CPEs. For each pair of CPEs four different total concentrations of 0.5, 1.0, 1.5 and 2.0% weight/volume were selected. At each total concentration the weight fraction of one enhancer was varied from 0 to 1 in steps of 0.1. Thus for each enhancer pair 44 test formulations were generated yielding a total library containing over 4,000 formulations. All formulations were prepared in a vehicle consisting of 1:1 PBS:EtOH. The PBS solution was 0.01M calcium phosphate, 0.027M potassium phosphate and 0.137M sodium chloride. Formulations were prepared by hand without the use of a robot.

Members of the library were screened for their ability to enhance the penetration of skin in a series of experiments as follows. Porcine skin was used as a model for skin in all the experiments. Skin was harvested from Yorkshire pigs and was stored at −70° C. immediately after procurement until the time of experiments using the methods described in Mitragotri et al., 2000. A high throughput screening device of the type described in International Publication Number WO 02/16941 A2 was utilized to screen the formulations. The apparatus consisted of a polycarbonate plate that served as the donor plate and a Teflon plate that served as the receptor plate. Each plate was 12.7 mm thick. The donor contained a square matrix of 100 wells (each 3 mm in diameter) that served as individual donor compartments. The center-to-center distance between the donor compartments was 6 mm. A matching matrix of 100 wells in the Teflon plate served as individual receptors. The receptor wells were filled with PBS to keep the skin hydrated over the entire duration of the experiment (24 hrs). Skin was thawed at room temperature prior to each experiment. The skin was then placed between the two plates with the stratum corneum facing the donor plate. Donor and receptor plates were clamped together using 4 screws. The skin was incubated with 85 μL of each test formulation in the donor wells for a period of 24 hrs with each formulation being repeated in at least four wells.

The skin penetration enhancement achieved by each formulation was assayed using skin conductivity following the methods disclosed in International Publication Number WO 02/16941 A2. Skin impedance in each well was recorded using two electrodes. One electrode was inserted into the dermis and served as a common electrode while the second electrode was placed sequentially by hand into each donor compartment. An AC signal, 143 mV peak to peak at 100 Hz, was applied across the skin with a waveform generator (Agilent 33120A, Palo Alto, Calif.). Conductivity measurements were performed using a multimeter (Fluke 189, Everett, Wash.) with a resolution of 0.01 μA. Current measurements were performed at two time points, time t=0 (I₀) and time t=24 hrs (I₂₄). The AC signal was only applied while conductivity measurements were being made. The conductivity enhancement ratio (ER) for each formulation was then calculated by taking the ratio of skin conductivities at 24 and 0 hours. The enhancement ratio obtained for each formulation was assigned a unique integer experiment index for subsequent tracking and analysis purposes. In addition, data points from individual wells where the initial current was greater than 31A were assumed to indicate that the skin area between the donor and receptor wells was defective. These data points were discarded and not used in subsequent analysis. Additional experiments were performed when data points were discarded to ensure that there were at least 4 good values of ER available for each formulation.

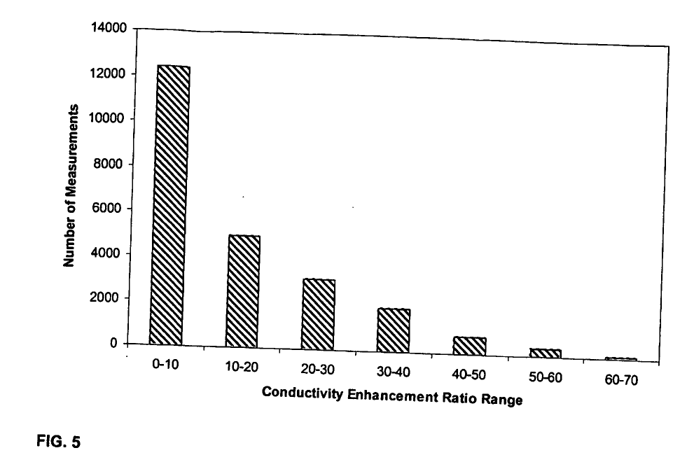

FIG. 5 shows a histogram for over 20,000 separate 24-hour conductivity enhancement ratios that were obtained using the high throughput experimentation approach. Enhancement ratio ranges are plotted horizontally, while the vertical axis shows the frequency with which each given range of enhancement ratios was observed. It can be seen that the frequency with which each range of ER values is observed falls of rapidly as the ER range increases. However, with this large data set very high values of ER are occasionally observed.

The enhancement ratios for the repeat measurements that were made on each formulation were averaged before further analysis and errors in the conductivity enhancement ratio computed using standard statistical formulae.

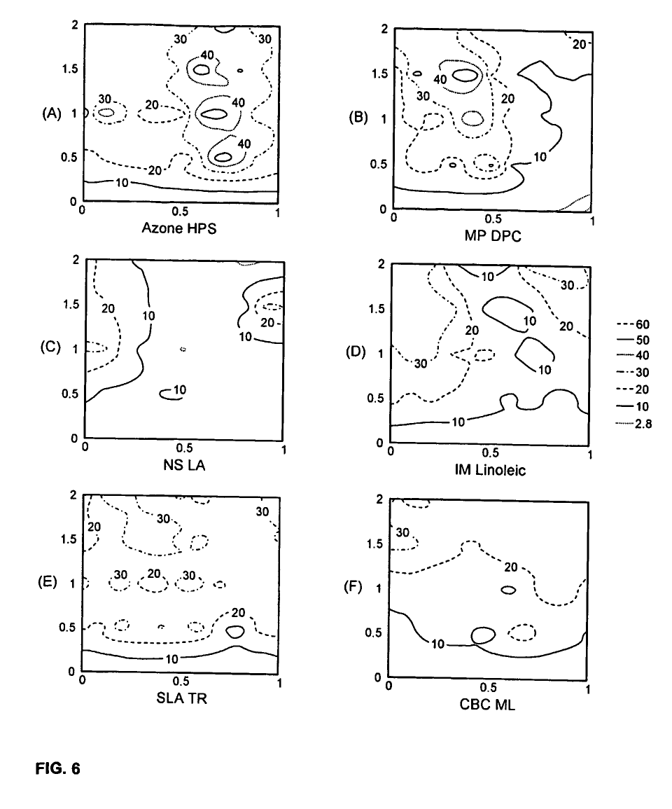

The 44 averaged enhancement ratios generated from each binary combination of CPEs were used to generate two-dimensional contour maps, which may be termed potency phase maps. The six panels in FIG. 6 show potency phase maps for the following pairs of library CPEs: (A) Azone HPS, (B) MP DPC(C)NS LA, (D) IM Linoleic, (E) SLA TR and (F) CBC ML (using the abbreviated chemical names introduced in the table in FIG. 3). In each potency phase map the vertical axis scans the total concentration of library chemical penetration enhancer in units of % weight/volume. The horizontal axis scans the weight fraction of the first named library CPE in the legend below the potency phase map. Thus the left and right hand axes of FIG. 6 (A) provides information about the penetration enhancement effects of HPS in the absence of Azone and Azone in the absence of HPS, respectively. The contour levels show interpolated values of the enhancement ratio based on the 44 data points available for each sample according to the scale inset on the right hand side of FIG. 6.

A range of different interaction behaviors between the library CPEs was observed by analyzing the screening data using potency phase maps. For example, in FIG. 6 (A) and FIG. 6 (B) generally positive synergy can be seen in the potency phase maps; most combinations of Azone HPS and MP DPC give enhancement ratios that are higher than the enhancement ratios obtained from the individual end member CPEs of the formulation at the same total concentration. FIG. 6 (C) and FIG. 6 (D) show examples of generally negative synergy. In these measurements the enhancement ratio of combinations of CPEs was usually lower than that obtained by using the pure end member CPEs at the same concentration. FIG. 6 (E) and FIG. 6 (F) show examples where little synergy is seen between the CPEs.

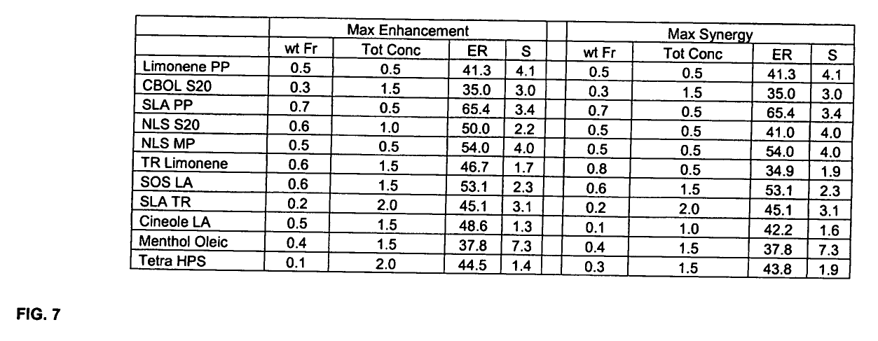

The data were further analyzed to select promising CPE combinations for further analysis. For each formulation a synergy value, S, was

$$S = \frac{{ER}_{A + B} ( X , Y )}{X \cdot {ER}_{A} ( Y ) + ( 1 - X ) \cdot {ER}_{B} ( Y )} ,$$

where ER_{A+B}(X,Y) is the enhancement ratio obtained with the formulation containing CPEs A and B, Y stands for the total concentration of A and B (wt/vol), X stands for the amount of A in formulation (expressed in wt/vol) divided by Y and ER_{A}(Y) and ER_{B}(Y) are the enhancement ratios obtained with pure components A and B at concentration Y (wt/vol) in the vehicle of 1:1 PBS:EtOH, respectively. The data were analyzed to determine the highest observed values of S and the highest observed value of ER for each binary combination of penetration enhancers in the study. CPE combinations that give rise to high sharp maxima in the ER in the potency phase maps have been discovered to produce compositions with low irritation potential. CPE combinations showing high maximum values of ER are expected to be the most promising increasing the permeability of skin. CPE combinations with a tendency to fit these hot spot attributes were selected by choosing formulations that showed (i) large maximum value of ER (ii) large maximum value of S and (iii) small distance in composition space between the maximum value of ER and the maximum value of S. Eleven formulations containing library CPE pairs that were selected for further analysis are shown in FIG. 7. The left hand column lists the binary pairs CPEs. The columns headed Max Enhancement report the position in composition space, observed ER and S value at the maximum ER position in the potency phase map for each pair of library CPEs. The composition position is given as the weight fraction (column headed wt Fr) of the first library CPE to be listed in the first column of the table and a total concentration of the two library CPEs (column headed Tot Conc) expressed in percent weight/volume. Similarly, the columns headed Max Synergy report the position in composition space, observed ER and S value at the maximum S position in the potency phase map for each pair of CPEs.

Experiments to assess irritation potential were performed on the binary pairs of library CPEs at the compositions that yielded the maximum ER value in the screening experiments using the methyl thiazol tetrazolium (MTT) uptake assay. Irritation potential was estimated using EpiDerm™ (MatTek Co., MA, USA, www.mattek.com), a cell culture of normal human derived epidermal keratinocytes. EpiDerm™ cultures were stored and handled according to the standard protocol MTT-ET-50 (MatTek Co., MA, USA, www.mattek.com). To study the effect of the test formulations on cell viability, cell cultures were exposed to 10 μl of each test formulation for 4 hrs. Each test formulation was analyzed in duplicate. At the end of 4 hrs the assay medium was removed and stored aside at −70° C. for Interleukin-1α assay. The cell cultures were rinsed clear of the test formulations using PBS and incubated with 300 μL of MTT reagent (MatTek Co., MA, USA) for 3 hrs at 37° C. and 5% CO₂. At the end of the incubation period the cell cultures were treated with 2 ml of extracting media (provided by MatTek Co) for 2 hrs. 200 μL of this extraction media was then sampled and its optical density (or absorbence) was measured at 570 nm wavelength. The optical absorbance data was then used to calculate the percentage cell viability as recommended in the MTT-ET-50 protocol. Based on the cell viability, the irritation potential (IP) may be defined as follows:

$$IP = 100 ( 1 - \frac{\% cell viability with the formulation}{maximum \% cell viability} ) .$$

The vehicle of 1:1 EtOH:PBS was used as negative control and 1% Triton X-100 was used as the positive control.

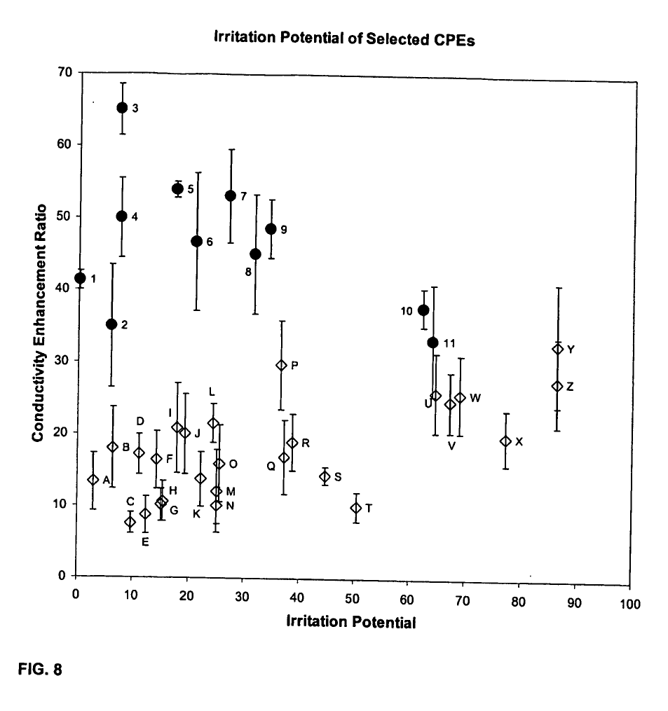

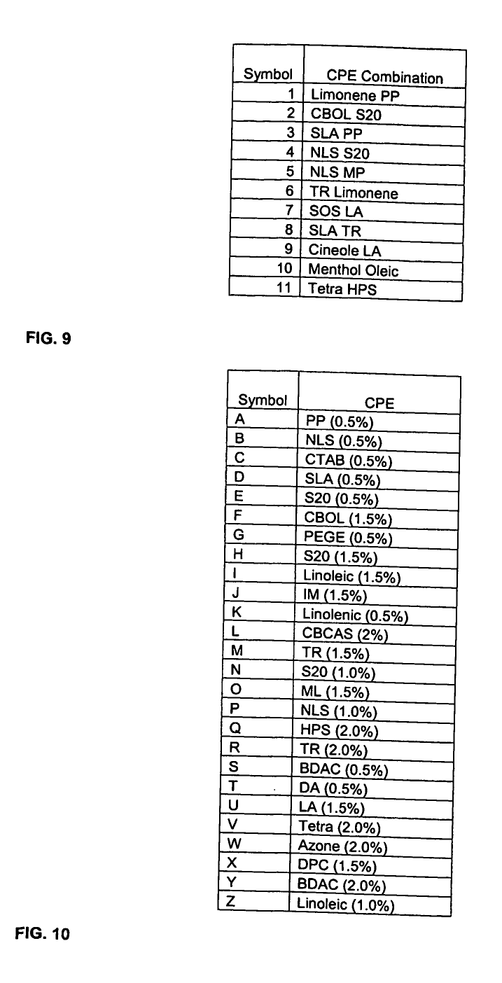

Results obtained in the screening experiments are reported in FIG. 8. The values of IP are plotted on the horizontal axis, while 24-hour ER values are plotted vertically. Solid circles are used for data from formulations containing binary pairs of library CPEs. The library CPEs for each data point may be found by reference to the table given in FIG. 9. Also plotted on FIG. 8, using open diamond symbols, are 24-hour ER and IP values of a number of formulations containing a single library CPE, each labeled with a single letter. The composition of the formulations containing a single library CPE may be found by reference to the table in FIG. 10.

It can be seen that high values of enhancement ratio of the formulations containing a single library CPE shown in FIG. 8 have a tendency to be associated with high values of irritation potential. Formulations representing hot spots selected for analysis following the method of the present invention generally provided higher enhancement ratios and lower irritation potential than the formulations containing a single library CPE. In certain rare cases, such as points 3 and 4 on the chart (corresponding to formulations containing SLA PP and NLS S20, respectively), high ER values and low IP values were achieved simultaneously by the formulations. These formulations were found to have IP values below 10% and ER values above 50. The IP values of these formulations lie below those of 1.5% oleic acid wt/vol in a vehicle of 1:1 PBS:EtOH, which was measured to have an IP value of 14.5%. Oleic acid under these conditions is generally recognized as safe and is used in commercial estradiol patches.

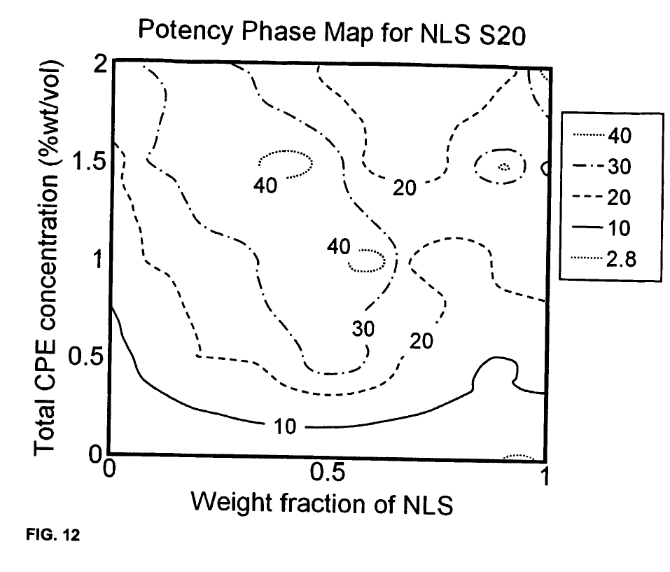

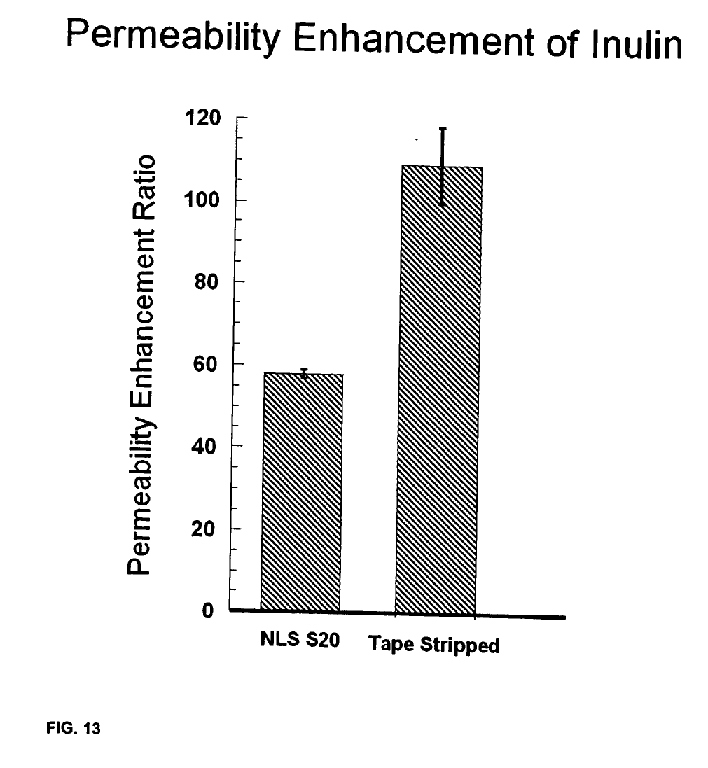

The potency phase map that was measured for SLA PP is shown in FIG. 11 below. A very high sharp maximum, corresponding to a hot spot, can be seen in the potency phase map. The potency phase map that was measured for NLS S20 is provided in FIG. 12.

As discussed previously, without being bound by theory, the low irritation of SCOPE formulations containing binary pairs of library CPEs compared to their formulations containing the individual library CPEs may be based on their relative dynamics in stratum corneum. Due to differential retention of various components in the SC, every stratum in the skin exposed to the formulation experiences a different composition of enhancers. For example, in vitro experiments performed using EpiDerm™ and two model enhancers Sodium Lauryl Sulfate (SLS) and Oleic acid (Oleic) in a vehicle, revealed that the ratio of Oleic:SLS in the epidermis is about 10-times smaller than that in the formulation that was contacted with the SC.

In vitro quantification of the absolute permeability of NLS S20 was confirmed using a Franz diffusion cell (FDC). Bronaugh (1989). Penetration experiments were performed using ³H labeled inulin, (American Radiolabeled Chemicals, St. Louis, Mo.) as a model permeate. FDC's (16 mm diameter, 12 ml receptor volume) were used to assess flux of the radiolabeled inulin across skin. Simultaneous conductivity measurements were performed in the FDC to validate the results from HTS. A small stir bar and an Ag/AgCl disk electrode (E242, InVivo Metric, Ukiah, Calif.) were added to the receptor chamber. The conductivity measurement assembly used was the same as that used in the case high throughput screening experiments except that an Ag/AgCl electrode was used in the receptor compartment instead of inside the skin. The electrical resistance of the electrodes used in both the systems was verified to be similar. The receptor chamber was filled with PBS. Pigskin was thawed and was mounted on the diffusion cell using a clamp with the stratum corneum side facing the donor. Before each experiment the structural integrity of the skin sample was confirmed by measuring its conductivity using the methods set out in Mitragotri et al. (1996). Skin samples with a resistivity of less that 20 kΩ cm² were assumed to be defective and were not used.

An NLS S20 formulation was prepared in PBS (omitting any EtOH) and radiolabled inulin (10 μCi/ml) using the concentration and weight fractions of NLS and S20 that were found to give the maximum ER value in the earlier high throughput screening experiments. The performance of the formulation was compared against a control containing just PBS and radiolabeled inulin a molecule with a molecular weight of about 5,000 Daltons. In addition, experiments were performed using a formulation consisting of PBS and inulin, where the stratum corneum of the skin sample had been removed by tape stripping. See generally, Bronauch and Maibach (1989).

Skin was incubated with radiolabeled test formulation (10 μCi/ml) in the donor for a period of 96 hrs during which time the receptor compartment was sampled periodically. Concentration of radiolabeled solute was measured using a scintillation cocktail and a scintillation counter (Packard Tri-Carb 2100 TR, Meriden, Conn.). Skin permeability was calculated using the standard equations. Permeabilities were corrected to take account of the amount of drug deposited in the skin. The amount of inulin in each skin specimen was measured by gently washing the skin at the conclusion of the FDC experiment, dissolving the skin specimen in Solvable™, a tissue solubilizer, held at about 60° C. for about 12 hours and measuring the concentration of radiolabeled molecules in the resulting solution. Enhancement of permeability was calculated by determining the ratio of permeabilities obtained in the presence and absence of NLS S20. In addition the enhancement of permeability achieved by tape stripping the skin was also computed by determining the ratio of permeabilities obtained from PBS and inulin from tape stripped and intact skin samples. Results are shown in FIG. 13 as a bar chart with the penetration enhancement ratio derived from the FDC measurements plotted vertically. It was found that the NLS S20 hot spot combination was very effective in improving the transport of inulin across the stratum corneum, improving the permeability by more than 50 fold compared with a sample which omitted the CPEs. In addition it was found that performance of the NLS S20 formulation is about 50% of that obtained with tape stripped skin, which models the performance of a CPE combination that is 100% effective in removing the penetration barrier of the stratum corneum.

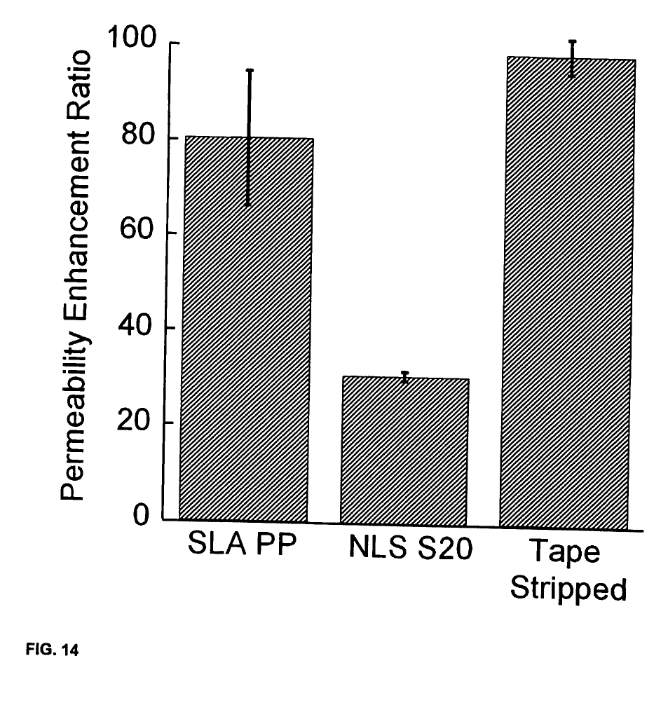

Further FDC measurements on porcine skin to measure transport of radiolabeled inulin were also taken on the NLS S20 combination and the SLA PP combination using a 1:1 PBS:EtOH vehicle using the methods outlined in the previous paragraphs. The NLS S20 formulation utilized a total concentration of library CPE of 1% wt/vol with an NLS library CPE weight fraction of 0.6. The SLA PP formulation utilized a total concentration of library CPE of 0.5% wt/vol with an SLA library CPE weight fraction of 0.7. Also tested was the case of tape stripped skin in the presence of inulin in a PBS vehicle. Radiolabeled inulin was added to all formulations to a level of 10 μCi/ml. Results are reported in FIG. 14. The permeability enhancement ratio was computed in each case by comparisons with the flux rate of inulin in a vehicle consisting of PBS only through intact skin. In contrast to the results shown in FIG. 13, the data in FIG. 14 does not include corrections for the amount of inulin deposited into the skin. The hot-spot formulations containing SLA PP and NLS S20 are both highly effective in promoting the transport of inulin across skin. In the case of SLA PP the permeability enhancement ratio is about 80% of the value observed with tape stripped skin.

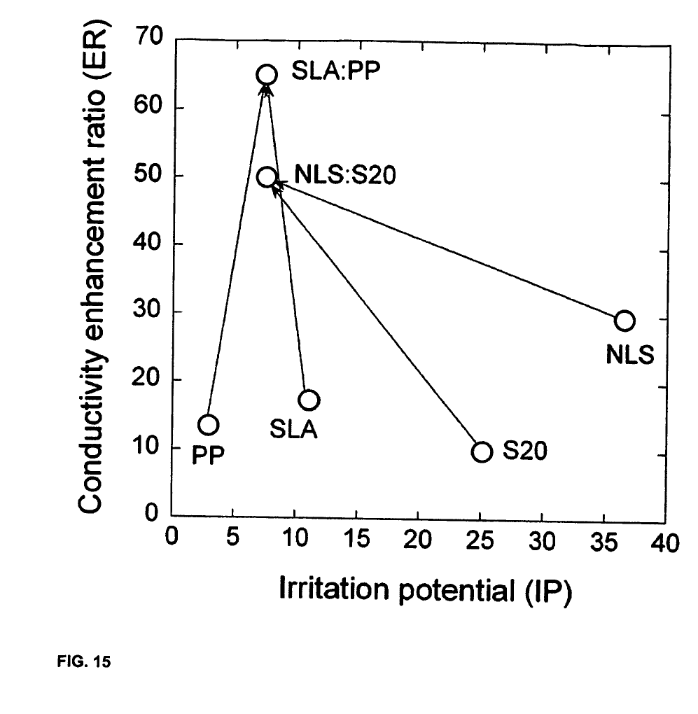

Finally, the irritation potential and conductivity enhancement ratio of the SLA PP and NLS S20 hot-spot formulations are shown in FIG. 15 together with conductivity enhancement ratios and irritation potentials of formulations containing constituent CPEs. All formulations utilized a vehicle of 1:1 PBS:EtOH. The points in FIG. 15 are labeled according to the library CPEs contained within each formulation. The concentrations of library CPE of each of the labeled points in the FIG. 15 are as follows: SLA:PP SCOPE formulation, total library CPE concentration 0.5% wt/vol, SLA weight fraction 0.7; SLA, total library CPE concentration 0.5% wt/vol; PP, total library CPE concentration 0.5% wt/vol; NLS:S20 SCOPE formulation, total library CPE concentration 1% wt/vol, NLS weight fraction 0.6; NLS, total library CPE concentration 1% wt/vol; S20, total library CPE concentration 1% wt/vol. It can be seen that the conductivity enhancement of the SCOPE formulations is substantially enhanced compared with the formulations containing a single library CPE, reflecting the previously discussed synergies in penetration enhancement produced by the library CPEs. In the case of the NLS:S20 SCOPE formulation the irritation potential of the formulation was substantially reduced when compared to that of the formulations containing the individual library CPEs at the same total library CPE concentration. In the case of the NLS:S20 SCOPE formulation the irritation antergy factor, A, defined through

$$A = \frac{X \cdot {IP}_{A} ( Y ) + ( 1 - X ) \cdot {IP}_{B} ( Y )}{{IP}_{A + B} ( X , Y )} ,$$

is substantially greater than 1 (symbols in this equation having the meanings provided earlier in the definition of antergy factor). For the case of the NLS:S20 SCOPE formulation the irritation antergy factor, using values of irritation potential measured using the MTT uptake assay as explained above, is calculated to be 4.2.

### EXAMPLE 2

In vitro FDC experiments were performed to evaluate the ability of formulations containing an SLA:PP SCOPE formulation to enhance the delivery of test molecules with a range of molecular weights across the stratum corneum. Test molecules whose transport properties were measured were mannitol (MW≈180 Da, a small molecule), methotrexate (MW≈454 Da, a small molecule), luteinizing hormone releasing hormone (LHRH, MW≈1.2 kDa, a peptide), inulin (MW=5 kDa, a polysaccharide), low molecular weight heparin (LMWH, MW≈10 kDa, a polysaccharide) and an oligonucleotide (ODN, MW≈˜15 kDa). Concentration changes of the molecules due to transport were measured using radiolabeled chemicals. ³H-labeled forms of the test molecules were obtained from the following sources: mannitol, methotrexate, inulin and LMWH were acquired from American Radiolabeled Chemicals of St. Louis, Mo. (www.arc-inc.com); LHRH was obtained from NEN, now part of Perkin Elmer, Wellesley, Mass. (www.Derkinelmer.com); ODN was provided by ISIS Pharmaceuticals of Carlsbad, Calif. (www.isispharm.com). Each radiolabeled test molecule was directly added to formulation containing the CPEs SLA and PP in a vehicle of 1:1 PBS:EtOH at a concentration of 10 μCi/ml. The total concentration of the library CPEs in the SCOPE formulation was 0.5% wt/vol, the SLA weight fraction of library CPE being 0.7. The resulting formulations were placed in the donor well of Franz cells and the contents of the receiver wells were sampled periodically for a period of 96 hours to monitor transport. FDCs utilized in the experiments had a diameter of 16 mm and receiver volume of 12 ml. Small stir bars and Ag/AgCl disk electrodes (model number E242 acquired from In Vivo Metric, Healdsburg, Calif. (www.invivometric.com)) were added to the receiver chamber, the disk electrode allowing skin conductivity to be measured as the experiment proceeded. The FDC receiver chambers were filled with PBS and adequate measures were taken to prevent inclusion of air in the receiver chamber. Thawed pig skin, harvested from Yorkshire pigs and stored at −70° C. immediately after procurement until the time of experiments using the methods described by Mitragotri et al. was mounted on the diffusion cell using a clamp with the stratum corneum side facing the donor well. Mitragotri et al. (2000). The concentration of the radiolabeled test molecule was measured using a Packard Tri-Carb 2100 TR scintillation counter. FDC measurements were repeated several times for each test molecule to ensure statistically meaningful results. In addition permeabilities were corrected to take account of the amount of drug deposited in the skin. The amount of test molecule in each skin specimen was measured by gently washing the skin at the conclusion of the FDC experiment, dissolving the skin specimen in Solvable™, a tissue solubilizer, held at about 60° C. for about 12 hours and measuring the concentration of radiolabled molecules in the resulting solution.

In order to confirm that detected radioactivity was a result of transport of the test molecules and not from tritiated water that may have resulted from tritium exchange, receiver samples were desiccated and analyzed for radioactivity. No substantial differences in radioactivity were observed between native and desiccated receiver samples.

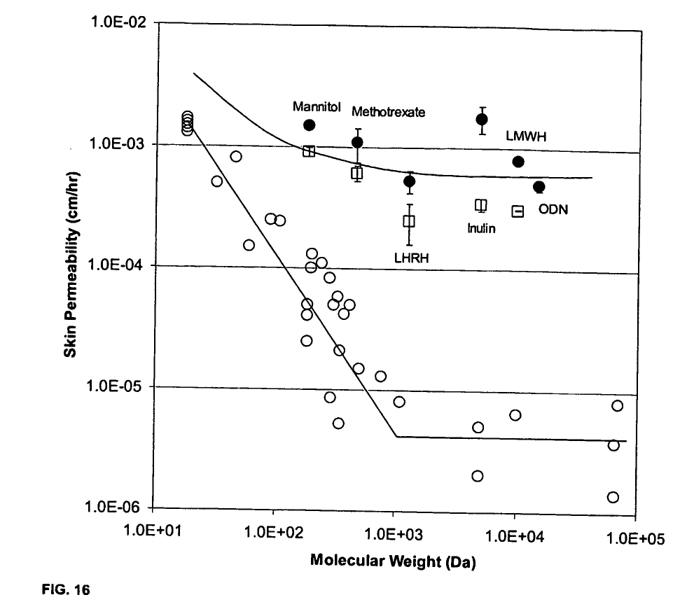

The measured skin permeabilities as measured in the FDC experiments are shown graphically with the open square symbols in the log-log plot in FIG. 16. The closed circles show the permeabilities corrected for amounts of the test molecules deposited in the skin. In the case of ODN, the majority of the oligonucleotides were trapped in the skin and only the permeability value calculated based on amounts deposited in the skin is reported. The open circles in FIG. 16 show permeability of untreated skin reported in the literature for a variety of hydrophilic solutes. Mitragotri (2003). It can be seen that the SLA:PP SCOPE formulation produces substantial increases in the permeability of skin for the test molecules compared to that usually observed for hydrophilic molecules. In addition it can be seen from the present example that the SCOPE formulation containing SLA and PP at relatively low concentration is able to deliver not only small molecule drugs but also larger molecules with the character of peptides, oligonucleotides and polysaccharides. Moreover, it should also be noted that the test molecules of the present example are hydrophilic in character, which are traditionally the most difficult to deliver across the skin barrier.

### EXAMPLE 3

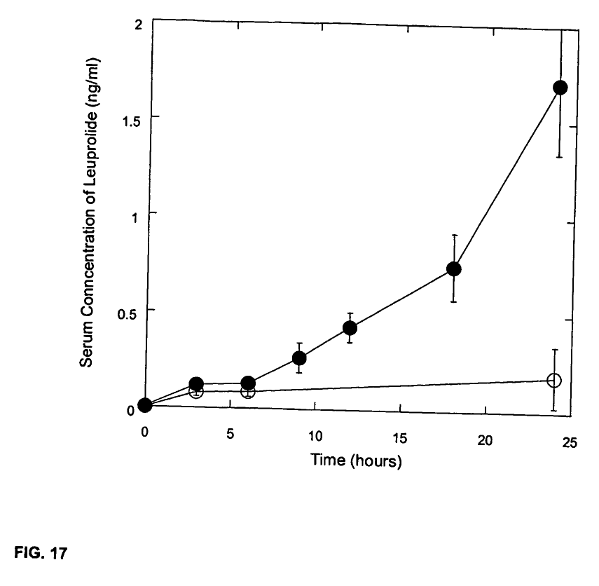

In vivo experiments were performed using hairless rats (250-280 gm) from Charles River Laboratories, Wilmington, Mass. (www.criver.com). All experiments on the animals were performed according to institutionally approved protocols at the University of California, Santa Barbara. Animals were anesthetized using isofluorane (1.25-3% isofluorane in oxygen). 1 gm of a either a control gel containing leuprolide or a gel containing the CPEs SLA and PP and the drug leuprolide was applied to the lateral side of the rat above the left hind leg over a skin area of 9 cm². The control gel utilized 2 mg/ml leuprolide dissolved in PBS containing 1.8% wt/vol hyaluronic acid. The second gel, based on the SLA PP SCOPE formulation discovered in Example 1, contained 2 mg/ml leuprolide, 0.35% wt/vol SLA, 0.15% wt/vol PP and 1.8% wt/vol hyaluronic acid in 1:1 PBS:EtOH. A thin polymer sheet was placed on the gel patches and the edges sealed with a cyanoacrylate adhesive. The animals were allowed to recover from anesthesia after 2 hrs. Blood samples were collected from the jugular vein over a period of 24 hrs and plasma concentration of the leuprolide measured using ELISA (using product number S-1159 from Bachem Bioscience, Bubendorf, Switzerland (www.bachem.com). The results of the experiment are shown in the graph in FIG. 17 with plasma concentration of leuprolide plotted on the vertical axis and time plotted horizontally. Solid and open symbols provide results for plasma concentration of the SLA:PP-containing formulation and control formulation, respectively.

The skin of the rats was observed throughout the experiments. The skin appeared normal throughout the experiment and no erythema was observed at any time.

Skin conductance was measured during the course of the experiment to assess skin permeabilization and recovery. The SLA:PP SCOPE formulation caused a roughly 20-fold increase in skin conductance during the 24 hrs that the formulation was in contact with the skin. The skin conductance fell back to 20% of the peak value within 12 hrs after removal of the formulation.

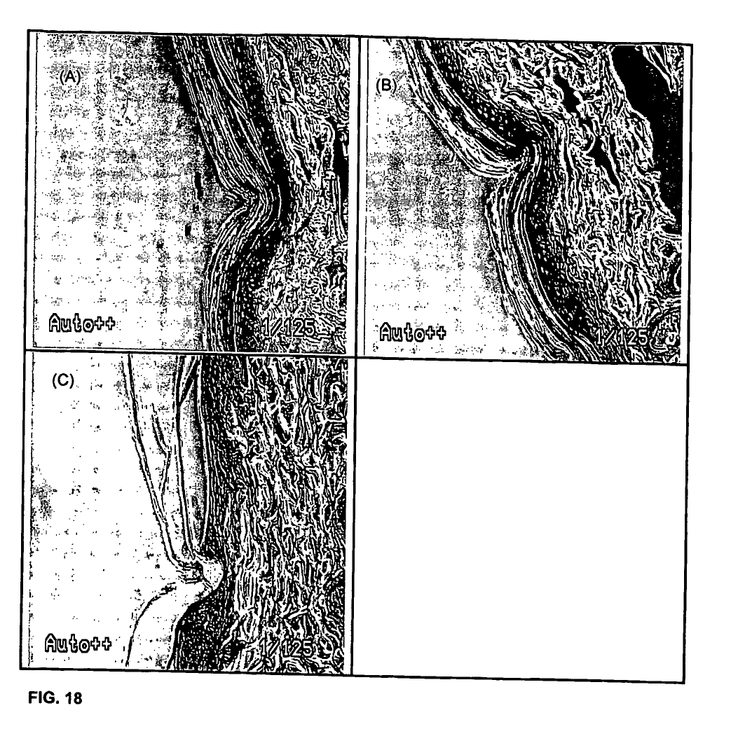

In some animals the skin exposed to the SLA:PP SCOPE formulation and the control formulation was excised and fixed in 10% vol/vol formalin immediately after removing the patch. The skin was sectioned and stained with hematoxylin and cosin by Mass Histology Service, Warwick, R1 (www.masshistology.com). Histological studies showed the stratum corneum of the skin exposed to the SLA:PP formulation to be normal and no structural differences were observed in the skin compared with controls. There were no signs of inflammation in the histological sections and the presence of inflammatory cells was not detected. FIG. 18 (A) is a micrograph of skin section obtained from a hairless rat after application of the PBS/hyaluronic acid based control patch, while FIG. 18 (B) is a micrograph of a skin section from a rat that had received the SLA:PP-containing patch. In addition, patches were also applied to hairless rats utilizing a formulation containing 10% wt/vol SLS, 1.8% wt/vol hyaluronic acid and 2 mg/ml leuprolide made up in a 1:1 PBS:EtOH vehicle, as a positive control. A typical micrograph of the skin section obtained after applying this formulation in a patch to a hairless rat, according to the protocol outlined previously, is provided in FIG. 18 (C). It can be seen that the stratum corneum of the rat has completely detached from the lower lying skin layers, in contrast to what was observed in FIG. 18 (A) and FIG. 18 (B), where the skin remains intact.

Experiments were also performed on hairless rats utilizing a formulation consisting of 2 mg/ml leuprolide, 0.35% wt/vol SLA, 0.15% wt/vol PP and 1.5% wt/vol hydroxypropyl cellulose MF in 1:1 PBS:EtOH. The performance of patches utilizing this formulation were very similar to those obtained with the formulation containing SLA, PP, leuprolide and hyaluronic acid described previously.

### EXAMPLE 4

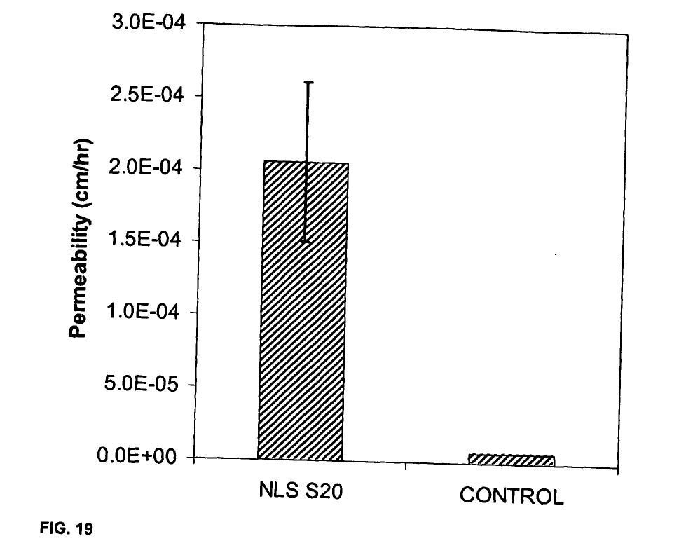

In vitro FDC experiments were performed to compare the flux of corticosterone, a lipophilic molecule (Log K_{o/w}=1.94), across porcine skin using a SCOPE formulation containing NLS S20 in a 1:1 PBS:EtOH vehicle against that obtained with a PBS-based formulation. The total concentration of NLS and S20 in the SCOPE formulation was 1.0% wt/vol and the library CPE weight fraction of NLS was 0.6. Radiolabeled corticosterone was acquired from NEN (now part of Perkin Elmer, Wellesley, Mass. www.perkinelmer.com) and added to the two formulations at a concentration of 10 μCi/ml. FDC experiments were conducted as described in Example 2 using porcine skin. Samples were obtained periodically from FDCs over the entire duration of 96 hrs period in which the skin was exposed to the test formulations. Concentration of radiolabeled solute in these samples was measured with a scintillation counter and the molecular flux and skin permeability were calculated using standard equations as described previously. A permeability enhancement ratio was calculated by taking the ratio of skin permeability to corticosterone at 96 hrs obtained with the SCOPE formulation to that obtained with the PBS based solution. The permeability enhancement ratio obtained in this manner was computed to be 30. FIG. 19 depicts the flux rate of corticosterone across porcine skin in the NLS:S20 formulation and the PBS formulation.

Much evidence points to the fact that molecules with molecular weight greater than 500 Da do not pass through the stratum corneum in significant amounts. Bos et al., (2000). The molecular weights of virtually all common contact allergens, most commonly used pharmacological agents applied in topical dermatotherapy, and all drugs presently available in FDA-approved transdermal patches are less than 500 Da. The lack of effective CPEs has largely restricted pharmaceutical development of new innovative products to those containing drugs with a MW of less than 500 Da when topical dernatological therapy or percutaneous systemic therapy or vaccination is the objective. A predictive rule of thumb that has been applied in the field of transdermal drug delivery is that the maximum flux of drug through the skin decreases by a factor of 5 for an increase of 100 Da in MW. Finnin et al. (1999). Moreover, drugs that are normally considered suitable for transdermal drug delivery should be lipophilic with log K_{o/w} in the range of 1-3. Finnin et al. (1999). In contrast, the examples presented here serve to illustrate that transport of drugs and other active components can be achieved without these restrictions by the use of formulations containing rare combinations of chemical penetration enhancers. The data in Example 1 demonstrates that inulin (a 5,000 Da molecule) is transported well across the stratum corneum using formulations containing low concentrations of NLS and S20, and SLA and PP. Example 2, demonstrates that a range of hydrophilic molecules, spanning molecular weight range from 180 Da ˜15,000 Da, can be delivered across skin utilizing SCOPE formulations. Moreover, in FIG. 16 it can be seen that the usual rule that a decrease of skin penetration by a factor of 5 occurs for each 100 Da increase in molecular weight no longer holds with SCOPE formulations. SCOPE formulations also overcome the restrictions limiting transdermal delivery of lipophilic molecules (log K_{o/w} in the range of 1-3) as illustrated, for example, by the data presented on exemplary test molecules such as mannitol (log K_{o/w}=−3.1) and inulin (log K_{o/w}=−3) in Example 2. Example 4 on the other hand, illustrates that SCOPE formulations may also be used greatly enhance the transport of corticosterone, a low molecular weight lipophilic drug (MW=346.5 Da, log K_{o/w}=1.94), and therefore that SCOPE formulations also have utility in improved delivery of molecules that would conventionally be considered candidates for topical and transdermal delivery.

From the foregoing, it will be appreciated that, although specific embodiments of the invention have been described herein for the purpose of illustration, various modifications may be made without deviating from the spirit and scope of the invention. Accordingly, the present invention is not limited except as by the appended claims.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, the claims referred to above including but not limited to any original claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features reported and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

Citations

Audus, K. L., Bartel, R. L., Hidalgo, I. J. and Borchardt, R. T., The use of cultured epithelial and endothelial cells for drug transport and metabolism studies. Pharm Res. 1990 7(5): p. 435-51.
Augustin, C; Colombel, C; Damour, O., Use of in vitro dermal equivalent and skin equivalent kits for evaluating cutaneous toxicity of cosmetic products. In vitro Toxicology 1997. 10 p. 23-31.
Berger, R. S, and Bowman, J. P., A reappraisal of the 21-day cumulative irritation test in man. J. Toxicol.—Cut. & Ocular Toxicol. 1982 1(2) p 109-115.
Berti, J. and Lipsky J. J., Transcutaneous drug delivery: A practical review. Mayo Clinical Proceedings, 1995. 70: p. 581-586.
Bos J. D., Meinardi M. M. H. M., The 500 Dalton rule for the skin penetration of chemical compounds and drugs. Exp. Dermatol. 2000. 9 p. 165-169.
Bouwstra, J. A., The skin, a well organized membrane. Colloids and Surfaces. A, 1997. 123-124: p. 403-413.
Bronauch R. L. and Maibach H. I., Percutaneous Absorption; Mechanisms-Methodology-Drug Delivery, Marcel Dekker Inc.: New York, Basel, 1989.
Bronaugh, R. L., and Stewart, R. F., Methods for in vitro percutaneous studies absorption studies IV: The flow through diffusion cell. Journal of Pharmaceutical Science, 1985.74: p. 64-67.
Bronaugh, R. L., Determination of Percutaneous Absorption by In vitro Techniques, In Percutaneous Absorption; Mechanisms-Methodology-Drug Delivery, R. L. Bronaugh, Maibach, H. I., Editor, Marcel Dekker Inc.: New York, Basel, 1989.
Dugard, P. H. and Scheuplien, R. J., Effects of ionic surfactants on the permeability of human epidermis: An electrometric study. Journal of Investigative Dermatology, 1973. 60: p. 263-269.
Finnin, C. B. and Morgan, T. M., Transdermal penetration enhancers: Applications, limitations and potential. Journal of Pharmaceutical Sciences, 1999. 88: p. 955-958.
Fluhr, J. L., et al., Testing for irritation with a multifactorial approach: comparison of eight non-invasive measuring techniques on five different irritation types. British Journal of Dermatology, 2001. 145: p. 696-703.
De Brugerolle de Fraisinette, A. et al., Predictivity of an in vitro model for acute and chronic skin irritation (SkinEthic) applied to the testing of topical vehicles. Cell Biology and Toxicology, 1999. 15: p. 121-135.
Faller, C., Bracher, M., Dami, N. and Roguet, R., Predictive ability of reconstructed human epidermis equivalents for the assemssment of skin irritation of cosmetics. Toxicology in vitro, 2002. 16: p. 557-572.
Funke, A. P., Schiller, R., Motzkus, H. W., Günther, C., Müller, R. H. and Lipp, R., Transdermal delivery of highly lipophilic drugs: In vitro fluxes of antiestrogens, permeation enhancers, and solvents from liquid formulations. Pharmaceutical Research 2002. 19: p. 661-668.
Gennaro, A. R., editor. Remington: The Science and Practice of Pharmacy. 19th edition. Pennsylvania: Mack Publishing Company; 1995
Hadgraft, J. Trans(dermal) delivery, present and future perspectives. The Drug Delivery Companies Report Spring/Summer 2003, published by PharmaVentures Ltd. see http://www.iirusa.com/docs/P0950 Hadgraft.pdf.
Hayashi, M., et al., A quantitative relationship study of the skin irritation potential of phenols. Toxicology In vitro, 1999. 13(6): p. 915-922.
Judge, M. R., et al., Variation in response of human skin to irritant challenge. Contact Dermatitis, 1996. 34: p. 115-117.
Jung, S., et al., Skin organ culture for cutaneous irritancy screening. In vitro and molecular toxicology, 1999. 12(2): p. 77-82.
Karande, P. S, and Mitragotri, S., High Throughput Screening of transdermal formulations. Pharmaceutical Research, 2002. 19: p. 655-660.
Kodithala, K., et al., Prediction of skin irritation from organic chemicals using membrane-interaction QSAR analysis. Toxicological Sciences, 2002. 66: p. 336-346.
Lanman, B. M., Elvers, E. B. and Howard C. J., The role of human patch testing in a product development program. Joint Conference on Cosmetic Sciences, The Toilet Goods Association (currently The Cosmetic, Toiletry and Fragrance Association), Washington D.C., Apr. 21-23, 1968.
Lee, J. K., In vitro cytotoxicity tests on cultured human skin fibroplasts to predict skin irritation potential of surfactants. Toxicology In vitro, 2000. 14(4): p. 345-349.
Marzuli, F. N. and Maibach, H. I., The rabbit as a model for evaluating skin irritants: a comparison of results obtained on animals and man using repeated skin exposure. Food Cosmetology and Toxicology, 1975. 13: p. 533-540
Medina, J., et al., Use of human skin equivalent Apligraf for in vitro assessment of cumulative skin irritation potential of topical products. Toxicology and Applied Pharmacology, 2000. 164: p. 38-45.
Mezei, M. and Gulasekharam, V., Liposomes—a selective drug delivery system for the topical route of administration. Lotion dosage form. Life Sci. 1980. 26(18): p. 1473-1477.
Mitragotri, S., et al., Transdermal drug delivery using low-frequency sonophoresis. Pharmaceutical Research, 1996. 13: p. 411-420.
Mitragotri, S., Synergistic effect of enhancers for transdermal drug delivery. Pharmaceutical Research, 2000. 17: p. 1354-1359.
Mitragotri, S., et al., Determination of the threshold energy dose for ultra-sound-induced transdermal drug delivery. J. Control. Release 63:41-52 (2000).
Mitragotri, S. Modeling skin permeability to hydrophilic and hydrophobic solutes based on four permeation pathways. J. Control. Release 86:69-92 (2003).
Mollgaard, B., Synergistic effects in percutaneous enhancement. Pharmaceutical Skin Penetration Enhancers, ed. K. Walters and J. Hadgraft. 1993, New York, Basel, Hong Kong: Marcel Dekker. 229-242.
Ng, S., Goodson, B., Ehrardt, R., Moos, W. H., Siani, M. and Winter, J., Combinatorial Discovery Process Yields Antimicrobial Peptides. Prog. Med. Chem. 7: 1781-5 (1999).
Ollmar, S., et al., Electrical impedance for estimation of irritation in oral mucosa and skin. Medical Progress Through Technology, 1995. 21(1): p. 29-37.
Mak, V. H. W. and Francoeur, M. L., Diffusion test apparatus and method. U.S. Pat. No. 5,490,415. Date of Patent: Feb. 13, 1996.
Robinson M. K., Osborne R. and Perkins M. A., In vitro and human testing strategies for skin irritation. Ann N Y Acad. Sci. (2000) 919:192-204.
Roguet, R., Use of skin cell cultures for in vitro assessment of corrosion and cutaneous irritancy. Cell Biology and Toxicology, 1999. 15: p. 63-75.
Santus, C. G. and Baker, R. W., Transdermal enhancer patent literature. Journal of Controlled Release 1993. 25: p. 1-20.
Schmitt, D., Immune functions of the human skin. Models of in vitro studies using Langerhans cells. Cell Biology and Toxicology, 1999. 15: p. 41-45.
Schroder, J. M., Cytokine networks in the skin. Journal of Investigative Dermatology, 1995. 105(1): p. S20-S24.
Shah, V. P., Peck, C. C. and Williams, R. L., Skin Penetration Enhancement: Clinical pharmacological and regulatory considerations. Pharmaceutical skin penetration enhancement, ed. K. Walters. 1993, New York, Basel, Hong Kong: Marcel Dekker. 417-427.
Smith, J. S., et al., Experimental Validation of a structure-activity relationship model of skin irritation by esters. Quantitative structure activity relationships, 2000. 19(5): p. 467-474.
Srinivasan, V. and Higuchi, W. I., A model for iontophoresis incorporating the effect of convective solvent flow. International Journal of Pharmaceutics, 1965. 60: p. 133-138.
Verdine, G. L., The Combinatorial Chemistry of Nature. Nature. 384: 11-13 (1996).
Walters, K. A., Penetration enhancers and their use in transdermal therapeutic systems. Transdermal Drug Delivery. Developmental Issues and Research Initiatives, ed. J. Hadgraft and R. H. Guy. 1989, New York: Marcel Dekker. 197-233.
Wilhelm, K. P., Bottjer, B. and Siegers, P. C., Quantitative assessment of primary skin irritants in vitro in a cytotoxicity model: comparison with in vivo human irritation tests. British Journal of Dermatology, 2001. 145: p. 709-715.
Williams A C, Transdermal and topical drug delivery: From theory to clinical practice. Pharmaceutical Press London, Chicago, 2003.
Zhang, B., et al., Discovery of a Small Molecule Insulin Mimetic with Antidiabetic Activity in Mice. Science. 284: 974-7 (1999).
Zuang, V., et al., Detection of skin irritation potential of cosmetics by non-invasive measurements. Skin Pharmacology and Applied Skin Physiology, 2000. 13(6): p. 358-371.

1. A method for selecting compositions having potent ability to increase the permeability of a body surface as candidates for further testing for low irritation potential, comprising the following steps:
(a) providing a library, said library comprising a plurality of samples, each sample comprising at least two chemical penetration enhancers;
(b) measuring with a high throughput device the abilities of the samples to increase the permeability of a test membrane; and
(c) analyzing the measurements to select compositions having potent ability to increase the permeability of said body surface.

2. The method of claim 1 in which the abilities of the samples to increase the permeability of the test membrane are measured by a method comprising:
(a) securing the test membrane to a device comprising a donor plate, the donor plate including a plurality of donor holes passing through the donor plate;
(b) introducing the samples into donor holes, each sample including a test substance; and
(c) evaluating the amount of the test substance that remains in the donor holes or that migrates into the test membrane after a suitable incubation time.

3. The method of claim 1 in which the abilities of the samples to increase the permeability of the test membrane are measured by a method comprising:
(a) securing the test membrane to a device comprising a donor plate, the donor plate including a plurality donor holes passing through the donor plate;
(b) securing the test membrane to a receiver plate such that the test membrane is disposed between the donor plate and the receiver plate, said receiver plate including a plurality of receiver wells corresponding to the donor holes and said receiver wells containing a liquid;
(c) introducing the samples into donor holes, each sample including a test substance; and
(d) evaluating the amount of the test substance in each donor hole that migrates through the test membrane into the receiver well corresponding to said donor hole.

4. The method of claim 1 wherein the test membrane is mammalian skin or mucosa.

5. The method of claim 1 wherein the abilities of the samples to increase the permeability of the test membrane are measured by making a plurality of electrical conductivity measurements.

6. The method of claim 1 in which each of a plurality of the samples comprises three chemical penetration enhancers.

7. The method of claim 1 in which each of a plurality of the samples comprises four chemical penetration enhancers.

8. The method of claim 1 in which each of a plurality of the samples comprises more than four chemical penetration enhancers.

9. The method of claim 1 in which the library contains more than 1,000 samples.

10. The method of claim 1 in which the library contains more than 10,000 samples.

11. The method of claim 1 in which the library contains more than 1,000,000 samples.

12. The method of claim 1 in which the library contains more than 10,000,000 samples.

13. The method of claim 1 in which one or more of the chemical penetration enhancers are selected from the group consisting of N-Acyl-hexahydro-2-oxo-1H-azepines, N-Alkyl-dihydro-1,4-oxazepine-5,7-diones, N-Alkylmorpholine-2,3-diones, N-Alkylmorpholine-3,5-diones, Azacycloalkane derivatives (-ketone, -thione), Azacycloalkenone derivatives, 1-[2-(Decylthio)ethyl]azacyclopentan-2-one (HPE-101), N-(2,2), Dihydroxyethyl) dodecylamine, 1-Dodecanoylhexahydro-1-H-azepine, 1-Dodecyl azacycloheptan-2-one (azone or laurocapram), N-Dodecyl diethanolamine, N-Dodecyl-hexahydro-2-thio-1H-azepine, N-Dodecyl-N-(2-methoxyethyl)acetamide, N-Dodecyl-N-(2-methoxyethyl) isobutyramide, N-Dodecyl-piperidine-2-thione, N-Dodecyl-2-piperidinone, N-Dodecyl pyrrolidine-3,5-dione, N-Dodecyl pyrrolidine-2-thione, N-Dodecyl-2-pyrrolidone, 1-Farnesylazacycloheptan-2-one, 1-Farnesylazacyclopentan-2-one, 1-Geranyl azacycloheptan-2-one, 1, Geranylazacyclopentan-2-one, Hexahydro-2-oxo-azepine-1-acetic acid esters, N-(2, Hydroxyethyl)-2-pyrrolidone, 1-Laurylazacycloheptane, 2-(1-Nonyl)-1,3-dioxolane, 1-N-Octylazacyclopentan-2-one, N-(1-Oxododecyl)-hexahydro-1H-azepine, N-(1, Oxododecyl)-morpholines, 1-Oxohydrocarbyl-substituted azacyclohexanes, N-(1-Oxotetradecyl)-hexahydro-2-oxo-1H-azepine, N-(1 Thiododecyl)-morpholines, Acetamide and derivatives, Acetone, n-Alkanes (chain length between 7 and 16), Alkanols, diols, short-chain fatty acids, Cyclohexyl-1,1-dimethylethanol, Dimethyl acetamide, Dimethyl formamide, Ethanol, Ethanol/d-limonene combination, 2-Ethyl-1,3-hexanediol, Ethoxydiglycol (transcutol), Glycerol, Glycols, Lauryl chloride, Limonene, N-Methylformamide, 2-Phenylethanol, 3-Phenyl-1-propanol, 3-Phenyl-2-propen-1-ol, Polyethylene glycol, Polyoxyethylene sorbitan monoesters, Polypropylene glycol 425, Primary alcohols (tridecanol), Procter & Gamble system: small polar solvent (1,2-propane diol, butanediol, C3-6 triols or their mixtures and a polar lipid compound selected form C16 or C18 monounsaturated alcohol, C16 or C18 branched saturated alcohol and their mixtures), Span 20, Squalene, Triacetin, Trichloroethanol, Trifluoroethanol, Trimethylene glycol, Xylene, DMSO, Aliphatic alcohols, Decanol, Lauryl alcohol (dodecanol), Linolenyl alcohol, Nerolidol, 1-Nonanol, n-Octanol, Oleyl alcohol, Butyl acetate, Cetyl lactate, Decyl N,N-dimethylamino acetate, Decyl N,N-dimethylamino isopropionate, Diethyleneglycol oleate, Diethyl sebacate, Diethyl succinate, Diisopropyl sebacate, Dodecyl N,N-dimethylamino acetate Dodecyl (N,N-dimethylamino)-butyrate, Dodecyl N,N-dimethylamino isopropionate, Dodecyl 2-(dimethylamino)propionate, EO-5-oleyl ester, Ethyl acetate, Ethylaceto acetate, Ethyl propionate, Glycerol monoethers, Glycerol monolaurate, Glycerol monooleate, Glycerol monolinoleate, Isopropyl isostearate, Isopropyl linoleate, Isopropyl myristate, Isopropyl myristate/fatty acid monoglyceride combination, Isopropyl myristate/ethanol/L-lactic acid (87:10:3) combination, Isopropyl palmitate, Methyl acetate, Methyl caprate, Methyl laurate, Methyl propionate, Methyl valerate, 1-Monocaproyl glycerol, Monoglycerides (medium chain length), Nicotinic esters (benzyl), Octyl acetate, Octyl N,N-dimethylamino acetate, Oleyl oleate, n-Pentyl N-acetylprolinate, Propylene glycol monolaurate, Sorbitan dilaurate, Sorbitan dioleate, Sorbitan monolaurate, Sorbitan monooleates, Sorbitan trilaurate, Sorbitan trioleate, Sucrose coconut fatty ester mixtures, Sucrose monolaurate, Sucrose monooleate, Tetradecyl N,N-dimethylamino acetate, Alkanoic acids, Capric acid, Diacid, Ethyloctadecanoic acid, Hexanoic acid, Lactic acid, Lauric acid, Linoelaidic acid, Linoleic acid, Linolenic acid, Neodecanoic acid, Oleic acid, Palmitic acid, Pelargonic acid, Propionic acid, Vaccenic acid, a-Monoglyceryl ether, EO-2-oleyl ether, EO-5-oleyl ether, EO-10-oleyl ether, Ether derivatives of polyglycerols and alcohols (1-O-dodecyl-3-O-methyl-2-0-(29,39-dihydroxypropyl)glycerol), L-α-amino-acids, Lecithin, Phospholipids, Saponin/phospholipids, Sodium deoxycholate, Sodium taurocholate, Sodium tauroglycocholate (285), Aliphatic thiols, Alkyl N,N-dialkyl-substituted amino acetates, Anise oil, Anticholinergic agent pretreatment, Ascaridole, Biphasic group derivatives, Bisabolol, Cardamom oil, 1-Carvone, Chenopodium (70% ascaridole), Chenopodium oil, 1,8 Cineole (eucalyptol), Cod liver oil (fatty acid extract), 4-Decyloxazolidin-2-one, Dicyclohexylmethylamine oxide, Diethyl hexadecylphosphonate, Diethyl hexadecylphosphoramidate, N,N-Dimethyl dodecylamine-N-oxide, 4,4-Dimethyl-2-undecyl-2-oxazoline, N-Dodecanoyl-L-amino acid methyl esters, 1,3-Dioxacycloalkanes, (SEPAs), Dithiothreitol, Eucalyptol (cineole), Eucalyptus oil, Eugenol, Herbal extracts, Lactam N-acetic acid esters, N-Hydroxyethalaceamide, 2-Hydroxy-3-oleoyloxy-1-pyroglutamyloxypropane, Menthol, Menthone, Morpholine derivatives, N-Oxide, Nerolidol, Octyl-b-D-(thio)glucopyranosides, Oxazolidinones, piperazine derivatives, Polar lipids, Polydimethylsiloxanes, Poly [2-(methylsulfinyl)ethyl acrylate], Polyrotaxanes, Polyvinylbenzyldimethylalkylammonium chloride, Poly(N-vinyl-N-methyl acetamide), Prodrugs, Saline, Sodium pyroglutaminate, Terpenes and azacyclo ring compounds, Vitamin E (α-tocopherol), Ylang-ylang oil, N-Cyclohexyl-2-pyrrolidone, 1-Butyl-3-dodecyl-2-pyrrolidone, 1,3-Dimethyl-2-imidazolikinone, 1,5 Dimethyl-2-pyrrolidone, 4,4-Dimethyl-2-undecyl-2-oxazoline, 1-Ethyl-2-pyrrolidone, 1-Hexyl-4-methyloxycarbonyl-2-pyrrolidone, 1-Hexyl-2-pyrrolidone, 1-(2 Hydroxyethyl)pyrrolidinone, 3-Hydroxy-N-methyl-2-pyrrolidinone, 1-Isopropyl-2-undecyl-2-imidazoline, 1-Lauryl-4-methyloxycarbonyl-2-pyrrolidone, N-Methyl-2-pyrrolidone, Poly(N-vinylpyrrolidone), Pyroglutamic acid esters, Acid phosphatase, Calonase, Orgelase, Papain, Phospholipase A-2, Phospholipase C, and Triacylglycerol hydrolase.

14. The method of claim 1 wherein the step of measuring the abilities of samples to increase the permeability of a test membrane is accomplished by making a plurality of electrical conductivity measurements and the step of analyzing the measurements is assisted by considering synergy values between chemical penetration enhancers in said samples.

15. The method of claim 1 wherein at least one of the selected compositions contains a pair of chemical penetration enhancers with a synergy value of 2 or more.

16. The method of claim 1 wherein at least one of the selected compositions contains a pair of chemical penetration enhancers with a synergy values of 4 or more.

17. The method of claim 1 wherein the step of analyzing the measurements is assisted by considering one or more potency phase maps.

18. The method of claim 1 wherein the step of analyzing the measurements is assisted by considering synergy values between one or more pairs of chemical penetration enhancers in the samples.

19. The method of claim 1 including the step of determining the irritation potential of the selected compositions whereby to identify one or more compositions having potent ability to increase the permeability of a body surface and low irritation potential.

20. The method of claim 19 in which the determination of irritation potential is accomplished with an in vitro measurement.

21. The method of claim 19 in which the determination of irritation potential is accomplished with an in vivo measurement.

22. The method of claim 19 in which the determination of irritation potential is accomplished using an interleukin-1α assay.

23. The method of claim 19 in which the determination of irritation potential is accomplished using a methyl thiazol tetrazolium assay.

24. The method of claim 19 in which irritation potential is measured using a 21-day cumulative irritation test.

25. The method of claim 19 including the step of combining each identified composition with a selected active component to form one or more candidate active component formulations.

26. The method of claim 25 including the step of testing each candidate active component formulation for the penetration of the active component into or through skin or mucosa.

27. The method of claim 26 in which the candidate active component formulation is placed on porcine or human skin and penetration of the active component through the skin is measured after a suitable incubation time.

28. The method of claim 27 in which penetration of the active component through the skin is measured using a Franz diffusion cell.

29. The method of claim 26 including the step of determining whether the tested candidate active component formulation can deliver the necessary active component amount through the skin.

30. The method of claim 29 in which the capacity of the tested candidate active component formulation to deliver the necessary active component amount through the skin is determined by comparing penetration of the candidate active component formulation with published data.

31. The method of claim 25 wherein the active component is a drug and further including the step of conducting animal testing to confirm the ability of an active component formulation to deliver sufficient drug across the skin to achieve therapeutic levels of the drug in the blood of animals.

32. The method of claim 31 in which the animal testing comprises in vivo experiments on hairless rats performed using leuprolide acetate as a model active component.

33. A composition identified by the method of claim 19 having potent ability to increase the permeability of skin and pharmaceutically acceptable irritation potential.

34. A method for identifying active component formulations comprising a plurality of chemical penetration enhancers having potent ability to increase the permeability of a body surface and low irritation potential, comprising:
(a) providing a library, said library comprising a plurality of samples comprising at least two chemical penetration enhancers;
(b) screening the library with a high throughput device by a method comprising (i) securing mammalian skin or mucosa to a device comprising a donor plate, the donor plate including a plurality of donor holes passing through the donor plate, (ii) introducing said samples into the donor holes, and (iii) measuring the abilities of the samples to increase the permeability of said mammalian skin or mucosa by making a plurality of electrical conductivity measurements;
(c) analyzing said electrical conductivity measurements to select compositions having high synergy values and potent ability to increase the permeability of said body surface;
(d) determining the irritation potential of the selected compositions whereby to identify one or more compositions having potent ability to increase the permeability of a body surface and low irritation potential;
(e) combining the identified compositions with a selected active component to form one or more candidate active component formulations;
(f) testing the candidate active component formulations for penetration of said active component through a body surface;
(g) analyzing the results of the tests of penetration of said active component through said body surface to select an active component formulation having potent ability to increase the permeability of said body surface and low irritation potential.

35. An active component formulation with potent ability to increase the permeability of a body surface and low irritation potential selected according to the method of claim 34.

36. A formulation comprising a first and second chemical penetration enhancer having a 24-hour synergy value of 2 or more.

37. The combination of chemical penetration enhancers of claim 36 in which the synergy value is calculated according to the following equation
$$S = \frac{{ER}_{A + B} ( X , Y )}{X \cdot {ER}_{A} ( Y ) + ( 1 - X ) \cdot {ER}_{B} ( Y )} ,$$
where ER_{A+B}(X,Y) is the 24-hour enhancement ratio obtained with said formulation, A stands for said first chemical penetration enhancer, B stands for said second penetration enhancer, Y stands for the combined total concentration of said first and second chemical penetration enhancer in said formulation measured in weight/volume, X stands for the weight fraction said first chemical penetration enhancer in the formulation divided by Y, and ER_{A}(Y) and ER_{B}(Y) are the 24-hour enhancement ratios obtained with a second and third formulation where the chemical penetration enhancers A and B are replaced in said formulation with pure components A and B, respectively, each at concentration Y weight/volume.

38. The formulation of claim 36 in which the 24-hour synergy value is 4 or more.

39. A formulation comprising a first and second chemical penetration enhancer with potent ability to increase the permeability of skin showing sufficient partitioning of components of said formulation between the stratum corneum of skin and other layers of skin to exhibit low irritation potential.

40. The formulation of claim 39 wherein the 21-day cumulative irritation test score of said formulation is less than about 199.

41. A composition comprising a first and second chemical penetration enhancer having potent ability to increase the permeability of skin and low irritation potential to enable transdermal delivery of a drug having a molecular weight of at least 500 Da with pharmaceutically acceptable irritation potential.

42. A composition comprising sodium laurel ether sulfate and 1-phenyl piperazine having potent ability to increase the permeability of skin and low irritation potential.

43. A composition comprising N-lauryl sarcosine and sorbitan monolaurate having potent ability to increase the permeability of skin and low irritation potential.

44. A formulation for topical and/or transdermal administration of a drug, comprising:
(a) a therapeutically effective amount of said drug;
(b) a pharmaceutically acceptable vehicle suitable for topical or transdermal drug administration;
(c) a first and second chemical penetration enhancer, the synergy value between said first and second chemical penetration enhancer being at least about 2;
wherein said formulation has a pharmaceutically acceptable irritation potential and the skin conductivity enhancement ratio of the formulation is at least about 30.

45. A composition comprising a first and second chemical penetration enhancer wherein the 24-hour synergy value between said first and second chemical penetration enhancer is at least about 2 and wherein the irritation antergy factor between said first and second chemical penetration enhancer is at least about 2, said irritation antergy factor being computed using the MTT 4-hour cell viability percentage measure of irritation potential.

46. The formulation of claim 44 wherein said chemical penetration enhancers are selected from the group consisting of surfactants, azone and related compounds, solvents and related compounds, fatty alcohols, fatty esters and fatty acids.

47. A method for treating a disease that is responsive to administration of a drug comprising applying the formulation of claim 44 to a patient's body surface.

48. A system for topical or transdermal administration of a drug, comprising:
(a) the formulation of claim 44;
(b) at least one drug reservoir, said reservoir containing said formulation;
(c) means for securing said system to a body surface.

49. A transdermal patch comprising the formulation of claim 44.

50. A method for delivering an active component, comprising applying a formulation to the skin of a mammal said formulation comprising:
(a) an effective amount of said active component;
(b) a cosmetically or pharmaceutically acceptable vehicle;
(c) a first chemical penetration enhancer; and
(d) a second chemical penetration enhancer;
wherein said formulation has an irritation potential that is less than that of 1.5% wt/vol oleic acid in a vehicle consisting of phosphate buffered saline, the 24-hour synergy value between the first and second chemical penetration enhancer is at least about 2, and the 24-hour conductivity enhancement ratio of said formulation measured with porcine skin is at least about 30.

51. A method for screening for formulations providing potent ability to increase the permeability of skin and low irritation potential, comprising:
(a) providing a library of samples, a plurality of said samples comprising at least two chemical penetration enhancers;
(b) using a high throughput device to assay the abilities of said samples to permeabilize skin;
(c) analyzing the results of the assay to identify the presence hot spots or suspected hot spots to select one or more compositions for irritation potential measurement; and
(e) measuring the irritation potential of the selected compositions;
whereby formulations providing potent ability to increase the permeability of skin and low irritation potential may be efficiently discovered.

52. A method for making a formulation providing potent ability to deliver an active component and low irritation potential, comprising:
(a) providing at least two materials wherein in aggregate the components of said at least two materials comprise a first and second chemical penetration enhancer, an active component and a vehicle;
(b) combining said at least two materials in a predetermined ratio;
whereby a formulation is made, said formulation having a 24-hour porcine skin conductivity enhancement ratio of at least about 30 and an MTT 4-hour cell viability percentage of less than about 15%.
